SEQUENCE LISTING (723 sequences)

SEQ ID NO: 1 - RNA, 22 nt, Homo Sapiens
ugagguaguagguuguauaguu

SEQ ID NO: 2 - RNA, 21 nt, Homo Sapiens
cuauacaaucuacugucuuuc

SEQ ID NO: 3 - RNA, 22 nt, Homo Sapiens
ugagguaguagguugugugguu

SEQ ID NO: 4 - RNA, 22 nt, Homo Sapiens
cuauacaaccuacugccuuccc

SEQ ID NO: 5 - RNA, 22 nt, Homo Sapiens
ugagguaguagguuguaugguu

SEQ ID NO: 6 - RNA, 22 nt, Homo Sapiens
uagaguuacacccugggaguua

SEQ ID NO: 7 - RNA, 22 nt, Homo Sapiens
agagguaguagguugcauaguu

SEQ ID NO: 8 - RNA, 22 nt, Homo Sapiens
cuauacgaccugcugccuuucu

SEQ ID NO: 9 - RNA, 22 nt, Homo Sapiens
ugagguaggagguuguauaguu

SEQ ID NO: 10 - RNA, 22 nt, Homo Sapiens
cuauacggccuccuagcuuucc

SEQ ID NO: 11 - RNA, 22 nt, Homo Sapiens
ugagguaguagauuguauaguu

SEQ ID NO: 12 - RNA, 22 nt, Homo Sapiens
cuauacaaucuauugccuuccc

SEQ ID NO: 13 - RNA, 22 nt, Homo Sapiens
cuauacagucuacugucuuucc

SEQ ID NO: 14 - RNA, 22 nt, Homo Sapiens
ugagguaguaguuuguacaguu

SEQ ID NO: 15 - RNA, 21 nt, Homo Sapiens
cuguacaggccacugccuugc

SEQ ID NO: 16 - RNA, 22 nt, Homo Sapiens
ugagguaguaguuugugcuguu

SEQ ID NO: 17 - RNA, 22 nt, Homo Sapiens
cugcgcaagcuacugccuugcu

SEQ ID NO: 18 - RNA, 22 nt, Homo Sapiens
uggaauguaaagaaguauguau

SEQ ID NO: 19 - RNA, 22 nt, Homo Sapiens
aacccguagauccgaacuugug

SEQ ID NO: 20 - RNA, 22 nt, Homo Sapiens
caagcuuguaucuauagguaug

SEQ ID NO: 21 - RNA, 21 nt, Homo Sapiens
uacaguacugugauaacugaa

SEQ ID NO: 22 - RNA, 22 nt, Homo Sapiens
caguuaucacagugcugaugcu

SEQ ID NO: 23 - RNA, 23 nt, Homo Sapiens
agcagcauuguacagggcuauga

SEQ ID NO: 24 - RNA, 23 nt, Homo Sapiens
ucaaaugcucagacuccuguggu

SEQ ID NO: 25 - RNA, 22 nt, Homo Sapiens
acggauguuugagcaugugcua

SEQ ID NO: 26 - RNA, 23 nt, Homo Sapiens
aaaagugcuuacagugcagguag

SEQ ID NO: 27 - RNA, 22 nt, Homo Sapiens
cugcaauguaagcacuucuuac

SEQ ID NO: 28 - RNA, 21 nt, Homo Sapiens
uaaagugcugacagugcagau

SEQ ID NO: 29 - RNA, 22 nt, Homo Sapiens
ccgcacuguggguacuugcugc

SEQ ID NO: 30 - RNA, 23 nt, Homo Sapiens
agcagcauuguacagggcuauca

SEQ ID NO: 31 - RNA, 23 nt, Homo Sapiens
uacccuguagauccgaauuugug

SEQ ID NO: 32 - RNA, 22 nt, Homo Sapiens
caaauucguaucuaggggaaua

SEQ ID NO: 33 - RNA, 23 nt, Homo Sapiens
uacccuguagaaccgaauuugug

SEQ ID NO: 34 - RNA, 22 nt, Homo Sapiens
acagauucgauucuaggggaau

SEQ ID NO: 35 - RNA, 22 nt, Homo Sapiens
uggagugugacaaugguguuug

SEQ ID NO: 36 - RNA, 22 nt, Homo Sapiens
aacgccauuaucacacuaaaua

SEQ ID NO: 37 - RNA, 20 nt, Homo Sapiens
uaaggcacgcggugaaugcc

SEQ ID NO: 38 - RNA, 22 nt, Homo Sapiens
cguguucacagcggaccuugau

SEQ ID NO: 39 - RNA, 22 nt, Homo Sapiens
acaggugagguucuugggagcc

SEQ ID NO: 40 - RNA, 24 nt, Homo Sapiens
ucccugagacccuuuaaccuguga

SEQ ID NO: 41 - RNA, 22 nt, Homo Sapiens
ucccugagacccuaacuuguga

SEQ ID NO: 42 - RNA, 22 nt, Homo Sapiens
acggguuaggcucuugggagcu

SEQ ID NO: 43 - RNA, 22 nt, Homo Sapiens
ucacaagucaggcucuugggac

SEQ ID NO: 44 - RNA, 22 nt, Homo Sapiens
ucguaccgugaguaauaaugcg

SEQ ID NO: 45 - RNA, 21 nt, Homo Sapiens
cauuauuacuuuugguacgcg

SEQ ID NO: 46 - RNA, 22 nt, Homo Sapiens
ucggauccgucugagcuuggcu

SEQ ID NO: 47 - RNA, 22 nt, Homo Sapiens
cugaagcucagagggcucugau

SEQ ID NO: 48 - RNA, 21 nt, Homo Sapiens
ucacagugaaccggucucuuu

SEQ ID NO: 49 - RNA, 21 nt, Homo Sapiens
ucacagugaaccggucucuuu

SEQ ID NO: 50 - RNA, 22 nt, Homo Sapiens
aagcccuuaccccaaaaaguau

SEQ ID NO: 51 - RNA, 22 nt, Homo Sapiens
aagcccuuaccccaaaaagcau

SEQ ID NO: 52 - RNA, 21 nt, Homo Sapiens
cuuuuugcggucugggcuugc

SEQ ID NO: 53 - RNA, 22 nt, Homo Sapiens
cagugcaauguuaaaagggcau

SEQ ID NO: 54 - RNA, 22 nt, Homo Sapiens
uucacauugugcuacugucugc

SEQ ID NO: 55 - RNA, 22 nt, Homo Sapiens
cagugcaaugaugaaagggcau

SEQ ID NO: 56 - RNA, 21 nt, Homo Sapiens
acucuuucccuguugcacuac

SEQ ID NO: 57 - RNA, 22 nt, Homo Sapiens
uaacagucuacagccauggucg

SEQ ID NO: 58 - RNA, 22 nt, Homo Sapiens
accguggcuuucgauuguuacu

SEQ ID NO: 59 - RNA, 22 nt, Homo Sapiens
uuugguccccuucaaccagcug

SEQ ID NO: 60 - RNA, 22 nt, Homo Sapiens
uuugguccccuucaaccagcua

SEQ ID NO: 61 - RNA, 22 nt, Homo Sapiens
ugugacugguugaccagagggg

SEQ ID NO: 62 - RNA, 23 nt, Homo Sapiens
uauggcuuuuuauuccuauguga

SEQ ID NO: 63 - RNA, 22 nt, Homo Sapiens
uauagggauuggagccguggcg

SEQ ID NO: 64 - RNA, 23 nt, Homo Sapiens
uauggcuuuucauuccuauguga

SEQ ID NO: 65 - RNA, 22 nt, Homo Sapiens
auguagggcuaaaagccauggg

SEQ ID NO: 66 - RNA, 23 nt, Homo Sapiens
acuccauuuguuuugaugaugga

SEQ ID NO: 67 - RNA, 22 nt, Homo Sapiens
caucaucgucucaaaugagucu

SEQ ID NO: 68 - RNA, 23 nt, Homo Sapiens
uuauugcuuaagaauacgcguag

SEQ ID NO: 69 - RNA, 23 nt, Homo Sapiens
agcugguguugugaaucaggccg

SEQ ID NO: 70 - RNA, 22 nt, Homo Sapiens
gcuacuucacaacaccagggcc

SEQ ID NO: 71 - RNA, 22 nt, Homo Sapiens
gcuauuucacgacaccaggguu

SEQ ID NO: 72 - RNA, 22 nt, Homo Sapiens
ggagacgcggcccuguuggagu

SEQ ID NO: 73 - RNA, 22 nt, Homo Sapiens
ucuacagugcacgugucuccag

SEQ ID NO: 74 - RNA, 21 nt, Homo Sapiens
uaccacaggguagaaccacgg

SEQ ID NO: 75 - RNA, 22 nt, Homo Sapiens
cagugguuuuacccuaugguag

SEQ ID NO: 76 - RNA, 22 nt, Homo Sapiens
uaacacugucugguaaagaugg

SEQ ID NO: 77 - RNA, 22 nt, Homo Sapiens
caucuuccaguacaguguugga

SEQ ID NO: 78 - RNA, 23 nt, Homo Sapiens
uguaguguuuccuacuuuaugga

SEQ ID NO: 79 - RNA, 21 nt, Homo Sapiens
cauaaaguagaaagcacuacu

SEQ ID NO: 80 - RNA, 21 nt, Homo Sapiens
ugagaugaagcacuguagcuc

SEQ ID NO: 81 - RNA, 22 nt, Homo Sapiens
ggugcagugcugcaucucuggu

SEQ ID NO: 82 - RNA, 20 nt, Homo Sapiens
uacaguauagaugauguacu

SEQ ID NO: 83 - RNA, 22 nt, Homo Sapiens
ggauaucaucauauacuguaag

SEQ ID NO: 84 - RNA, 23 nt, Homo Sapiens
guccaguuuucccaggaaucccu

SEQ ID NO: 85 - RNA, 22 nt, Homo Sapiens
ggauuccuggaaauacuguucu

SEQ ID NO: 86 - RNA, 22 nt, Homo Sapiens
ugagaacugaauuccauggguu

SEQ ID NO: 87 - RNA, 22 nt, Homo Sapiens
ccucugaaauucaguucuucag

SEQ ID NO: 88 - RNA, 22 nt, Homo Sapiens
ugcccuguggacucaguucugg

SEQ ID NO: 89 - RNA, 22 nt, Homo Sapiens
ugagaacugaauuccauaggcu

SEQ ID NO: 90 - RNA, 20 nt, Homo Sapiens
guguguggaaaugcuucugc

SEQ ID NO: 91 - RNA, 22 nt, Homo Sapiens
gugugcggaaaugcuucugcua

SEQ ID NO: 92 - RNA, 22 nt, Homo Sapiens
ucagugcacuacagaacuuugu

SEQ ID NO: 93 - RNA, 22 nt, Homo Sapiens
aaaguucugagacacuccgacu

SEQ ID NO: 94 - RNA, 22 nt, Homo Sapiens
ucagugcaucacagaacuuugu

SEQ ID NO: 95 - RNA, 22 nt, Homo Sapiens
aaguucuguuauacacucaggc

SEQ ID NO: 96 - RNA, 23 nt, Homo Sapiens
ucuggcuccgugucuucacuccc

SEQ ID NO: 97 - RNA, 21 nt, Homo Sapiens
agggagggacgggggcugugc

SEQ ID NO: 98 - RNA, 22 nt, Homo Sapiens
ucucccaacccuuguaccagug

SEQ ID NO: 99 - RNA, 22 nt, Homo Sapiens
cugguacaggccugggggacag

SEQ ID NO: 100 - RNA, 21 nt, Homo Sapiens
cuagacugaagcuccuugagg

SEQ ID NO: 101 - RNA, 21 nt, Homo Sapiens
ucgaggagcucacagucuagu

SEQ ID NO: 102 - RNA, 21 nt, Homo Sapiens
ucagugcaugacagaacuugg

SEQ ID NO: 103 - RNA, 22 nt, Homo Sapiens
uugcauagucacaaaagugauc

SEQ ID NO: 104 - RNA, 22 nt, Homo Sapiens
uagguuauccguguugccuucg

SEQ ID NO: 105 - RNA, 22 nt, Homo Sapiens
aaucauacacgguugaccuauu

SEQ ID NO: 106 - RNA, 23 nt, Homo Sapiens
uuaaugcuaaucgugauaggggu

SEQ ID NO: 107 - RNA, 22 nt, Homo Sapiens
cuccuacauauuagcauuaaca

SEQ ID NO: 108 - RNA, 22 nt, Homo Sapiens
uagcagcacauaaugguuugug

SEQ ID NO: 109 - RNA, 22 nt, Homo Sapiens
caggccauauugugcugccuca

SEQ ID NO: 110 - RNA, 22 nt, Homo Sapiens
uagcagcacaucaugguuuaca

SEQ ID NO: 111 - RNA, 22 nt, Homo Sapiens
cgaaucauuauuugcugcucua

SEQ ID NO: 112 - RNA, 22 nt, Homo Sapiens
uagcagcacguaaauauuggcg

SEQ ID NO: 113 - RNA, 22 nt, Homo Sapiens
ccaguauuaacugugcugcuga

SEQ ID NO: 114 - RNA, 22 nt, Homo Sapiens
ccaauauuacugugcugcuuua

SEQ ID NO: 115 - RNA, 23 nt, Homo Sapiens
caaagugcuuacagugcagguag

SEQ ID NO: 116 - RNA, 22 nt, Homo Sapiens
acugcagugaaggcacuuguag

SEQ ID NO: 117 - RNA, 23 nt, Homo Sapiens
aacauucaacgcugucggugagu

SEQ ID NO: 118 - RNA, 22 nt, Homo Sapiens
accaucgaccguugauuguacc

SEQ ID NO: 119 - RNA, 22 nt, Homo Sapiens
accacugaccguugacuguacc

SEQ ID NO: 120 - RNA, 23 nt, Homo Sapiens
aacauucauugcugucggugggu

SEQ ID NO: 121 - RNA, 22 nt, Homo Sapiens
aacauucaaccugucggugagu

SEQ ID NO: 122 - RNA, 22 nt, Homo Sapiens
aaccaucgaccguugaguggac

SEQ ID NO: 123 - RNA, 23 nt, Homo Sapiens
aacauucauuguugucggugggu

SEQ ID NO: 124 - RNA, 24 nt, Homo Sapiens
uuuggcaaugguagaacucacacu

SEQ ID NO: 125 - RNA, 21 nt, Homo Sapiens
ugguucuagacuugccaacua

SEQ ID NO: 126 - RNA, 22 nt, Homo Sapiens
uauggcacugguagaauucacu

SEQ ID NO: 127 - RNA, 22 nt, Homo Sapiens
gugaauuaccgaagggccauaa

SEQ ID NO: 128 - RNA, 22 nt, Homo Sapiens
uggacggagaacugauaagggu

SEQ ID NO: 129 - RNA, 22 nt, Homo Sapiens
uggagagaaaggcaguuccuga

SEQ ID NO: 130 - RNA, 22 nt, Homo Sapiens
aggggcuggcuuuccucugguc

SEQ ID NO: 131 - RNA, 22 nt, Homo Sapiens
caaagaauucuccuuuugggcu

SEQ ID NO: 132 - RNA, 22 nt, Homo Sapiens
gcccaaaggugaauuuuuuggg

SEQ ID NO: 133 - RNA, 22 nt, Homo Sapiens
ucgugucuuguguugcagccgg

SEQ ID NO: 134 - RNA, 22 nt, Homo Sapiens
ggcuacaacacaggacccgggc

SEQ ID NO: 135 - RNA, 21 nt, Homo Sapiens
cucccacaugcaggguuugca

SEQ ID NO: 136 - RNA, 21 nt, Homo Sapiens
caucccuugcaugguggaggg

SEQ ID NO: 137 - RNA, 23 nt, Homo Sapiens
uaaggugcaucuagugcagauag

SEQ ID NO: 138 - RNA, 23 nt, Homo Sapiens
acugcccuaagugcuccuucugg

SEQ ID NO: 139 - RNA, 23 nt, Homo Sapiens
uaaggugcaucuagugcaguuag

SEQ ID NO: 140 - RNA, 22 nt, Homo Sapiens
ugcccuaaaugccccuucuggc

SEQ ID NO: 141 - RNA, 22 nt, Homo Sapiens
ugauauguuugauauauuaggu

SEQ ID NO: 142 - RNA, 21 nt, Homo Sapiens
ugauauguuugauauuggguu

SEQ ID NO: 143 - RNA, 23 nt, Homo Sapiens
caacggaaucccaaaagcagcug

SEQ ID NO: 144 - RNA, 22 nt, Homo Sapiens
gcugcgcuuggauuucgucccc

SEQ ID NO: 145 - RNA, 21 nt, Homo Sapiens
cugaccuaugaauugacagcc

SEQ ID NO: 146 - RNA, 22 nt, Homo Sapiens
cugccaauuccauaggucacag

SEQ ID NO: 147 - RNA, 22 nt, Homo Sapiens
aacuggccuacaaagucccagu

SEQ ID NO: 148 - RNA, 22 nt, Homo Sapiens
ugggucuuugcgggcgagauga

SEQ ID NO: 149 - RNA, 22 nt, Homo Sapiens
aacuggcccucaaagucccgcu

SEQ ID NO: 150 - RNA, 22 nt, Homo Sapiens
cgggguuuugagggcgagauga

SEQ ID NO: 151 - RNA, 22 nt, Homo Sapiens
uguaacagcaacuccaugugga

SEQ ID NO: 152 - RNA, 22 nt, Homo Sapiens
ccaguggggcugcuguuaucug

SEQ ID NO: 153 - RNA, 21 nt, Homo Sapiens
uagcagcacagaaauauuggc

SEQ ID NO: 154 - RNA, 22 nt, Homo Sapiens
ccaauauuggcugugcugcucc

SEQ ID NO: 155 - RNA, 22 nt, Homo Sapiens
uagguaguuucauguuguuggg

SEQ ID NO: 156 - RNA, 22 nt, Homo Sapiens
cggcaacaagaaacugccugag

SEQ ID NO: 157 - RNA, 22 nt, Homo Sapiens
uagguaguuuccuguuguuggg

SEQ ID NO: 158 - RNA, 22 nt, Homo Sapiens
uucaccaccuucuccacccagc

SEQ ID NO: 159 - RNA, 22 nt, Homo Sapiens
gguccagaggggagauagguuc

SEQ ID NO: 160 - RNA, 22 nt, Homo Sapiens
acaguagucugcacauugguua

SEQ ID NO: 161 - RNA, 23 nt, Homo Sapiens
cccaguguucagacuaccuguuc

SEQ ID NO: 162 - RNA, 22 nt, Homo Sapiens
acaguagucugcacauugguua

SEQ ID NO: 163 - RNA, 23 nt, Homo Sapiens
cccaguguuuagacuaucuguuc

SEQ ID NO: 164 - RNA, 23 nt, Homo Sapiens
ugugcaaaucuaugcaaaacuga

SEQ ID NO: 165 - RNA, 22 nt, Homo Sapiens
aguuuugcauaguugcacuaca

SEQ ID NO: 166 - RNA, 23 nt, Homo Sapiens
ugugcaaauccaugcaaaacuga

SEQ ID NO: 167 - RNA, 23 nt, Homo Sapiens
aguuuugcagguuugcauccagc

SEQ ID NO: 168 - RNA, 22 nt, Homo Sapiens
aguuuugcagguuugcauuuca

SEQ ID NO: 169 - RNA, 22 nt, Homo Sapiens
uaacacugucugguaacgaugu

SEQ ID NO: 170 - RNA, 22 nt, Homo Sapiens
caucuuaccggacagugcugga

SEQ ID NO: 171 - RNA, 22 nt, Homo Sapiens
uaauacugccugguaaugauga

SEQ ID NO: 172 - RNA, 22 nt, Homo Sapiens
caucuuacugggcagcauugga

SEQ ID NO: 173 - RNA, 23 nt, Homo Sapiens
uaauacugccggguaaugaugga

SEQ ID NO: 174 - RNA, 22 nt, Homo Sapiens
cgucuuacccagcaguguuugg

SEQ ID NO: 175 - RNA, 20 nt, Homo Sapiens
agagguauagggcaugggaa

SEQ ID NO: 176 - RNA, 22 nt, Homo Sapiens
uuccuaugcauauacuucuuug

SEQ ID NO: 177 - RNA, 22 nt, Homo Sapiens
gugaaauguuuaggaccacuag

SEQ ID NO: 178 - RNA, 22 nt, Homo Sapiens
uucccuuugucauccuaugccu

SEQ ID NO: 179 - RNA, 22 nt, Homo Sapiens
uccuucauuccaccggagucug

SEQ ID NO: 180 - RNA, 22 nt, Homo Sapiens
uggaauguaaggaagugugugg

SEQ ID NO: 181 - RNA, 22 nt, Homo Sapiens
auaagacgagcaaaaagcuugu

SEQ ID NO: 182 - RNA, 22 nt, Homo Sapiens
auaagacgaacaaaagguuugu

SEQ ID NO: 183 - RNA, 23 nt, Homo Sapiens
uaaagugcuuauagugcagguag

SEQ ID NO: 184 - RNA, 22 nt, Homo Sapiens
acugcauuaugagcacuuaaag

SEQ ID NO: 185 - RNA, 23 nt, Homo Sapiens
caaagugcucauagugcagguag

SEQ ID NO: 186 - RNA, 22 nt, Homo Sapiens
acuguaguaugggcacuuccag

SEQ ID NO: 187 - RNA, 22 nt, Homo Sapiens
uagcuuaucagacugauguuga

SEQ ID NO: 188 - RNA, 21 nt, Homo Sapiens
caacaccagucgaugggcugu

SEQ ID NO: 189 - RNA, 22 nt, Homo Sapiens
cugugcgugugacagcggcuga

SEQ ID NO: 190 - RNA, 22 nt, Homo Sapiens
uucccuuugucauccuucgccu

SEQ ID NO: 191 - RNA, 21 nt, Homo Sapiens
uaacagucuccagucacggcc

SEQ ID NO: 192 - RNA, 22 nt, Homo Sapiens
acagcaggcacagacaggcagu

SEQ ID NO: 193 - RNA, 22 nt, Homo Sapiens
ugccugucuacacuugcugugc

SEQ ID NO: 194 - RNA, 21 nt, Homo Sapiens
augaccuaugaauugacagac

SEQ ID NO: 195 - RNA, 22 nt, Homo Sapiens
uaaucucagcuggcaacuguga

SEQ ID NO: 196 - RNA, 22 nt, Homo Sapiens
aaaucucugcaggcaaauguga

SEQ ID NO: 197 - RNA, 23 nt, Homo Sapiens
uacugcaucaggaacugauugga

SEQ ID NO: 198 - RNA, 21 nt, Homo Sapiens
uugugcuugaucuaaccaugu

SEQ ID NO: 199 - RNA, 22 nt, Homo Sapiens
augguuccgucaagcaccaugg

SEQ ID NO: 200 - RNA, 22 nt, Homo Sapiens
caugguucugucaagcaccgcg

SEQ ID NO: 201 - RNA, 22 nt, Homo Sapiens
agaguugagucuggacgucccg

SEQ ID NO: 202 - RNA, 22 nt, Homo Sapiens
agaauuguggcuggacaucugu

SEQ ID NO: 203 - RNA, 21 nt, Homo Sapiens
ugauuguccaaacgcaauucu

SEQ ID NO: 204 - RNA, 22 nt, Homo Sapiens
aagcugccaguugaagaacugu

SEQ ID NO: 205 - RNA, 22 nt, Homo Sapiens
aguucuucaguggcaagcuuua

SEQ ID NO: 206 - RNA, 21 nt, Homo Sapiens
ccacaccguaucugacacuuu

SEQ ID NO: 207 - RNA, 21 nt, Homo Sapiens
ccaccaccgugucugacacuu

SEQ ID NO: 208 - RNA, 22 nt, Homo Sapiens
acacagggcuguugugaagacu

SEQ ID NO: 209 - RNA, 23 nt, Homo Sapiens
agcuacauugucugcuggguuuc

SEQ ID NO: 210 - RNA, 22 nt, Homo Sapiens
accuggcauacaauguagauuu

SEQ ID NO: 211 - RNA, 21 nt, Homo Sapiens
agcuacaucuggcuacugggu

SEQ ID NO: 212 - RNA, 22 nt, Homo Sapiens
cucaguagccaguguagauccu

SEQ ID NO: 213 - RNA, 22 nt, Homo Sapiens
ugucaguuugucaaauacccca

SEQ ID NO: 214 - RNA, 22 nt, Homo Sapiens
cguguauuugacaagcugaguu

SEQ ID NO: 215 - RNA, 21 nt, Homo Sapiens
caagucacuagugguuccguu

SEQ ID NO: 216 - RNA, 21 nt, Homo Sapiens
aucacauugccagggauuucc

SEQ ID NO: 217 - RNA, 22 nt, Homo Sapiens
gggguuccuggggaugggauuu

SEQ ID NO: 218 - RNA, 21 nt, Homo Sapiens
aucacauugccagggauuacc

SEQ ID NO: 219 - RNA, 22 nt, Homo Sapiens
uggguuccuggcaugcugauuu

SEQ ID NO: 220 - RNA, 22 nt, Homo Sapiens
uggcucaguucagcaggaacag

SEQ ID NO: 221 - RNA, 22 nt, Homo Sapiens
ugccuacugagcugauaucagu

SEQ ID NO: 222 - RNA, 22 nt, Homo Sapiens
ugccuacugagcugaaacacag

SEQ ID NO: 223 - RNA, 22 nt, Homo Sapiens
cauugcacuugucucggucuga

SEQ ID NO: 224 - RNA, 21 nt, Homo Sapiens
aggcggagacuugggcaauug

SEQ ID NO: 225 - RNA, 22 nt, Homo Sapiens
uucaaguaauccaggauaggcu

SEQ ID NO: 226 - RNA, 22 nt, Homo Sapiens
ccuauucuugguuacuugcacg

SEQ ID NO: 227 - RNA, 22 nt, Homo Sapiens
ccuauucuugauuacuuguuuc

SEQ ID NO: 228 - RNA, 21 nt, Homo Sapiens
uucaaguaauucaggauaggu

SEQ ID NO: 229 - RNA, 22 nt, Homo Sapiens
ccuguucuccauuacuuggcuc

SEQ ID NO: 230 - RNA, 21 nt, Homo Sapiens
uucacaguggcuaaguuccgc

SEQ ID NO: 231 - RNA, 22 nt, Homo Sapiens
agggcuuagcugcuugugagca

SEQ ID NO: 232 - RNA, 21 nt, Homo Sapiens
uucacaguggcuaaguucugc

SEQ ID NO: 233 - RNA, 22 nt, Homo Sapiens
agagcuuagcugauuggugaac

SEQ ID NO: 234 - RNA, 22 nt, Homo Sapiens
cacuagauugugagcuccugga

SEQ ID NO: 235 - RNA, 22 nt, Homo Sapiens
aaggagcucacagucuauugag

SEQ ID NO: 236 - RNA, 22 nt, Homo Sapiens
gaggguuggguggaggcucucc

SEQ ID NO: 237 - RNA, 21 nt, Homo Sapiens
agggcccccccucaauccugu

SEQ ID NO: 238 - RNA, 21 nt, Homo Sapiens
auguaugugugcaugugcaug

SEQ ID NO: 239 - RNA, 24 nt, Homo Sapiens
agcagaagcagggagguucuccca

SEQ ID NO: 240 - RNA, 22 nt, Homo Sapiens
uaugugggaugguaaaccgcuu

SEQ ID NO: 241 - RNA, 22 nt, Homo Sapiens
ugguuuaccgucccacauacau

SEQ ID NO: 242 - RNA, 22 nt, Homo Sapiens
uagcaccaucugaaaucgguua

SEQ ID NO: 243 - RNA, 22 nt, Homo Sapiens
acugauuucuuuugguguucag

SEQ ID NO: 244 - RNA, 23 nt, Homo Sapiens
uagcaccauuugaaaucaguguu

SEQ ID NO: 245 - RNA, 24 nt, Homo Sapiens
gcugguuucauauggugguuuaga

SEQ ID NO: 246 - RNA, 22 nt, Homo Sapiens
cugguuucacaugguggcuuag

SEQ ID NO: 247 - RNA, 22 nt, Homo Sapiens
uagcaccauuugaaaucgguua

SEQ ID NO: 248 - RNA, 22 nt, Homo Sapiens
ugaccgauuucuccugguguuc

SEQ ID NO: 249 - RNA, 22 nt, Homo Sapiens
uauacaagggcagacucucucu

SEQ ID NO: 250 - RNA, 23 nt, Homo Sapiens
cagugcaauaguauugucaaagc

SEQ ID NO: 251 - RNA, 23 nt, Homo Sapiens
cagugcaaugauauugucaaagc

SEQ ID NO: 252 - RNA, 23 nt, Homo Sapiens
uaagugcuuccauguuuugguga

SEQ ID NO: 253 - RNA, 23 nt, Homo Sapiens
acuuaaacguggauguacuugcu

SEQ ID NO: 254 - RNA, 23 nt, Homo Sapiens
uaagugcuuccauguuuuaguag

SEQ ID NO: 255 - RNA, 22 nt, Homo Sapiens
acuuuaacauggaagugcuuuc

SEQ ID NO: 256 - RNA, 23 nt, Homo Sapiens
uaagugcuuccauguuucagugg

SEQ ID NO: 257 - RNA, 22 nt, Homo Sapiens
uuuaacauggggguaccugcug

SEQ ID NO: 258 - RNA, 23 nt, Homo Sapiens
uaagugcuuccauguuugagugu

SEQ ID NO: 259 - RNA, 22 nt, Homo Sapiens
acuuuaacauggaggcacuugc

SEQ ID NO: 260 - RNA, 22 nt, Homo Sapiens
uguaaacauccucgacuggaag

SEQ ID NO: 261 - RNA, 22 nt, Homo Sapiens
cuuucagucggauguuugcagc

SEQ ID NO: 262 - RNA, 22 nt, Homo Sapiens
uguaaacauccuacacucagcu

SEQ ID NO: 263 - RNA, 22 nt, Homo Sapiens
cugggagguggauguuuacuuc

SEQ ID NO: 264 - RNA, 23 nt, Homo Sapiens
uguaaacauccuacacucucagc

SEQ ID NO: 265 - RNA, 22 nt, Homo Sapiens
cugggagaggguuguuuacucc

SEQ ID NO: 266 - RNA, 22 nt, Homo Sapiens
cugggagaaggcuguuuacucu

SEQ ID NO: 267 - RNA, 22 nt, Homo Sapiens
uguaaacauccccgacuggaag

SEQ ID NO: 268 - RNA, 22 nt, Homo Sapiens
cuuucagucagauguuugcugc

SEQ ID NO: 269 - RNA, 22 nt, Homo Sapiens
uguaaacauccuugacuggaag

SEQ ID NO: 270 - RNA, 22 nt, Homo Sapiens
cuuucagucggauguuuacagc

SEQ ID NO: 271 - RNA, 21 nt, Homo Sapiens
aggcaagaugcuggcauagcu

SEQ ID NO: 272 - RNA, 22 nt, Homo Sapiens
ugcuaugccaacauauugccau

SEQ ID NO: 273 - RNA, 22 nt, Homo Sapiens
uauugcacauuacuaaguugca

SEQ ID NO: 274 - RNA, 22 nt, Homo Sapiens
caauuuagugugugugauauuu

SEQ ID NO: 275 - RNA, 22 nt, Homo Sapiens
aaaagcuggguugagagggcga

SEQ ID NO: 276 - RNA, 21 nt, Homo Sapiens
cacauuacacggucgaccucu

SEQ ID NO: 277 - RNA, 22 nt, Homo Sapiens
aggugguccguggcgcguucgc

SEQ ID NO: 278 - RNA, 20 nt, Homo Sapiens
acugccccaggugcugcugg

SEQ ID NO: 279 - RNA, 23 nt, Homo Sapiens
cgcauccccuagggcauuggugu

SEQ ID NO: 280 - RNA, 23 nt, Homo Sapiens
ccuaguagguguccaguaagugu

SEQ ID NO: 281 - RNA, 20 nt, Homo Sapiens
ccucugggcccuuccuccag

SEQ ID NO: 282 - RNA, 22 nt, Homo Sapiens
cuggcccucucugcccuuccgu

SEQ ID NO: 283 - RNA, 22 nt, Homo Sapiens
aacacaccugguuaaccucuuu

SEQ ID NO: 284 - RNA, 23 nt, Homo Sapiens
gcaaagcacacggccugcagaga

SEQ ID NO: 285 - RNA, 22 nt, Homo Sapiens
ucucugggccugugucuuaggc

SEQ ID NO: 286 - RNA, 21 nt, Homo Sapiens
gccccugggccuauccuagaa

SEQ ID NO: 287 - RNA, 22 nt, Homo Sapiens
cuagguauggucccagggaucc

SEQ ID NO: 288 - RNA, 23 nt, Homo Sapiens
ucaagagcaauaacgaaaaaugu

SEQ ID NO: 289 - RNA, 22 nt, Homo Sapiens
uuuuucauuauugcuccugacc

SEQ ID NO: 290 - RNA, 22 nt, Homo Sapiens
cuccuauaugaugccuuucuuc

SEQ ID NO: 291 - RNA, 21 nt, Homo Sapiens
gaacggcuucauacaggaguu

SEQ ID NO: 292 - RNA, 22 nt, Homo Sapiens
uccagcaucagugauuuuguug

SEQ ID NO: 293 - RNA, 22 nt, Homo Sapiens
aacaauauccuggugcugagug

SEQ ID NO: 294 - RNA, 23 nt, Homo Sapiens
ugagcgccucgacgacagagccg

SEQ ID NO: 295 - RNA, 23 nt, Homo Sapiens
ucccuguccuccaggagcucacg

SEQ ID NO: 296 - RNA, 21 nt, Homo Sapiens
gugcauuguaguugcauugca

SEQ ID NO: 297 - RNA, 22 nt, Homo Sapiens
caauguuuccacagugcaucac

SEQ ID NO: 298 - RNA, 20 nt, Homo Sapiens
gugcauugcuguugcauugc

SEQ ID NO: 299 - RNA, 22 nt, Homo Sapiens
cagugccucggcagugcagccc

SEQ ID NO: 300 - RNA, 22 nt, Homo Sapiens
uuauaaagcaaugagacugauu

SEQ ID NO: 301 - RNA, 22 nt, Homo Sapiens
uccgucucaguuacuuuauagc

SEQ ID NO: 302 - RNA, 23 nt, Homo Sapiens
ucucacacagaaaucgcacccgu

SEQ ID NO: 303 - RNA, 21 nt, Homo Sapiens
aggggugcuaucugugauuga

SEQ ID NO: 304 - RNA, 22 nt, Homo Sapiens
gcugacuccuaguccagggcuc

SEQ ID NO: 305 - RNA, 23 nt, Homo Sapiens
ugucugcccgcaugccugccucu

SEQ ID NO: 306 - RNA, 22 nt, Homo Sapiens
uggcagugucuuagcugguugu

SEQ ID NO: 307 - RNA, 22 nt, Homo Sapiens
caaucagcaaguauacugcccu

SEQ ID NO: 308 - RNA, 22 nt, Homo Sapiens
caaucacuaacuccacugccau

SEQ ID NO: 309 - RNA, 23 nt, Homo Sapiens
uaggcagugucauuagcugauug

SEQ ID NO: 310 - RNA, 22 nt, Homo Sapiens
aaucacuaaccacacggccagg

SEQ ID NO: 311 - RNA, 23 nt, Homo Sapiens
aggcaguguaguuagcugauugc

SEQ ID NO: 312 - RNA, 23 nt, Homo Sapiens
ucccccaggugugauucugauuu

SEQ ID NO: 313 - RNA, 22 nt, Homo Sapiens
uuaucagaaucuccagggguac

SEQ ID NO: 314 - RNA, 22 nt, Homo Sapiens
aacacaccuauucaaggauuca

SEQ ID NO: 315 - RNA, 24 nt, Homo Sapiens
aauccuuggaaccuaggugugagu

SEQ ID NO: 316 - RNA, 22 nt, Homo Sapiens
aauugcacgguauccaucugua

SEQ ID NO: 317 - RNA, 22 nt, Homo Sapiens
cggguggaucacgaugcaauuu

SEQ ID NO: 318 - RNA, 22 nt, Homo Sapiens
uaaugccccuaaaaauccuuau

SEQ ID NO: 319 - RNA, 22 nt, Homo Sapiens
aauugcacuuuagcaaugguga

SEQ ID NO: 320 - RNA, 22 nt, Homo Sapiens
acuguugcuaauaugcaacucu

SEQ ID NO: 321 - RNA, 21 nt, Homo Sapiens
aauaauacaugguugaucuuu

SEQ ID NO: 322 - RNA, 22 nt, Homo Sapiens
agaucgaccguguuauauucgc

SEQ ID NO: 323 - RNA, 22 nt, Homo Sapiens
gccugcugggguggaaccuggu

SEQ ID NO: 324 - RNA, 23 nt, Homo Sapiens
aagugccgccaucuuuugagugu

SEQ ID NO: 325 - RNA, 20 nt, Homo Sapiens
acucaaacugugggggcacu

SEQ ID NO: 326 - RNA, 23 nt, Homo Sapiens
aaagugcugcgacauuugagcgu

SEQ ID NO: 327 - RNA, 23 nt, Homo Sapiens
gaagugcuucgauuuuggggugu

SEQ ID NO: 328 - RNA, 22 nt, Homo Sapiens
acucaaaaugggggcgcuuucc

SEQ ID NO: 329 - RNA, 22 nt, Homo Sapiens
uuauaauacaaccugauaagug

SEQ ID NO: 330 - RNA, 22 nt, Homo Sapiens
cuuaucagauuguauuguaauu

SEQ ID NO: 331 - RNA, 22 nt, Homo Sapiens
auauaauacaaccugcuaagug

SEQ ID NO: 332 - RNA, 22 nt, Homo Sapiens
cuuagcagguuguauuaucauu

SEQ ID NO: 333 - RNA, 22 nt, Homo Sapiens
uuuguucguucggcucgcguga

SEQ ID NO: 334 - RNA, 21 nt, Homo Sapiens
aucauagaggaaaauccacgu

SEQ ID NO: 335 - RNA, 22 nt, Homo Sapiens
guagauucuccuucuaugagua

SEQ ID NO: 336 - RNA, 22 nt, Homo Sapiens
aucauagaggaaaauccauguu

SEQ ID NO: 337 - RNA, 21 nt, Homo Sapiens
aacauagaggaaauuccacgu

SEQ ID NO: 338 - RNA, 22 nt, Homo Sapiens
aucacacaaaggcaacuuuugu

SEQ ID NO: 339 - RNA, 22 nt, Homo Sapiens
agagguugcccuuggugaauuc

SEQ ID NO: 340 - RNA, 21 nt, Homo Sapiens
acuggacuuggagucagaagg

SEQ ID NO: 341 - RNA, 22 nt, Homo Sapiens
cuccugacuccagguccugugu

SEQ ID NO: 342 - RNA, 21 nt, Homo Sapiens
ugguagacuauggaacguagg

SEQ ID NO: 343 - RNA, 22 nt, Homo Sapiens
uauguaacaugguccacuaacu

SEQ ID NO: 344 - RNA, 22 nt, Homo Sapiens
uauguaauaugguccacaucuu

SEQ ID NO: 345 - RNA, 22 nt, Homo Sapiens
ugguugaccauagaacaugcgc

SEQ ID NO: 346 - RNA, 22 nt, Homo Sapiens
uauacaagggcaagcucucugu

SEQ ID NO: 347 - RNA, 22 nt, Homo Sapiens
gaaguuguucgugguggauucg

SEQ ID NO: 348 - RNA, 22 nt, Homo Sapiens
agaucagaaggugauuguggcu

SEQ ID NO: 349 - RNA, 20 nt, Homo Sapiens
auuccuagaaauuguucaua

SEQ ID NO: 350 - RNA, 22 nt, Homo Sapiens
gaauguugcucggugaaccccu

SEQ ID NO: 351 - RNA, 23 nt, Homo Sapiens
agguuacccgagcaacuuugcau

SEQ ID NO: 352 - RNA, 21 nt, Homo Sapiens
aauauaacacagauggccugu

SEQ ID NO: 353 - RNA, 21 nt, Homo Sapiens
uaguagaccguauagcguacg

SEQ ID NO: 354 - RNA, 22 nt, Homo Sapiens
uauguaacacgguccacuaacc

SEQ ID NO: 355 - RNA, 23 nt, Homo Sapiens
acuucaccugguccacuagccgu

SEQ ID NO: 356 - RNA, 23 nt, Homo Sapiens
aucaacagacauuaauugggcgc

SEQ ID NO: 357 - RNA, 22 nt, Homo Sapiens
acuggacuuagggucagaaggc

SEQ ID NO: 358 - RNA, 23 nt, Homo Sapiens
agcucggucugaggccccucagu

SEQ ID NO: 359 - RNA, 23 nt, Homo Sapiens
ugaggggcagagagcgagacuuu

SEQ ID NO: 360 - RNA, 22 nt, Homo Sapiens
cagcagcaauucauguuuugaa

SEQ ID NO: 361 - RNA, 21 nt, Homo Sapiens
caaaacgugaggcgcugcuau

SEQ ID NO: 362 - RNA, 23 nt, Homo Sapiens
aaugacacgaucacucccguuga

SEQ ID NO: 363 - RNA, 22 nt, Homo Sapiens
aucgggaaugucguguccgccc

SEQ ID NO: 364 - RNA, 22 nt, Homo Sapiens
uaauacugucugguaaaaccgu

SEQ ID NO: 365 - RNA, 21 nt, Homo Sapiens
ugucuugcaggccgucaugca

SEQ ID NO: 366 - RNA, 22 nt, Homo Sapiens
caggucgucuugcagggcuucu

SEQ ID NO: 367 - RNA, 23 nt, Homo Sapiens
ucuuggaguaggucauugggugg

SEQ ID NO: 368 - RNA, 21 nt, Homo Sapiens
cuggauggcuccuccaugucu

SEQ ID NO: 369 - RNA, 22 nt, Homo Sapiens
aucaugaugggcuccucggugu

SEQ ID NO: 370 - RNA, 22 nt, Homo Sapiens
uugcauauguaggaugucccau

SEQ ID NO: 371 - RNA, 22 nt, Homo Sapiens
uggcaguguauuguuagcuggu

SEQ ID NO: 372 - RNA, 22 nt, Homo Sapiens
aggcaguguauuguuagcuggc

SEQ ID NO: 373 - RNA, 22 nt, Homo Sapiens
uuuugcgauguguuccuaauau

SEQ ID NO: 374 - RNA, 22 nt, Homo Sapiens
uugggaucauuuugcauccaua

SEQ ID NO: 375 - RNA, 22 nt, Homo Sapiens
uuuugcaauauguuccugaaua

SEQ ID NO: 376 - RNA, 22 nt, Homo Sapiens
aaaccguuaccauuacugaguu

SEQ ID NO: 377 - RNA, 22 nt, Homo Sapiens
aacuguuugcagaggaaacuga

SEQ ID NO: 378 - RNA, 22 nt, Homo Sapiens
cucaucugcaaagaaguaagug

SEQ ID NO: 379 - RNA, 23 nt, Homo Sapiens
agguuguccguggugaguucgca

SEQ ID NO: 380 - RNA, 23 nt, Homo Sapiens
uagugcaauauugcuuauagggu

SEQ ID NO: 381 - RNA, 22 nt, Homo Sapiens
acccuaucaauauugucucugc

SEQ ID NO: 382 - RNA, 21 nt, Homo Sapiens
gcaguccaugggcauauacac

SEQ ID NO: 383 - RNA, 22 nt, Homo Sapiens
uaugugccuuuggacuacaucg

SEQ ID NO: 384 - RNA, 21 nt, Homo Sapiens
ucacuccucuccucccgucuu

SEQ ID NO: 385 - RNA, 22 nt, Homo Sapiens
aagacgggaggaaagaagggag

SEQ ID NO: 386 - RNA, 22 nt, Homo Sapiens
ucaggcucaguccccucccgau

SEQ ID NO: 387 - RNA, 22 nt, Homo Sapiens
gucauacacggcucuccucucu

SEQ ID NO: 388 - RNA, 22 nt, Homo Sapiens
agaggcuggccgugaugaauuc

SEQ ID NO: 389 - RNA, 21 nt, Homo Sapiens
cggggcagcucaguacaggau

SEQ ID NO: 390 - RNA, 22 nt, Homo Sapiens
uccuguacugagcugccccgag

SEQ ID NO: 391 - RNA, 22 nt, Homo Sapiens
aaucauacagggacauccaguu

SEQ ID NO: 392 - RNA, 22 nt, Homo Sapiens
aaucguacagggucauccacuu

SEQ ID NO: 393 - RNA, 21 nt, Homo Sapiens
uugaaaggcuauuucuugguc

SEQ ID NO: 394 - RNA, 21 nt, Homo Sapiens
cccagauaauggcacucucaa

SEQ ID NO: 395 - RNA, 22 nt, Homo Sapiens
gugacaucacauauacggcagc

SEQ ID NO: 396 - RNA, 22 nt, Homo Sapiens
caaccuggaggacuccaugcug

SEQ ID NO: 397 - RNA, 20 nt, Homo Sapiens
ccauggaucuccaggugggu

SEQ ID NO: 398 - RNA, 22 nt, Homo Sapiens
cuuaugcaagauucccuucuac

SEQ ID NO: 399 - RNA, 22 nt, Homo Sapiens
aguggggaacccuuccaugagg

SEQ ID NO: 400 - RNA, 23 nt, Homo Sapiens
aggaccugcgggacaagauucuu

SEQ ID NO: 401 - RNA, 22 nt, Homo Sapiens
ugaaggucuacugugugccagg

SEQ ID NO: 402 - RNA, 22 nt, Homo Sapiens
uuguacaugguaggcuuucauu

SEQ ID NO: 403 - RNA, 22 nt, Homo Sapiens
ugaaacauacacgggaaaccuc

SEQ ID NO: 404 - RNA, 22 nt, Homo Sapiens
aaacaaacauggugcacuucuu

SEQ ID NO: 405 - RNA, 22 nt, Homo Sapiens
ugaguauuacauggccaaucuc

SEQ ID NO: 406 - RNA, 21 nt, Homo Sapiens
cagcagcacacugugguuugu

SEQ ID NO: 407 - RNA, 22 nt, Homo Sapiens
caaaccacacugugguguuaga

SEQ ID NO: 408 - RNA, 23 nt, Homo Sapiens
uuucaagccagggggcguuuuuc

SEQ ID NO: 409 - RNA, 22 nt, Homo Sapiens
aacaucacagcaagucugugcu

SEQ ID NO: 410 - RNA, 21 nt, Homo Sapiens
uuaagacuugcagugauguuu

SEQ ID NO: 411 - RNA, 23 nt, Homo Sapiens
uaauccuugcuaccugggugaga

SEQ ID NO: 412 - RNA, 22 nt, Homo Sapiens
augcaccugggcaaggauucug

SEQ ID NO: 413 - RNA, 22 nt, Homo Sapiens
aaugcacccgggcaaggauucu

SEQ ID NO: 414 - RNA, 22 nt, Homo Sapiens
aauccuuugucccugggugaga

SEQ ID NO: 415 - RNA, 22 nt, Homo Sapiens
aaugcaccugggcaaggauuca

SEQ ID NO: 416 - RNA, 21 nt, Homo Sapiens
auccuugcuaucugggugcua

SEQ ID NO: 417 - RNA, 23 nt, Homo Sapiens
uagcagcgggaacaguucugcag

SEQ ID NO: 418 - RNA, 22 nt, Homo Sapiens
agacccuggucugcacucuauc

SEQ ID NO: 419 - RNA, 22 nt, Homo Sapiens
cgucaacacuugcugguuuccu

SEQ ID NO: 420 - RNA, 22 nt, Homo Sapiens
gggagccaggaaguauugaugu

SEQ ID NO: 421 - RNA, 21 nt, Homo Sapiens
uaaggcacccuucugaguaga

SEQ ID NO: 422 - RNA, 21 nt, Homo Sapiens
uuuugcaccuuuuggagugaa

SEQ ID NO: 423 - RNA, 23 nt, Homo Sapiens
ugauuguagccuuuuggaguaga

SEQ ID NO: 424 - RNA, 23 nt, Homo Sapiens
uacuccagagggcgucacucaug

SEQ ID NO: 425 - RNA, 22 nt, Homo Sapiens
uacugcagacguggcaaucaug

SEQ ID NO: 426 - RNA, 22 nt, Homo Sapiens
ugauugguacgucuguggguag

SEQ ID NO: 427 - RNA, 21 nt, Homo Sapiens
uacugcagacaguggcaauca

SEQ ID NO: 428 - RNA, 22 nt, Homo Sapiens
uacucaggagaguggcaaucac

SEQ ID NO: 429 - RNA, 21 nt, Homo Sapiens
gugucuuuugcucugcaguca

SEQ ID NO: 430 - RNA, 22 nt, Homo Sapiens
aagugcugucauagcugagguc

SEQ ID NO: 431 - RNA, 23 nt, Homo Sapiens
cacucagccuugagggcacuuuc

SEQ ID NO: 432 - RNA, 23 nt, Homo Sapiens
uaaauuucaccuuucugagaagg

SEQ ID NO: 433 - RNA, 18 nt, Homo Sapiens
uucacagggaggugucau

SEQ ID NO: 434 - RNA, 21 nt, Homo Sapiens
auugacacuucugugaguaga

SEQ ID NO: 435 - RNA, 22 nt, Homo Sapiens
gagugccuucuuuuggagcguu

SEQ ID NO: 436 - RNA, 24 nt, Homo Sapiens
uucuccaaaagaaagcacuuucug

SEQ ID NO: 437 - RNA, 18 nt, Homo Sapiens
ugcuuccuuucagagggu

SEQ ID NO: 438 - RNA, 23 nt, Homo Sapiens
uucucgaggaaagaagcacuuuc

SEQ ID NO: 439 - RNA, 22 nt, Homo Sapiens
aucuggagguaagaagcacuuu

SEQ ID NO: 440 - RNA, 18 nt, Homo Sapiens
ugcuuccuuucagagggu

SEQ ID NO: 441 - RNA, 22 nt, Homo Sapiens
ccucuagauggaagcacugucu

SEQ ID NO: 442 - RNA, 22 nt, Homo Sapiens
aucgugcaucccuuuagagugu

SEQ ID NO: 443 - RNA, 22 nt, Homo Sapiens
ucgugcaucccuuuagaguguu

SEQ ID NO: 444 - RNA, 22 nt, Homo Sapiens
aucgugcauccuuuuagagugu

SEQ ID NO: 445 - RNA, 22 nt, Homo Sapiens
gaaagcgcuucccuuugcugga

SEQ ID NO: 446 - RNA, 20 nt, Homo Sapiens
cugcaaagggaagcccuuuc

SEQ ID NO: 447 - RNA, 22 nt, Homo Sapiens
caaagcgcuccccuuuagaggu

SEQ ID NO: 448 - RNA, 23 nt, Homo Sapiens
caaagcgcuucucuuuagagugu

SEQ ID NO: 449 - RNA, 23 nt, Homo Sapiens
ucucuggagggaagcacuuucug

SEQ ID NO: 450 - RNA, 21 nt, Homo Sapiens
caaagcgcuucccuuuggagc

SEQ ID NO: 451 - RNA, 22 nt, Homo Sapiens
cucuagagggaagcacuuucug

SEQ ID NO: 452 - RNA, 21 nt, Homo Sapiens
aaagcgcuucccuucagagug

SEQ ID NO: 453 - RNA, 22 nt, Homo Sapiens
cucuagagggaagcgcuuucug

SEQ ID NO: 454 - RNA, 21 nt, Homo Sapiens
gaaagcgcuucucuuuagagg

SEQ ID NO: 455 - RNA, 22 nt, Homo Sapiens
cucuagagggaagcacuuucuc

SEQ ID NO: 456 - RNA, 22 nt, Homo Sapiens
aaagugcauccuuuuagagugu

SEQ ID NO: 457 - RNA, 22 nt, Homo Sapiens
cucuagagggaagcgcuuucug

SEQ ID NO: 458 - RNA, 22 nt, Homo Sapiens
aaagugcauccuuuuagagguu

SEQ ID NO: 459 - RNA, 22 nt, Homo Sapiens
cucuagagggaagcgcuuucug

SEQ ID NO: 460 - RNA, 22 nt, Homo Sapiens
aaagugcaucuuuuuagaggau

SEQ ID NO: 461 - RNA, 22 nt, Homo Sapiens
cucuagagggaagcgcuuucug

SEQ ID NO: 462 - RNA, 22 nt, Homo Sapiens
caaagugccucccuuuagagug

SEQ ID NO: 463 - RNA, 22 nt, Homo Sapiens
aagugccuccuuuuagaguguu

SEQ ID NO: 464 - RNA, 22 nt, Homo Sapiens
uucuccaaaagggagcacuuuc

SEQ ID NO: 465 - RNA, 22 nt, Homo Sapiens
aaagugcuucccuuuggacugu

SEQ ID NO: 466 - RNA, 21 nt, Homo Sapiens
cuccagagggaaguacuuucu

SEQ ID NO: 467 - RNA, 21 nt, Homo Sapiens
aaagugcuuccuuuuagaggg

SEQ ID NO: 468 - RNA, 22 nt, Homo Sapiens
aaagugcuuccuuuuagagggu

SEQ ID NO: 469 - RNA, 22 nt, Homo Sapiens
cucuagagggaagcacuuucug

SEQ ID NO: 470 - RNA, 22 nt, Homo Sapiens
aaagugcuucucuuuggugggu

SEQ ID NO: 471 - RNA, 20 nt, Homo Sapiens
cuacaaagggaagcccuuuc

SEQ ID NO: 472 - RNA, 21 nt, Homo Sapiens
aaagugcuuccuuuuugaggg

SEQ ID NO: 473 - RNA, 22 nt, Homo Sapiens
aagugcuuccuuuuagaggguu

SEQ ID NO: 474 - RNA, 24 nt, Homo Sapiens
acaaagugcuucccuuuagagugu

SEQ ID NO: 475 - RNA, 22 nt, Homo Sapiens
acaaagugcuucccuuuagagu

SEQ ID NO: 476 - RNA, 22 nt, Homo Sapiens
aacgcacuucccuuuagagugu

SEQ ID NO: 477 - RNA, 22 nt, Homo Sapiens
aaaaugguucccuuuagagugu

SEQ ID NO: 478 - RNA, 22 nt, Homo Sapiens
cucuagagggaagcgcuuucug

SEQ ID NO: 479 - RNA, 23 nt, Homo Sapiens
gaacgcgcuucccuauagagggu

SEQ ID NO: 480 - RNA, 22 nt, Homo Sapiens
cucuagagggaagcgcuuucug

SEQ ID NO: 481 - RNA, 21 nt, Homo Sapiens
gaaggcgcuucccuuuggagu

SEQ ID NO: 482 - RNA, 22 nt, Homo Sapiens
cuacaaagggaagcacuuucuc

SEQ ID NO: 483 - RNA, 22 nt, Homo Sapiens
gaaggcgcuucccuuuagagcg

SEQ ID NO: 484 - RNA, 21 nt, Homo Sapiens
cuccagagggaugcacuuucu

SEQ ID NO: 485 - RNA, 22 nt, Homo Sapiens
cucuagagggaagcacuuucug

SEQ ID NO: 486 - RNA, 23 nt, Homo Sapiens
cucuugagggaagcacuuucugu

SEQ ID NO: 487 - RNA, 22 nt, Homo Sapiens
gaaagugcuuccuuuuagaggc

SEQ ID NO: 488 - RNA, 20 nt, Homo Sapiens
cugcaaagggaagcccuuuc

SEQ ID NO: 489 - RNA, 22 nt, Homo Sapiens
ccucccacacccaaggcuugca

SEQ ID NO: 490 - RNA, 22 nt, Homo Sapiens
caugccuugaguguaggaccgu

SEQ ID NO: 491 - RNA, 22 nt, Homo Sapiens
ggagaaauuauccuuggugugu

SEQ ID NO: 492 - RNA, 22 nt, Homo Sapiens
uggugggcacagaaucuggacu

SEQ ID NO: 493 - RNA, 25 nt, Homo Sapiens
aaaggauucugcugucggucccacu

SEQ ID NO: 494 - RNA, 22 nt, Homo Sapiens
ugugacagauugauaacugaaa

SEQ ID NO: 495 - RNA, 23 nt, Homo Sapiens
ucggggaucaucaugucacgaga

SEQ ID NO: 496 - RNA, 22 nt, Homo Sapiens
aaacauucgcggugcacuucuu

SEQ ID NO: 497 - RNA, 22 nt, Homo Sapiens
auucugcauuuuuagcaaguuc

SEQ ID NO: 498 - RNA, 22 nt, Homo Sapiens
ucagcaaacauuuauugugugc

SEQ ID NO: 499 - RNA, 22 nt, Homo Sapiens
ucaguaaauguuuauuagauga

SEQ ID NO: 500 - RNA, 22 nt, Homo Sapiens
caaaacuggcaauuacuuuugc

SEQ ID NO: 501 - RNA, 22 nt, Homo Sapiens
aaaaguaauugcgaguuuuacc

SEQ ID NO: 502 - RNA, 22 nt, Homo Sapiens
caagaaccucaguugcuuuugu

SEQ ID NO: 503 - RNA, 22 nt, Homo Sapiens
aaaaguaauugugguuuuggcc

SEQ ID NO: 504 - RNA, 22 nt, Homo Sapiens
caaaaaucucaauuacuuuugc

SEQ ID NO: 505 - RNA, 22 nt, Homo Sapiens
aaaaguaauugcgguuuuugcc

SEQ ID NO: 506 - RNA, 22 nt, Homo Sapiens
caaaaaccacaguuucuuuugc

SEQ ID NO: 507 - RNA, 22 nt, Homo Sapiens
aaaaguaauugugguuuuugcc

SEQ ID NO: 508 - RNA, 21 nt, Homo Sapiens
ugacaacuauggaugagcucu

SEQ ID NO: 509 - RNA, 23 nt, Homo Sapiens
agugccugagggaguaagagccc

SEQ ID NO: 510 - RNA, 22 nt, Homo Sapiens
ugucuuacucccucaggcacau

SEQ ID NO: 511 - RNA, 21 nt, Homo Sapiens
gcgacccacucuugguuucca

SEQ ID NO: 512 - RNA, 21 nt, Homo Sapiens
gcgacccauacuugguuucag

SEQ ID NO: 513 - RNA, 22 nt, Homo Sapiens
gaaaucaagcgugggugagacc

SEQ ID NO: 514 - RNA, 21 nt, Homo Sapiens
aacaggugacugguuagacaa

SEQ ID NO: 515 - RNA, 21 nt, Homo Sapiens
aaaacggugagauuuuguuuu

SEQ ID NO: 516 - RNA, 21 nt, Homo Sapiens
gcuaguccugacucagccagu

SEQ ID NO: 517 - RNA, 21 nt, Homo Sapiens
aggguaagcugaaccucugau

SEQ ID NO: 518 - RNA, 22 nt, Homo Sapiens
auauuaccauuagcucaucuuu

SEQ ID NO: 519 - RNA, 22 nt, Homo Sapiens
gaugagcucauuguaauaugag

SEQ ID NO: 520 - RNA, 23 nt, Homo Sapiens
guuugcacgggugggccuugucu

SEQ ID NO: 521 - RNA, 19 nt, Homo Sapiens
ugagcugcuguaccaaaau

SEQ ID NO: 522 - RNA, 21 nt, Homo Sapiens
uaaaguaaauaugcaccaaaa

SEQ ID NO: 523 - RNA, 20 nt, Homo Sapiens
gcgugcgccggccggccgcc

SEQ ID NO: 524 - RNA, 22 nt, Homo Sapiens
caaaguuuaagauccuugaagu

SEQ ID NO: 525 - RNA, 20 nt, Homo Sapiens
aaaguagcuguaccauuugc

SEQ ID NO: 526 - RNA, 19 nt, Homo Sapiens
agguugacauacguuuccc

SEQ ID NO: 527 - RNA, 19 nt, Homo Sapiens
aggcacggugucagcaggc

SEQ ID NO: 528 - RNA, 22 nt, Homo Sapiens
ggcuggcucgcgaugucuguuu

SEQ ID NO: 529 - RNA, 19 nt, Homo Sapiens
gggcgccugugaucccaac

SEQ ID NO: 530 - RNA, 23 nt, Homo Sapiens
aguauguucuuccaggacagaac

SEQ ID NO: 531 - RNA, 20 nt, Homo Sapiens
auguauaaauguauacacac

SEQ ID NO: 532 - RNA, 21 nt, Homo Sapiens
aguuaaugaauccuggaaagu

SEQ ID NO: 533 - RNA, 22 nt, Homo Sapiens
cgaaaacagcaauuaccuuugc

SEQ ID NO: 534 - RNA, 21 nt, Homo Sapiens
ugaguuggccaucugagugag

SEQ ID NO: 535 - RNA, 20 nt, Homo Sapiens
guccgcucggcgguggccca

SEQ ID NO: 536 - RNA, 24 nt, Homo Sapiens
cugaagugauguguaacugaucag

SEQ ID NO: 537 - RNA, 22 nt, Homo Sapiens
cacgcucaugcacacacccaca

SEQ ID NO: 538 - RNA, 23 nt, Homo Sapiens
ugagugugugugugugagugugu

SEQ ID NO: 539 - RNA, 19 nt, Homo Sapiens
gagccaguuggacaggagc

SEQ ID NO: 540 - RNA, 22 nt, Homo Sapiens
aagauguggaaaaauuggaauc

SEQ ID NO: 541 - RNA, 22 nt, Homo Sapiens
auucuaauuucuccacgucuuu

SEQ ID NO: 542 - RNA, 21 nt, Homo Sapiens
uagauaaaauauugguaccug

SEQ ID NO: 543 - RNA, 21 nt, Homo Sapiens
cuucuugugcucuaggauugu

SEQ ID NO: 544 - RNA, 23 nt, Homo Sapiens
uucauuugguauaaaccgcgauu

SEQ ID NO: 545 - RNA, 22 nt, Homo Sapiens
uugagaaugaugaaucauuagg

SEQ ID NO: 546 - RNA, 21 nt, Homo Sapiens
ucuuguguucucuagaucagu

SEQ ID NO: 547 - RNA, 22 nt, Homo Sapiens
uaacugguugaacaacugaacc

SEQ ID NO: 548 - RNA, 23 nt, Homo Sapiens
uuacaguuguucaaccaguuacu

SEQ ID NO: 549 - RNA, 21 nt, Homo Sapiens
caaagaggaaggucccauuac

SEQ ID NO: 550 - RNA, 22 nt, Homo Sapiens
uuaugguuugccugggacugag

SEQ ID NO: 551 - RNA, 19 nt, Homo Sapiens
ugggcguaucuguaugcua

SEQ ID NO: 552 - RNA, 22 nt, Homo Sapiens
uaugcauuguauuuuuaggucc

SEQ ID NO: 553 - RNA, 21 nt, Homo Sapiens
uuuccauaggugaugagucac

SEQ ID NO: 554 - RNA, 21 nt, Homo Sapiens
uuggccacaauggguuagaac

SEQ ID NO: 555 - RNA, 22 nt, Homo Sapiens
ugagaaccacgucugcucugag

SEQ ID NO: 556 - RNA, 24 nt, Homo Sapiens
ucagaacaaaugccgguucccaga

SEQ ID NO: 557 - RNA, 21 nt, Homo Sapiens
uaauuuuauguauaagcuagu

SEQ ID NO: 558 - RNA, 22 nt, Homo Sapiens
gagcuuauucauaaaagugcag

SEQ ID NO: 559 - RNA, 20 nt, Homo Sapiens
agaccauggguucucauugu

SEQ ID NO: 560 - RNA, 22 nt, Homo Sapiens
uugugucaauaugcgaugaugu

SEQ ID NO: 561 - RNA, 19 nt, Homo Sapiens
ugucucugcugggguuucu

SEQ ID NO: 562 - RNA, 25 nt, Homo Sapiens
aggcaccagccaggcauugcucagc

SEQ ID NO: 563 - RNA, 21 nt, Homo Sapiens
gaagugugccguggugugucu

SEQ ID NO: 564 - RNA, 21 nt, Homo Sapiens
aagccugcccggcuccucggg

SEQ ID NO: 565 - RNA, 22 nt, Homo Sapiens
ugugucacucgaugaccacugu

SEQ ID NO: 566 - RNA, 22 nt, Homo Sapiens
uacgucaucguugucaucguca

SEQ ID NO: 567 - RNA, 20 nt, Homo Sapiens
guugugucaguuuaucaaac

SEQ ID NO: 568 - RNA, 23 nt, Homo Sapiens
acuuacagacaagagccuugcuc

SEQ ID NO: 569 - RNA, 22 nt, Homo Sapiens
uggucuaggauuguuggaggag

SEQ ID NO: 570 - RNA, 23 nt, Homo Sapiens
gacacgggcgacagcugcggccc

SEQ ID NO: 571 - RNA, 22 nt, Homo Sapiens
cacacacugcaauuacuuuugc

SEQ ID NO: 572 - RNA, 19 nt, Homo Sapiens
aggcugcggaauucaggac

SEQ ID NO: 573 - RNA, 23 nt, Homo Sapiens
uaaaucccauggugccuucuccu

SEQ ID NO: 574 - RNA, 21 nt, Homo Sapiens
aaacuacugaaaaucaaagau

SEQ ID NO: 575 - RNA, 21 nt, Homo Sapiens
guucaaauccagaucuauaac

SEQ ID NO: 576 - RNA, 25 nt, Homo Sapiens
aggggugguguugggacagcuccgu

SEQ ID NO: 577 - RNA, 20 nt, Homo Sapiens
aggguguuucucucaucucu

SEQ ID NO: 578 - RNA, 21 nt, Homo Sapiens
ugagcuaaaugugugcuggga

SEQ ID NO: 579 - RNA, 23 nt, Homo Sapiens
gcgaggaccccucggggucugac

SEQ ID NO: 580 - RNA, 25 nt, Homo Sapiens
gcugggcagggcuucugagcuccuu

SEQ ID NO: 581 - RNA, 20 nt, Homo Sapiens
aggaauguuccuucuuugcc

SEQ ID NO: 582 - RNA, 23 nt, Homo Sapiens
gaacgccuguucuugccaggugg

SEQ ID NO: 583 - RNA, 22 nt, Homo Sapiens
uccgagccugggucucccucuu

SEQ ID NO: 584 - RNA, 22 nt, Homo Sapiens
ggggguccccggugcucggauc

SEQ ID NO: 585 - RNA, 22 nt, Homo Sapiens
agucauuggaggguuugagcag

SEQ ID NO: 586 - RNA, 22 nt, Homo Sapiens
acucaaaacccuucagugacuu

SEQ ID NO: 587 - RNA, 22 nt, Homo Sapiens
agacuucccauuugaagguggc

SEQ ID NO: 588 - RNA, 23 nt, Homo Sapiens
aaacucuacuuguccuucugagu

SEQ ID NO: 589 - RNA, 24 nt, Homo Sapiens
gaccuggacauguuugugcccagu

SEQ ID NO: 590 - RNA, 20 nt, Homo Sapiens
auggagauagauauagaaau

SEQ ID NO: 591 - RNA, 21 nt, Homo Sapiens
ggcuagcaacagcgcuuaccu

SEQ ID NO: 592 - RNA, 21 nt, Homo Sapiens
acagucugcugagguuggagc

SEQ ID NO: 593 - RNA, 23 nt, Homo Sapiens
aucccuugcaggggcuguugggu

SEQ ID NO: 594 - RNA, 21 nt, Homo Sapiens
cacaagguauugguauuaccu

SEQ ID NO: 595 - RNA, 22 nt, Homo Sapiens
uaguaccaguaccuuguguuca

SEQ ID NO: 596 - RNA, 21 nt, Homo Sapiens
agggggaaaguucuauagucc

SEQ ID NO: 597 - RNA, 22 nt, Homo Sapiens
gacuauagaacuuucccccuca

SEQ ID NO: 598 - RNA, 19 nt, Homo Sapiens
agcugucugaaaaugucuu

SEQ ID NO: 599 - RNA, 22 nt, Homo Sapiens
gugagucucuaagaaaagagga

SEQ ID NO: 600 - RNA, 21 nt, Homo Sapiens
ucuaguaagaguggcagucga

SEQ ID NO: 601 - RNA, 22 nt, Homo Sapiens
augcugacauauuuacuagagg

SEQ ID NO: 602 - RNA, 21 nt, Homo Sapiens
uggguuuacguugggagaacu

SEQ ID NO: 603 - RNA, 22 nt, Homo Sapiens
guucucccaacguaagcccagc

SEQ ID NO: 604 - RNA, 22 nt, Homo Sapiens
aguauucuguaccagggaaggu

SEQ ID NO: 605 - RNA, 21 nt, Homo Sapiens
agaccuggcccagaccucagc

SEQ ID NO: 606 - RNA, 19 nt, Homo Sapiens
gugucugcuuccuguggga

SEQ ID NO: 607 - RNA, 23 nt, Homo Sapiens
cuaauaguaucuaccacaauaaa

SEQ ID NO: 608 - RNA, 22 nt, Homo Sapiens
aaccagcaccccaacuuuggac

SEQ ID NO: 609 - RNA, 23 nt, Homo Sapiens
acuugggcacugaaacaaugucc

SEQ ID NO: 610 - RNA, 23 nt, Homo Sapiens
ugugcuugcucgucccgcccgca

SEQ ID NO: 611 - RNA, 24 nt, Homo Sapiens
acugggggcuuucgggcucugcgu

SEQ ID NO: 612 - RNA, 25 nt, Homo Sapiens
agggaucgcgggcggguggcggccu

SEQ ID NO: 613 - RNA, 23 nt, Homo Sapiens
aucgcugcgguugcgagcgcugu

SEQ ID NO: 614 - RNA, 21 nt, Homo Sapiens
augauccaggaaccugccucu

SEQ ID NO: 615 - RNA, 24 nt, Homo Sapiens
aaagacauaggauagagucaccuc

SEQ ID NO: 616 - RNA, 22 nt, Homo Sapiens
gucccucuccaaaugugucuug

SEQ ID NO: 617 - RNA, 22 nt, Homo Sapiens
acuuguaugcuagcucagguag

SEQ ID NO: 618 - RNA, 19 nt, Homo Sapiens
aguguggcuuucuuagagc

SEQ ID NO: 619 - RNA, 19 nt, Homo Sapiens
ucuaggcugguacugcuga

SEQ ID NO: 620 - RNA, 19 nt, Homo Sapiens
aagcagcugccucugaggc

SEQ ID NO: 621 - RNA, 21 nt, Homo Sapiens
guggcugcacucacuuccuuc

SEQ ID NO: 622 - RNA, 19 nt, Homo Sapiens
aagugugcagggcacuggu

SEQ ID NO: 623 - RNA, 22 nt, Homo Sapiens
aaaccuguguuguucaagaguc

SEQ ID NO: 624 - RNA, 21 nt, Homo Sapiens
aggaggcagcgcucucaggac

SEQ ID NO: 625 - RNA, 22 nt, Homo Sapiens
uuuaggauaagcuugacuuuug

SEQ ID NO: 626 - RNA, 21 nt, Homo Sapiens
aauggcgccacuaggguugug

SEQ ID NO: 627 - RNA, 21 nt, Homo Sapiens
guguugaaacaaucucuacug

SEQ ID NO: 628 - RNA, 22 nt, Homo Sapiens
uaugucugcugaccaucaccuu

SEQ ID NO: 629 - RNA, 22 nt, Homo Sapiens
uggugggccgcagaacaugugc

SEQ ID NO: 630 - RNA, 22 nt, Homo Sapiens
auaauacaugguuaaccucuuu

SEQ ID NO: 631 - RNA, 21 nt, Homo Sapiens
aauauuauacagucaaccucu

SEQ ID NO: 632 - RNA, 23 nt, Homo Sapiens
ggcagguucucacccucucuagg

SEQ ID NO: 633 - RNA, 25 nt, Homo Sapiens
ggcggagggaaguagguccguuggu

SEQ ID NO: 634 - RNA, 22 nt, Homo Sapiens
cuugguucagggagggucccca

SEQ ID NO: 635 - RNA, 22 nt, Homo Sapiens
uacccauugcauaucggaguug

SEQ ID NO: 636 - RNA, 24 nt, Homo Sapiens
ugccugggucucuggccugcgcgu

SEQ ID NO: 637 - RNA, 21 nt, Homo Sapiens
ucccacguuguggcccagcag

SEQ ID NO: 638 - RNA, 22 nt, Homo Sapiens
aggcggggcgccgcgggaccgc

SEQ ID NO: 639 - RNA, 20 nt, Homo Sapiens
accaggaggcugaggccccu

SEQ ID NO: 640 - RNA, 23 nt, Homo Sapiens
ugucacucggcucggcccacuac

SEQ ID NO: 641 - RNA, 21 nt, Homo Sapiens
uccgguucucagggcuccacc

SEQ ID NO: 642 - RNA, 23 nt, Homo Sapiens
aggaagcccuggaggggcuggag

SEQ ID NO: 643 - RNA, 23 nt, Homo Sapiens
ugagguugguguacuguguguga

SEQ ID NO: 644 - RNA, 22 nt, Homo Sapiens
gcacugagaugggaguggugua

SEQ ID NO: 645 - RNA, 23 nt, Homo Sapiens
uggugcggagagggcccacagug

SEQ ID NO: 646 - RNA, 23 nt, Homo Sapiens
uggaagacuagugauuuuguugu

SEQ ID NO: 647 - RNA, 23 nt, Homo Sapiens
aaggagcuuacaaucuagcuggg

SEQ ID NO: 648 - RNA, 22 nt, Homo Sapiens
caacuagacugugagcuucuag

SEQ ID NO: 649 - RNA, 22 nt, Homo Sapiens
caacaaaucacagucugccaua

SEQ ID NO: 650 - RNA, 22 nt, Homo Sapiens
caacaaaucccagucuaccuaa

SEQ ID NO: 651 - RNA, 22 nt, Homo Sapiens
ugcggggcuagggcuaacagca

SEQ ID NO: 652 - RNA, 22 nt, Homo Sapiens
cuguugccacuaaccucaaccu

SEQ ID NO: 653 - RNA, 22 nt, Homo Sapiens
uuugugaccugguccacuaacc

SEQ ID NO: 654 - RNA, 20 nt, Homo Sapiens
cggcucugggucugugggga

SEQ ID NO: 655 - RNA, 21 nt, Homo Sapiens
uggaggagaaggaaggugaug

SEQ ID NO: 656 - RNA, 22 nt, Homo Sapiens
acuccagccccacagccucagc

SEQ ID NO: 657 - RNA, 23 nt, Homo Sapiens
ucugcucauaccccaugguuucu

SEQ ID NO: 658 - RNA, 23 nt, Homo Sapiens
ugcaccaugguugucugagcaug

SEQ ID NO: 659 - RNA, 28 nt, Homo Sapiens
ucacaaugcugacacucaaacugcugac

SEQ ID NO: 660 - RNA, 26 nt, Homo Sapiens
guuggaggaugaaaguacggagugau

SEQ ID NO: 661 - RNA, 23 nt, Homo Sapiens
cugggaucuccggggucuugguu

SEQ ID NO: 662 - RNA, 22 nt, Homo Sapiens
ugagaccucuggguucugagcu

SEQ ID NO: 663 - RNA, 23 nt, Homo Sapiens
uccaguaccacgugucagggcca

SEQ ID NO: 664 - RNA, 24 nt, Homo Sapiens
gauugcucugcgugcggaaucgac

SEQ ID NO: 665 - RNA, 23 nt, Homo Sapiens
caguaacaaagauucauccuugu

SEQ ID NO: 666 - RNA, 23 nt, Homo Sapiens
uauucagauuagugccagucaug

SEQ ID NO: 667 - RNA, 21 nt, Homo Sapiens
aagguuacuuguuaguucagg

SEQ ID NO: 668 - RNA, 21 nt, Homo Sapiens
gcaggaacuugugagucuccu

SEQ ID NO: 669 - RNA, 22 nt, Homo Sapiens
cugcccuggcccgagggaccga

SEQ ID NO: 670 - RNA, 21 nt, Homo Sapiens
ccuggaaacacugagguugug

SEQ ID NO: 671 - RNA, 22 nt, Homo Sapiens
uauaccucaguuuuaucaggug

SEQ ID NO: 672 - RNA, 22 nt, Homo Sapiens
uggugguuuacaaaguaauuca

SEQ ID NO: 673 - RNA, 22 nt, Homo Sapiens
uggauuucuuugugaaucacca

SEQ ID NO: 674 - RNA, 20 nt, Homo Sapiens
guagaggagauggcgcaggg

SEQ ID NO: 675 - RNA, 22 nt, Homo Sapiens
uccucuucucccuccucccagg

SEQ ID NO: 676 - RNA, 22 nt, Homo Sapiens
aggcagcgggguguaguggaua

SEQ ID NO: 677 - RNA, 22 nt, Homo Sapiens
uccauuacacuacccugccucu

SEQ ID NO: 678 - RNA, 21 nt, Homo Sapiens
cgcgggugcuuacugacccuu

SEQ ID NO: 679 - RNA, 23 nt, Homo Sapiens
cgggucggaguuagcucaagcgg

SEQ ID NO: 680 - RNA, 22 nt, Homo Sapiens
gugaacgggcgccaucccgagg

SEQ ID NO: 681 - RNA, 21 nt, Homo Sapiens
uacucaaaaagcugucaguca

SEQ ID NO: 682 - RNA, 22 nt, Homo Sapiens
gacugacaccucuuugggugaa

SEQ ID NO: 683 - RNA, 21 nt, Homo Sapiens
uuaauaucggacaaccauugu

SEQ ID NO: 684 - RNA, 21 nt, Homo Sapiens
uacuuggaaaggcaucaguug

SEQ ID NO: 685 - RNA, 22 nt, Homo Sapiens
ugcaacgaaccugagccacuga

SEQ ID NO: 686 - RNA, 22 nt, Homo Sapiens
ugcaacuuaccugagucauuga

SEQ ID NO: 687 - RNA, 21 nt, Homo Sapiens
cacuguguccuuucugcguag

SEQ ID NO: 688 - RNA, 22 nt, Homo Sapiens
cacuggcuccuuucuggguaga

SEQ ID NO: 689 - RNA, 23 nt, Homo Sapiens
ucuuugguuaucuagcuguauga

SEQ ID NO: 690 - RNA, 22 nt, Homo Sapiens
auaaagcuagauaaccgaaagu

SEQ ID NO: 691 - RNA, 20 nt, Homo Sapiens
ggggagcuguggaagcagua

SEQ ID NO: 692 - RNA, 25 nt, Homo Sapiens
cuagugagggacagaaccaggauuc

SEQ ID NO: 693 - RNA, 23 nt, Homo Sapiens
gcagcagagaauaggacuacguc

SEQ ID NO: 694 - RNA, 21 nt, Homo Sapiens
gucagcggaggaaaagaaacu

SEQ ID NO: 695 - RNA, 20 nt, Homo Sapiens
agagucuugugaugucuugc

SEQ ID NO: 696 - RNA, 22 nt, Homo Sapiens
uauugcacuugucccggccugu

SEQ ID NO: 697 - RNA, 23 nt, Homo Sapiens
agguugggaucgguugcaaugcu

SEQ ID NO: 698 - RNA, 22 nt, Homo Sapiens
ggguggggauuuguugcauuac

SEQ ID NO: 699 - RNA, 22 nt, Homo Sapiens
uauugcacucgucccggccucc

SEQ ID NO: 700 - RNA, 22 nt, Homo Sapiens
agggacgggacgcggugcagug

SEQ ID NO: 701 - RNA, 23 nt, Homo Sapiens
caaagugcuguucgugcagguag

SEQ ID NO: 702 - RNA, 22 nt, Homo Sapiens
acugcugagcuagcacuucccg

SEQ ID NO: 703 - RNA, 22 nt, Homo Sapiens
ugugcgcagggagaccucuccc

SEQ ID NO: 704 - RNA, 22 nt, Homo Sapiens
ugucuacuacuggagacacugg

SEQ ID NO: 705 - RNA, 23 nt, Homo Sapiens
ccaguuaccgcuuccgcuaccgc

SEQ ID NO: 706 - RNA, 22 nt, Homo Sapiens
acaguagagggaggaaucgcag

SEQ ID NO: 707 - RNA, 22 nt, Homo Sapiens
auccgcgcucugacucucugcc

SEQ ID NO: 708 - RNA, 22 nt, Homo Sapiens
ugcccuuaaaggugaacccagu

SEQ ID NO: 709 - RNA, 24 nt, Homo Sapiens
uggggagcugaggcucugggggug

SEQ ID NO: 710 - RNA, 21 nt, Homo Sapiens
aaggcagggcccccgcucccc

SEQ ID NO: 711 - RNA, 23 nt, Homo Sapiens
cacccggcugugugcacaugugc

SEQ ID NO: 712 - RNA, 22 nt, Homo Sapiens
ucuucucuguuuuggccaugug

SEQ ID NO: 713 - RNA, 21 nt, Homo Sapiens
cugacuguugccguccuccag

SEQ ID NO: 714 - RNA, 22 nt, Homo Sapiens
aaauuauuguacaucggaugag

SEQ ID NO: 715 - RNA, 22 nt, Homo Sapiens
uucaacggguauuuauugagca

SEQ ID NO: 716 - RNA, 23 nt, Homo Sapiens
uuuggcacuagcacauuuuugcu

SEQ ID NO: 717 - RNA, 22 nt, Homo Sapiens
aaucaugugcagugccaauaug

SEQ ID NO: 718 - RNA, 22 nt, Homo Sapiens
ugagguaguaaguuguauuguu

SEQ ID NO: 719 - RNA, 22 nt, Homo Sapiens
aacccguagauccgaucuugug

SEQ ID NO: 720 - RNA, 22 nt, Homo Sapiens
caagcucgcuucuaugggucug

SEQ ID NO: 721 - RNA, 22 nt, Homo Sapiens
cacccguagaaccgaccuugcg

SEQ ID NO: 722 - RNA, 22 nt, Homo Sapiens
caagcucgugucuguggguccg

SEQ ID NO: 723 - RNA, 20 nt, Herpes Simplex
uggcggcccggcccggggcc

(19) United States
(12) Patent Application Publication
(10) Pub. No.: US 2010/0021914 A1
(43) Pub. Date: Jan. 28, 2010

(54) OLIGONUCLEOTIDES FOR MODULATING TARGET RNA ACTIVITY

(75) Inventor: Thorleif Møller, Linhamm (SE)

Correspondence Address: FOLEY AND LARDNER LLP;SUITE 500, 3000 K STREET NW, WASHINGTON, DC, 20007, US

(73) Assignee: QUERDENKER APS

(21) Appl. No.: 12/516,205

(22) PCT Filed: Nov. 23, 2007
(86) PCT No.: PCT/DK07/50174

§ 371 Date: Aug. 19, 2009

Related U.S. Application Data
(60) Provisional application No. 60/888,095, filed on Feb. 4, 2007.
(60) Provisional application No. 60/888,094, filed on Feb. 4, 2007.

(30) Foreign Application Priority Data
Nov. 23, 2006 (DK) PA 2006 01543
Nov. 23, 2006 (DK) PA 2006 01544
Jul. 24, 2007 (DK) PA 2007 01081

(51) Int. Cl. C12Q 1/68; C07H 21/02

(52) U.S. Cl. 435/6; 536/23.1

(57) ABSTRACT

The present invention describes oligonucleotides that bind to microRNA target sites in target RNAs, such as mRNAs. The oligonucleotides of the invention may mediate RNase H degradation of the target RNA, mediate RNAi of the target RNA or prevent microRNA regulation of the target RNA. The oligonucleotides of the invention are useful e.g. as research tools for studying microRNA:mRNA interactions and for therapeutic development. The present invention also describes methods of identifying microRNA target sites, methods of validating microRNA target sites, methods of identifying oligonucleotides of the invention and methods of modulating the activity of a target RNA using the oligonucleotides of the invention.

### BACKGROUND OF THE INVENTION

The present invention relates to oligonucleotides that can be used to affect the activity of target RNAs.

The first generation of such oligonucleotides were antisense oligonucleotides that were intended to affect the activity of target mRNAs. One reason for interest in such oligonucleotides is the potential for exquisite and predictable specificity that can be achieved because of specific base pairing. In other words, it is in theory very simple to design an oligonucleotide that is highly specific for a given nucleic acid, such as an mRNA.

However, it has turned out that not all sequences are available for antisense targeting and accessibility may vary e.g. because of secondary structure or protein binding.

Moreover, it has turned out simple base pairing is not enough to achieve regulation of a given target mRNA, i.e. an oligonucleotide complementary to a given target mRNA does not necessarily affect the activity of the target mRNA. If the oligonucleotide targets the open reading frame of an mRNA, it may e.g. be that the translational apparatus simply displaces the oligonucleotide during translation. Therefore, means where developed that would improve the regulatory activity of the oligonucleotide.

E.g. oligonucleotides that can activate RNase H cleavage of the target mRNA were developed. One potential disadvantage of such oligonucleotides is that they may mediate cleavage of other RNAs than the intended target mRNA, i.e. giving rise to off-target effects. Still, oligonucleotides acting through RNase H cleavage are in clinical trials for treatment of various diseases.

Recently, research has shown that eukaryotic cells, including mammalian cells, comprise a complex gene regulatory system (herein also termed RNAi machinery) that uses RNA as specificity determinants. This system can be triggered by so called siRNAs that may be introduced into a cell of interest to regulate the activity of a target mRNA. Currently, massive efforts go into triggering the RNAi machinery with siRNAs for specific regulation of target RNAs, in particular target mRNAs. This approach is widely regarded as having great promise for the development of new therapeutics. As will also be outlined below, a major advantage of this approach is that specificity of the siRNA lies in the degree of complementarity between the guide strand of the siRNA and the target RNA, i.e. target specificity can be controlled. However, it has turned out that siRNAs may be less specific than initially thought. Initially, it was believed that only target RNAs that harboured stretches of complete complementarity to the guide strand of the siRNA would be affected, i.e. targeted by the RNAi machinery. New research indicates that siRNAs indeed do result in significant off-target effects, i.e. regulation of non-intended targets. It is now believed that these off-targets stem from the siRNAs, or rather the guide strand of the siRNAs, acting as microRNAs.

MicroRNAs are a class of endogenous RNA molecules that has recently been discovered and that, as siRNA, function via the RNAi machinery. Currently, about 500 human microRNAs have been discovered and the number is rapidly increasing. It is now believed that more than one third of all human genes may be regulated by microRNAs. Therefore, microRNAs themselves may be used to regulate the activity of target RNAs, and consequently e.g. be used as therapeutics.

However, as also described below, microRNAs generally act at more than one target RNA, i.e. they are promiscuous. Thus, introduction of a microRNA into the cell or regulating the level of a microRNA will affect the activity of more than one target mRNA and consequently may give rise to undesired off-target effects.

A recent approach has been put forward, wherein the activity of a target RNA is regulated by inhibiting the activity of a microRNA. The microRNA can be inhibited using complementary oligonucleotides that have been termed antimirs and antagomirs. Since the microRNA is itself promiscuous, also an antimir or antagomir will be promiscuous and affect the activity of more than one target RNA.

### DETAILED DESCRIPTION

In previous applications (PA 2006 01543 and PA 2006 01544 filed in Denmark, November 23, and U.S. 60,888,094 and U.S. 60/888,095 filed Feb. 4, 2007 in the US) the term Xmir was used, when referring to oligonucleotides of the invention. In this application, the term oligonucleotides of the invention are preferentially used over the term Xmir. However, when the term Xmir is used, reference is to oligonucleotides of the invention.

Thus, as used herein, the term Xmir refers to an oligonucleotide of the invention as specified further in the following embodiments and in the claims.

All references mentioned herein are hereby incorporated by reference.

It is to be understood that features described in one aspect of the invention equally applies for the other aspects.

### DEFINITIONS

An “oligonucleotide capable of regulating the activity of a target RNA” refers to an oligonucleotide with a particular activity. Such oligonucleotides are also termed active oligonucleotides.

The terms “regulate” and “modulate” are used interchangeably herein.

An “oligonucleotide potentially capable of regulating the activity of a target mRNA” refers to an oligonucleotide which activity has not yet been experimentally confirmed. Such an oligonucleotide may also be termed a candidate regulator.

When reference is made to an oligonucleotide without further specification, the oligonucleotide may be an “oligonucleotide capable of regulating the activity of an mRNA” or an “oligonucleotide potentially capable of regulating the activity of a target mRNA” or both.

When referring to a “target RNA”, what is meant is the target for an oligonucleotide of the invention. Typically, an oligonucleotide of the invention can interact with a target RNA by way of base pairing.

The target RNA may be any RNA. Preferably, the target RNA is a mRNA or a viral RNA, such as a genomic viral RNA.

When referring to the “activity of a target mRNA”, what is typically meant is the expression of the target mRNA, i.e. translation into a protein or peptide. Thus, regulation of the activity of a target mRNA may include degradation of the mRNA and/or translational regulation. Regulation may also include affecting intracellular transport of the mRNA. In a preferred embodiment of the invention, the oligonucleotide is capable of regulating the expression of the target mRNA. In another preferred embodiment, the oligonucleotide may mediate degradation of the target mRNA (in turn also regulating expression of the target mRNA). The activity may also be replication.

When the target RNA is a viral RNA, the oligonucleotide of the invention may affect replication of the virus or otherwise interfere with the proliferation of the virus.

As used herein, regulation may be either positive or negative. I.e. a regulator (e.g. oligonucleotide or microRNA) may increase the activity of the target (e.g. target mRNA) or it may decrease the activity of the target.

When referring to the “target sequence of an RNA”, what is meant is the region of the RNA involved in or necessary for microRNA regulation. The terms “target region” and “target sequence” are used interchangeably herein.

Not intended to be bound by theory, it is believed that this region comprise bases that interact directly with the microRNA during microRNA regulation of the target RNA. In a preferred embodiment, the target sequence is the region of the target RNA necessary for microRNA regulation. Such region may be defined using a reporter system, wherein systematic deletions of the target RNA are tested for activity to define the target sequence. Assessing the effect of introducing point mutations in the target region is also valuable for defining the target region.

As will be clear from the specification, also oligonucleotides of the invention may be used to define the region of the target RNA necessary for microRNA regulation. Preferably, the target sequence comprises an antiseed sequence, which is complementary to the seed sequence of a microRNA and also complementary to a guide sequence of a oligonucleotide of the invention. Introduction of mutations in the antiseed sequence will typically affect microRNA regulation and hence may be used to verify that given positions are involved in microRNA regulation.

The term microRNA as used herein has the same meaning as typically in the art. I.e. the term microRNA refers to a small non-translated RNA of typically 18-22 nucleotides that is capable of regulating the activity of a target mRNA. A microRNA is typically processed from pri-microRNA to short stem-loop structures called pre-microRNA and finally to mature miRNA. Both strands of the stem of the pre-microRNA may be processed to a mature microRNA.

The miRBase (http://microrna.sanger.ac.uk/sequences/) is a compilation of known microRNAs. Also predicted and known targets of the microRNAs can be found on this site.

The term siRNA (short interfering RNA) as used herein has the same meaning as typically in the art. I.e. the term siRNA refers to double stranded RNA complex wherein the strands are typically 18-22 nucleotides in length. Very often, the complex has 3′-overhangs.

When referring to the RNAi machinery herein, what is meant are the cellular components necessary for the activity of siRNAs and microRNAs or for the RNAi pathway. A major player of the RNAi machinery is the RNA induced silencing complex (the RISC complex).

As referred to herein, a RNA unit is one of the monomers that make up an RNA polymer. Thus, an RNA unit is also referred to as an RNA monomer or a RNA nucleotide. Likewise, a DNA unit is one of the monomers that make up a DNA polymer and a DNA unit may also be referred to as a DNA monomer or a DNA nucleotide.

When referring to a base, what is meant is the base of a nucleotide. The base may be part of DNA, RNA, INA, LNA or any other nucleic acid or nucleic acid capable of specific base pairing. The base may also be part of PNA (peptide nucleic acid). In some embodiments, the base may be an universal base.

When referring the length of a sequence, reference may be made to the number of units or to the number of bases.

When referring to a complementary sequence, G pairs to C, A pairs to T and U and vice versa. In a preferred embodiment, G also pairs to U and vice versa to form a so-called wobble base pair. In another preferred embodiment, the base inosine (I) may be comprised within either in a microRNA or oligonucleotide of the invention. I basepairs to A, C and U. In still another preferred embodiment, universal bases may be used. Universal bases can typically basepair to G, C, A, U and T. Often universal bases do not form hydrogen bonds with the opposing base on the other strand. In still another preferred embodiment, a complementary sequence refers to a contiguous sequence exclusively of Watson-Crick base pairs.

### SUMMARY OF THE INVENTION

In a first aspect, the present invention provides oligonucleotides that are useful for modulating the activity of a target RNA. In a preferred embodiment, the oligonucleotides target a microRNA target region of the target RNA. Another aspect of the invention is a method for modulating the activity of a target RNA. Still other aspects relate to providing an oligonucleotide of the invention, identifying microRNA target regions of target RNAs, validating microRNA target regions of target RNAs and identifying microRNA regulators of a given target RNA.

### DISCLOSURE OF THE INVENTION

The present invention provides oligonucleotides that target microRNA target regions of target RNAs. In one embodiment, the oligonucleotides draws use of the accessibility of microRNA target regions of target RNAs. The oligonucleotides of the invention may recruit the RNAi machinery to the target RNA to mediate translational repression or cleavage of the target RNA. The oligonucleotides of the invention may also recruit RNase H to mediate cleavage of the target RNA. Moreover, the oligonucleotides of the invention may modulate the activity of the target RNA by preventing a microRNA from regulating the target RNA. The invention also provides methods for providing microRNA targets of target RNAs, methods for validating microRNA target regions of target RNAs and methods of modulating the activity of target RNAs using oligonucleotides of the invention.

### First Aspect—Bioactive Oligonucleotides

In a first aspect, the present invention provides an oligonucleotide comprising an antisense sequence that comprises a guide sequence corresponding to the seed sequence of a microRNA, with the proviso that the oligonucleotide is not a microRNA or does not comprise a sequence corresponding the complete sequence of a microRNA.

Such an oligonucleotide is of interest because it can be used to target the target region of a target RNA, said target region being involved in microRNA regulation of the target RNA. Not intended to be bound by theory, it is believed that said target region will be more accessible for interaction (with microRNAs, oligonucleotides or other nucleic acids) than will other regions of the target RNA, because the target region is evolved for interaction with a microRNA or because endogenous microRNAs chooses target regions that are more accessible.

Support for the above view comes from work published after the priority date of this patent application. One publication investigated the effect of target secondary structure on the efficacy of repression by microRNAs (Long D, 2007). The results indicate a potent effect of target structure on target recognition by microRNAs, at least for microRNA regulation in Caenorhabditis elegans and Drosophila melanogaster. The authors suggest that target secondary structure probably contributes to accessibility in most miRNA-target interactions.

Another study systematically investigated the role of target-site accessibility in microRNA target recognition (Kertesz M, 2007). The authors demonstrated that mutations diminishing target accessibility substantially reduce microRNA mediated translational repression. Moreover, the authors performed a genome-wide analysis of target accessibility to all 3′UTRs of fly, worm, mouse and human. They found that microRNA seed sequences in all four organisms showed a notable preference for highly accessible regions and the authors suggest that target accessibility is a critical factor in microRNA function.

We suggest that target accessibility will most likely be determined by a combination of target secondary structure and occlusion by other factors such as RNA binding proteins.

Thus, in one embodiment of the present invention, the target region may be targeted by e.g. RNase H inducing oligonucleotides or siRNAs. The oligonucleotide may e.g. be a 10-mer that induces RNase H cleavage of the target RNA. The oligonucleotide may also prevent a microRNA from exerting its action on the target RNA. These various embodiments will be further outlined below.

As used in the context of the guide sequence (of an oligonucleotide of the invention) and the seed sequence (of a microRNA), the word “corresponding” refers to the ability of the seed sequence and the guide sequence of being capable of base pairing with the same sequence. I.e. the guide sequence and the seed sequence may not necessarily be identical, but they are capable of base pairing to the same sequence, e.g. the anti-seed sequence of a target RNA.

The phrase “a sequence corresponding to the complete sequence of a microRNA sequence” is intended to cover e.g. a precursor of the microRNA or a DNA molecule that encode the microRNA. The DNA molecule may e.g. be a PCR product intended for T7 RNA polymerase transcription of the microRNA. Such molecules are not included in the scope of the invention, as neither are naturally occurring microRNAs.

### Origin

The target RNAs to be used in the methods of the present invention are preferably of animal or plant origin. More preferably, the target RNAs are of mammalian origin. Most preferably they are of human origin. The target RNAs may also be of viral origin, preferably from virus that infects humans. In a preferred embodiment, the term human target RNA also include viral target RNAs of virus that infects humans.

The microRNAs to be used in the methods of the present invention are also preferably of animal or plant origin. More preferably, they are of mammalian origin. Most preferably, they are of human origin. The microRNAs to be used in the methods of the present invention may also be of viral origin. If they are of viral origin, they are preferably from virus that infects humans, e.g. mir-LAT of HSV-1. In a preferred embodiment, the term human microRNAs also include viral microRNAs of virus that infect humans.

It is most preferred that the oligonucleotides of the invention comprise a guide sequence that corresponds to the seed sequence of a human microRNA or of a microRNA from a virus that infects humans.

In a preferred embodiment, the oligonucleotide of the invention comprise a sequence selected from the group consisting of sequences that are capable of base pairing to the complementary sequence of a sequence selected from the group consisting of position 1-20, position 1-19, position 1-18, position 1-17, position 1-16, position 1-15, position 1-14, position 1-13, position 1-12, position 1-11, position 1-10, position 1-9, position 1-8, position 1-7, position 1-6, position 2-20, position 2-19, position 2-18, position 2-17, position 2-16, position 2-15, position 2-14, position 2-13, position 2-12, position 2-11, position 2-10, position 2-9, position 2-8, position 2-7, position 2-6, position 3-20, position 3-19, position 3-18, position 3-17, position 3-16, position 3-15, position 3-14, position 3-13, position 3-12, position 3-11, position 3-10 and position 3-9 of any SEQ ID NOs:1-723.

In a preferred embodiment, the oligonucleotide of the invention comprise a sequence selected from the group consisting of sequences that are capable of base pairing to the complementary sequence of a sequence selected from the group consisting of position 1-20, position 1-19, position 1-18, position 1-17, position 1-16, position 1-15, position 1-14, position 1-13, position 1-12, position 1-11, position 1-10, position 1-9, position 1-8, position 1-7, position 1-6, position 2-20, position 2-19, position 2-18, position 2-17, position 2-16, position 2-15, position 2-14, position 2-13, position 2-12, position 2-11, position 2-10, position 2-9, position 2-8, position 2-7, position 2-6, position 3-20, position 3-19, position 3-18, position 3-17, position 3-16, position 3-15, position 3-14, position 3-13, position 3-12, position 3-11, position 3-10 and position 3-9 of any SEQ ID NOs:1-723 and are not capable of forming a consecutive base pair with the neighbouring nucleotide of either side of the aforementioned positions.

The term complementary sequence has been defined above. The phrase “are capable of base pairing to” is related to the term complementary sequence. I.e. a first sequence is capable of base pairing to a second sequence, which is complementary to the first sequence.

In another preferred embodiment, the oligonucleotide of the invention consists of an antisense sequence selected from the group consisting of sequences that are capable of base pairing to the complementary sequence of a sequence selected from the group consisting selected from the group consisting of position 1-20, position 1-19, position 1-18, position 1-17, position 1-16, position 1-15, position 1-14, position 1-13, position 1-12, position 1-11, position 1-10, position 1-9, position 1-8, position 1-7, position 1-6, position 2-20, position 2-19, position 2-18, position 2-17, position 2-16, position 2-15, position 2-14, position 2-13, position 2-12, position 2-11, position 2-10, position 2-9, position 2-8, position 2-7, position 2-6, position 3-20, position 3-19, position 3-18, position 3-17, position 3-16, position 3-15, position 3-14, position 3-13, position 3-12, position 3-11, position 3-10 and position 3-9 of any SEQ ID NOs:1-723.

The oligonucleotides of the invention can also be defined by base pairing rules. Thus, in another preferred embodiment, the antisense sequence of the oligonucleotides of the invention comprises an sequence selected from the group consisting of position 1-20, position 1-19, position 1-18, position 1-17, position 1-16, position 1-15, position 1-14, position 1-13, position 1-12, position 1-11, position 1-10, position 1-9, position 1-8, position 1-7, position 1-6, position 2-20, position 2-19, position 2-18, position 2-17, position 2-16, position 2-15, position 2-14, position 2-13, position 2-12, position 2-11, position 2-10, position 2-9, position 2-8, position 2-7, position 2-6, position 3-20, position 3-19, position 3-18, position 3-17, position 3-16, position 3-15, position 3-14, position 3-13, position 3-12, position 3-11, position 3-10 and position 3-9 of any SEQ ID NOs:1-723, wherein
a. A may be exchanged with only G, C, U, T or I
b. G may be exchanged with only A or I
c. C may be exchanged with only A, U or T
d. U may be exchanged with only C, A, T or I
and 3 additional positions may be exchanged with any base.

The exchange rules are based on the following considerations:

An A in the microRNA can base pair to U or I in the target RNA. U and I in the target RNA can base pair to A, G, I, C, U or T. Likewise for the other bases.

Moreover, editing of A to I in microRNAs has been shown to redirect silencing targets of microRNAs (Kawahara Y, 2007). Therefore, A in the microRNAs may be substituted for 1 some embodiments.

Also the target RNA may comprise I that have been edited from A.

Moreover, G:U base pairs may be accepted for microRNAs—target RNA interaction in some embodiments, but not all.

The rules are described in table 1:

| MicroRNA | | | | | |
|---|---|---|---|---|---|
| U | G | C | A | I | A/I |

Inosines in target RNA and miRNA + GU basepairs

| | | | | | | |
|---|---|---|---|---|---|---|
| target | A, G, I | U, C | G, I | U, I | A, C, U | |
| RNA | | | | | | |
| Xmir | U, I, A, | A, G, I | U, C, A, T | A, G, I, C, | U, I, A, | A, G, |
| | C, T | | | U, T | G, T | I, C, |
| | | | | | | U, T |

Inosines in target RNA and miRNA + GU pairs, no T-I
pairs

| | | | | | | |
|---|---|---|---|---|---|---|
| target | A, G, I | U, C | G, I | U, I | A, C, U | |
| RNA | | | | | | |
| Xmir | U, I, A, | A, G, I | U, C, A | A, G, I, C, U | U, I, A, | A, G, |
| | C, T | | | | G, T | I, C, |
| | | | | | | U, T |

Inosines in target RNA and miRNA, no GU basepairs

| | | | | | | |
|---|---|---|---|---|---|---|
| target | A, I | C | G, I | U, I | A, C, U | |
| RNA | | | | | | |
| Xmir | U, I, A, | G, I | A, C, U, T | A, I, C, U, T | U, I, G, | A, G, |
| | C, T | | | | A, T | I, C, |
| | | | | | | U, T |

Inosines in target RNA and miRNA, no GU pairs, no I-T
pairs

| | | | | | | |
|---|---|---|---|---|---|---|
| target | A, I | C | G, I | U, I | A, C, U | |
| RNA | | | | | | |
| Xmir | U, I, A, | G, I | A, C, U | A, I, C, U | U, I, G, | A, G, |
| | C, T | | | | A, T | I, C, |
| | | | | | | U, T |

No inosine in target RNA

| | | | | | | |
|---|---|---|---|---|---|---|
| target | A, G | U, C | G, I | U | A, C, U | |
| RNA | | | | | | |
| Xmir | U, C, T | A, G, I | U, C, A, T | A, G, I | U, G, I, | U, G, |
| | | | | | A, T | I, A, T |

MicroRNA

| | | | |
|---|---|---|---|
| U | G | C | A |

No inosine in either target RNA or
miRNA

| | | | | |
|---|---|---|---|---|
| target | A, G | U, C | G | U |
| RNA | | | | |
| Xmir | U, C, T | A, G | U, C, T | A, G |

No GU pairs and no inosine in either target RNA or
miRNA

| | | | | |
|---|---|---|---|---|
| target | A | C | G | U |
| RNA | | | | |
| Xmir | U, T | G | C | A |

Additional positions that may be exchanged with any base are included to account for single nucleotide polymorphisms (SNPs) and other mutations. Furthermore, some target sequences interacting with microRNAs may not posses' perfect complementarity to the interacting microRNA. I.e. there may be a mismatch in the complex formed between the seed sequence of the microRNA and the antiseed sequence of the target RNA.

Thus, in another preferred embodiment,
a. A may be exchanged with only G, C, U, T or I
b. G may be exchanged with only A or I
c. C may be exchanged with only A or U
d. U may be exchanged with only C, A, T or I
and 3 additional positions may be exchanged with any base.

In yet another preferred embodiment,
a. A may be exchanged with only C, U, T or I
b. G may be exchanged with only I
c. C may be exchanged with only A, U or T
d. U may be exchanged with only C, A, T or I
and 3 additional positions may be exchanged with any base.

In yet another preferred embodiment,
a. A may be exchanged with only C, U, or I
b. G may be exchanged with only I
c. C may be exchanged with only A or U
d. U may be exchanged with only C, A, T or I
and 3 additional positions may be exchanged with any base.

In yet another preferred embodiment,
a. A may be exchanged with only G or I
b. G may be exchanged with only I or A
c. C may be exchanged with only A, U or T
d. U may be exchanged with only C or T
and 3 additional positions may be exchanged with any base.

In yet another preferred embodiment,
a. A may be exchanged with only G
b. G may be exchanged with only A or G
c. C may be exchanged with only T or U
d. U may be exchanged with only C or T
and 3 additional positions may be exchanged with any base.

In yet another preferred embodiment, U may be exchanged with only T
and 3 additional positions may be exchanged with any base.

In yet another preferred embodiment, 2 additional positions may be exchanged with any base.

In yet another preferred embodiment, 1 additional position may be exchanged with any base.

In yet another preferred embodiment, no additional positions may be exchanged with any base.

In a preferred embodiment, the oligonucleotide may further comprise 1 or 2 additions or deletions. More preferred is 1 addition/substitution and most preferred is zero additions/deletions. Additions and deletions are relevant where the complex between the microRNA and target RNA comprise bulges. If a nucleotide on the microRNA is bulged, this accounts to a deletion of the oligonucleotide of the invention. If a nucleotide on the target RNA is bulged, this accounts for a addition of the oligonucleotide of the invention.

It is even more preferred that the oligonucleotide of the invention comprise an antisense sequence that comprises a guide sequence selected from the group consisting of: position 1-10, position 1-9, position 1-8, position 1-7, position 1-6, position 2-10, position 2-9, position 2-8, position 2-7, position 2-6, position 3-10 and position 3-9 of any SEQ ID NOs: 1-723 wherein it is to be understood that the exchange rules outlined above also apply for this group, i.e. in various embodiments.

It is most preferred that the oligonucleotide of the invention comprise an antisense sequence that comprises a guide sequence selected from the group consisting of: position 1-8, position 1-7, position 2-8 and position 2-7 of any SEQ ID NOs: 1-723 wherein it is to be understood that the exchange rules outlined above also apply for this group, i.e. in various embodiments.

In one embodiment, the oligonucleotide does not comprise the neighbouring nucleotide of either side of the aforementioned positions of any of SEQ ID NOs 1-723. I.e. the neighbouring positions of any of the aforementioned positions of any of SEQ ID NOs 1-723 are not the same as the corresponding neighbouring positions of the oligonucleotides of the invention.

It still another preferred embodiment, the oligonucleotide of the invention consists of an antisense sequence comprises a guide sequence selected from the group consisting of: position 1-8, position 1-7, position 2-8 and position 2-7 of any of SEQ ID NOs:1-723 wherein it is to be understood that the exchange rules outlined above also apply for this group, i.e. in various embodiments.

### Second Sequence

In another preferred embodiment, the antisense sequence of the oligonucleotide of the invention further comprises a second sequence selected from the group consisting of: position 12-17, position 12-16, position 13-17 and position 13-16 of any of SEQ ID NOs: 1-723, wherein the guide sequence and the second sequence are derived from the same SEQ ID NO and wherein it is to be understood that the exchange rules outlined above also apply for this group, i.e. in various embodiments.

### Contiguous Stretch of Bases

Preferably, the oligonucleotide of the invention comprises a antisense sequence that comprises a contiguous stretch of bases, complementary to the micro RNA target sequence of a target RNA selected from the group consisting of: less than 60 bases, less than 50 bases, less than 40 bases, less than 39 bases, less than 38 bases, less than 37 bases, less than 36 bases, less than 35, less than 34 bases, less than 33 bases, less than 32 bases, less than 31 bases, bases, less than 30 bases, less than 29 bases, less than 28 bases, less than 27 bases, less than 26 bases, less than 25 bases, less than 24 bases, less than 23 bases, less than 22 bases, less than 21 bases, less than 20 bases, less than 19 bases, less than 18 bases, less than 17 bases, less than 16 bases, less than 15 bases, less than 14 bases, less than 13 bases, less than 12 bases, less than 11 bases, less than 10 bases, less than 9 bases, less than 8 bases, less than 7 bases, more than 60 bases, more than 50 bases, more than 40 bases, more than 39 bases, more than 38 bases, more than 37 more, more than 36 bases, more than 35, more than 34 bases, more than 33 bases, more than 32 bases, more than 31, more than 30 bases, more than 29 bases, more than 28 bases, more than 27 bases, more than 26 bases, more than 25 bases, more than 24 bases, more than 23 bases, more than 22 bases, more than 21 bases, more than 20 bases, more than 19 bases, more than 18 bases, more than 17 bases, more than 16 bases, more than 15 bases, more than 14 bases, more than 13 bases, more than 12 bases, more than 11 bases, more than 10 bases, more than 9 bases, more than 8 bases, more than 7 bases, more than 6 bases and more than 5 bases.

A contiguous stretch of bases is intended to mean a non-interrupted sequence of bases that all fit into a duplex formed between the oligonucleotide of the invention and the target RNA. I.e. there are preferably no bulges in the duplex and it is preferred that the sequences are complementary (see the definition of complementary sequences above). Most preferred is perfect Watson-Crick duplex between the oligonucleotide of the invention and target region of the target RNA.

The terms contiguous and continuous are used interchangeably herein.

In another embodiment, the oligonucleotide of the invention comprise an antisense sequence that comprises a contiguous stretch of bases complementary to the micro RNA target sequence of a target RNA, said contiguous stretch of bases being selected from the group consisting of between 10 and 14 bases, between 12 and 16 bases, between 14 and 18 bases, between 16 and 20, between 10 and 25 bases, between 12 and 24 bases, between 14 and 22 bases, between 15 and 22 bases and between 15 and 20 bases.

More preferred is a contiguous stretch of bases between 8 and 25 bases.

Most preferred is a contiguous stretch of bases between 10 and 20 bases.

Preferably, the oligonucleotide can interact with the same region of the target RNA as a microRNA. One advantage of such an oligonucleotide is that it targets an exposed region of the target RNA (see discussion above). Another advantage of such an oligonucleotide is that is can be used to mask the microRNA target such that the (endogenous) microRNA targeting the target RNA will be prevented from interacting with the target RNA, and thus exerts its effects on the target RNA.

The oligonucleotide of the invention may have a degree of identity to its corresponding microRNA selected from the group consisting of less than 99%, less than 95%, less than 90%, less than 85%, less than 80%, less than 75%, less than 70%, less than 65%, less than 60%, less than 55%, less than 50%, less than 45%, less than 40%, less than 35%, less than 30% and less than 25%. When referring to the degree of identity, the degree is counted over the length of the shortest molecule of the micro RNA and the oligonucleotide of the invention. The guide sequence of the oligonucleotide of the invention and the seed sequence of the microRNA is used for alignment. Hence, if the microRNAs is 20 bases and the oligonucleotide is 14 and the number of identical positions are 12, the degree of identity is 12/14=86%. If the microRNAs is 20, the oligonucleotide 20 and the number of positions is 10, then the degree of identity is 10/20=50%.

Preferably, the position of the guide sequence within the oligonucleotide of the invention is selected from the group consisting of: position 1, position 2, position 3, position 4, position 5, position 6, position 7, position 8, position 9, position 10, position 11, position 12, position 13, position 14, position 15, position 16, position 17, position 18 and position 19, wherein the position is counted in the 5′-3′ direction from the first base of the guide sequence and the first base of the oligonucleotide.

More preferably the position is selected from the group consisting of: position 1, position 2, position 3, position 4 and position 5.

As mentioned earlier, the guide sequence corresponds to the seed sequence of a microRNA, which is defined elsewhere in the specification.

The length of the oligonucleotide of the invention may be adjusted for various purposes. A stronger interaction with the target RNA may be achieved by increasing the length of the oligonucleotide, as well as the stretch of bases complementary to the micro RNA target sequence of a target RNA. On the other hand, the length may be decreased for better delivery and bioavailability. A reduced length will give a decreased tm value (melting temperature) of the oligonucleotide. However, increasing the concentration of the oligonucleotide may be used to counteract this. Also affinity increasing nucleotides and affinity increasing modifications may be used.

In a preferred embodiment, the length of the oligonucleotide is selected from the group consisting of: less than 60 bases, less than 50 bases, less than 40 bases, less than 39 bases, less than 38 bases, less than 37 bases, less than 36 bases, less than 35, less than 34 bases, less than 33 bases, less than 32 bases, less than 31 bases, bases, less than 30 bases, less than 29 bases, less than 28 bases, less than 27 bases, less than 26 bases, less than 25 bases, less than 24 bases, less than 23 bases, less than 22 bases, less than 21 bases, less than 20 bases, less than 19 bases, less than 18 bases, less than 17 bases, less than 16 bases, less than 15 bases, less than 14 bases, less than 13 bases, less than 12 bases, less than 11 bases, less than 10 bases, less than 9 bases, less than 8 bases, less than 7 bases, more than 60 bases, more than 50 bases, more than 40 bases, more than 39 bases, more than 38 bases, more than 37 more, more than 36 bases, more than 35, more than 34 bases, more than 33 bases, more than 32 bases, more than 31, more than 30 bases, more than 29 bases, more than 28 bases, more than 27 bases, more than 26 bases, more than 25 bases, more than 24 bases, more than 23 bases, more than 22 bases, more than 21 bases, more than 20 bases, more than 19 bases, more than 18 bases, more than 17 bases, more than 16 bases, more than 15 bases, more than 14 bases, more than 13 bases, more than 12 bases, more than 11 bases, more than 10 bases, more than 9 bases, more than 8 bases, more than 7 bases, more than 6 bases and more than 5 bases.

In another preferred embodiment, the length of the oligonucleotide is selected from the group consisting of between 10 and 14 bases, between 12 and 16 bases, between 14 and 18 bases, between 16 and 20, between 10 and 25 bases, between 12 and 24 bases, between 14 and 22 bases, between 15 and 22 bases and between 15 and 20 bases.

More preferred is a length between 8 and 25 bases.

Most preferred is a length between 10 and 20 bases.

In a preferred embodiment of the invention, the microRNA has a sequence selected from the group consisting of SEQ NO: 1-723.

Preferred microRNAs are also listed in table 2. Note that the sequences of the sequence list of the priority applications mentioned earlier have been renumbered and additional sequences have been added.

TABLE 2
A list of human micro RNAs.

| | | SEQ |
|---|---|---|
| | | ID |
| MicroRNA | Sequence | NO |
| hsa-let-7a | UGAGGUAGUAGGUUGUAUAGUU | 1 |
| hsa-let-7a* | CUAUACAAUCUACUGUCUUUC | 2 |
| hsa-let-7b | UGAGGUAGUAGGUUGUGUGGUU | 3 |
| hsa-let-7b* | CUAUACAACCUACUGCCUUCCC | 4 |
| hsa-let-7c | UGAGGUAGUAGGUUGUAUGGUU | 5 |
| hsa-let-7c* | UAGAGUUACACCCUGGGAGUUA | 6 |
| hsa-let-7d | AGAGGUAGUAGGUUGCAUAGUU | 7 |
| hsa-let-7d* | CUAUACGACCUGCUGCCUUUCU | 8 |
| hsa-let-7e | UGAGGUAGGAGGUUGUAUAGUU | 9 |
| hsa-let-7e* | CUAUACGGCCUCCUAGCUUUCC | 10 |
| hsa-let-7f | UGAGGUAGUAGAUUGUAUAGUU | 11 |
| hsa-let-7f-1* | CUAUACAAUCUAUUGCCUUCCC | 12 |
| hsa-let-7f-2* | CUAUACAGUCUACUGUCUUUCC | 13 |
| hsa-let-7g | UGAGGUAGUAGUUUGUACAGUU | 14 |
| hsa-let-7g* | CUGUACAGGCCACUGCCUUGC | 15 |
| hsa-let-7i | UGAGGUAGUAGUUUGUGCUGUU | 16 |
| hsa-let-7i* | CUGCGCAAGCUACUGCCUUGCU | 17 |
| hsa-miR-1 | UGGAAUGUAAAGAAGUAUGUAU | 18 |
| hsa-miR-100 | AACCCGUAGAUCCGAACUUGUG | 19 |
| hsa-miR-100* | CAAGCUUGUAUCUAUAGGUAUG | 20 |
| hsa-miR-101 | UACAGUACUGUGAUAACUGAA | 21 |
| hsa-miR-101* | CAGUUAUCACAGUGCUGAUGCU | 22 |
| hsa-miR-103 | AGCAGCAUUGUACAGGGCUAUGA | 23 |
| hsa-miR-105 | UCAAAUGCUCAGACUCCUGUGGU | 24 |
| hsa-miR-105* | ACGGAUGUUUGAGCAUGUGCUA | 25 |
| hsa-miR-106a | AAAAGUGCUUACAGUGCAGGUAG | 26 |
| hsa-miR-106a* | CUGCAAUGUAAGCACUUCUUAC | 27 |
| hsa-miR-106b | UAAAGUGCUGACAGUGCAGAU | 28 |
| hsa-miR-106b* | CCGCACUGUGGGUACUUGCUGC | 29 |
| hsa-miR-107 | AGCAGCAUUGUACAGGGCUAUCA | 30 |
| hsa-miR-10a | UACCCUGUAGAUCCGAAUUUGUG | 31 |
| hsa-miR-10a* | CAAAUUCGUAUCUAGGGGAAUA | 32 |
| hsa-miR-10b | UACCCUGUAGAACCGAAUUUGUG | 33 |
| hsa-miR-10b* | ACAGAUUCGAUUCUAGGGGAAU | 34 |
| hsa-miR-122 | UGGAGUGUGACAAUGGUGUUUG | 35 |
| hsa-miR-122* | AACGCCAUUAUCACACUAAAUA | 36 |
| hsa-miR-124 | UAAGGCACGCGGUGAAUGCC | 37 |
| hsa-miR-124* | CGUGUUCACAGCGGACCUUGAU | 38 |
| hsa-miR-125a-3p | ACAGGUGAGGUUCUUGGGAGCC | 39 |
| hsa-miR-125a-5p | UCCCUGAGACCCUUUAACCUGUGA | 40 |
| hsa-miR-125b | UCCCUGAGACCCUAACUUGUGA | 41 |
| hsa-miR-125b-1* | ACGGGUUAGGCUCUUGGGAGCU | 42 |
| hsa-miR-125b-2* | UCACAAGUCAGGCUCUUGGGAC | 43 |
| hsa-miR-126 | UCGUACCGUGAGUAAUAAUGCG | 44 |
| hsa-miR-126* | CAUUAUUACUUUUGGUACGCG | 45 |
| hsa-miR-127-3p | UCGGAUCCGUCUGAGCUUGGCU | 46 |
| hsa-miR-127-5p | CUGAAGCUCAGAGGGCUCUGAU | 47 |
| hsa-miR-128a | UCACAGUGAACCGGUCUCUUU | 48 |
| hsa-miR-128b | UCACAGUGAACCGGUCUCUUU | 49 |
| hsa-miR-129* | AAGCCCUUACCCCAAAAAGUAU | 50 |
| hsa-miR-129-3p | AAGCCCUUACCCCAAAAAGCAU | 51 |
| hsa-miR-129-5p | CUUUUUGCGGUCUGGGCUUGC | 52 |
| hsa-miR-130a | CAGUGCAAUGUUAAAAGGGCAU | 53 |
| hsa-miR-130a* | UUCACAUUGUGCUACUGUCUGC | 54 |
| hsa-miR-130b | CAGUGCAAUGAUGAAAGGGCAU | 55 |
| hsa-miR-130b* | ACUCUUUCCCUGUUGCACUAC | 56 |
| hsa-miR-132 | UAACAGUCUACAGCCAUGGUCG | 57 |
| hsa-miR-132* | ACCGUGGCUUUCGAUUGUUACU | 58 |
| hsa-miR-133a | UUUGGUCCCCUUCAACCAGCUG | 59 |
| hsa-miR-133b | UUUGGUCCCCUUCAACCAGCUA | 60 |
| hsa-miR-134 | UGUGACUGGUUGACCAGAGGGG | 61 |
| hsa-miR-135a | UAUGGCUUUUUAUUCCUAUGUGA | 62 |
| hsa-miR-135a* | UAUAGGGAUUGGAGCCGUGGCG | 63 |
| hsa-miR-135b | UAUGGCUUUUCAUUCCUAUGUGA | 64 |
| hsa-miR-135b* | AUGUAGGGCUAAAAGCCAUGGG | 65 |
| hsa-miR-136 | ACUCCAUUUGUUUUGAUGAUGGA | 66 |
| hsa-miR-136* | CAUCAUCGUCUCAAAUGAGUCU | 67 |
| hsa-miR-137 | UUAUUGCUUAAGAAUACGCGUAG | 68 |
| hsa-miR-138 | AGCUGGUGUUGUGAAUCAGGCCG | 69 |
| hsa-miR-138-1* | GCUACUUCACAACACCAGGGCC | 70 |
| hsa-miR-138-2* | GCUAUUUCACGACACCAGGGUU | 71 |
| hsa-miR-139-3p | GGAGACGCGGCCCUGUUGGAGU | 72 |
| hsa-miR-139-5p | UCUACAGUGCACGUGUCUCCAG | 73 |
| hsa-miR-140-3p | UACCACAGGGUAGAACCACGG | 74 |
| hsa-miR-140-5p | CAGUGGUUUUACCCUAUGGUAG | 75 |
| hsa-miR-141 | UAACACUGUCUGGUAAAGAUGG | 76 |
| hsa-miR-141* | CAUCUUCCAGUACAGUGUUGGA | 77 |
| hsa-miR-142-3p | UGUAGUGUUUCCUACUUUAUGGA | 78 |
| hsa-miR-142-5p | CAUAAAGUAGAAAGCACUACU | 79 |
| hsa-miR-143 | UGAGAUGAAGCACUGUAGCUC | 80 |
| hsa-miR-143* | GGUGCAGUGCUGCAUCUCUGGU | 81 |
| hsa-miR-144 | UACAGUAUAGAUGAUGUACU | 82 |
| hsa-miR-144* | GGAUAUCAUCAUAUACUGUAAG | 83 |
| hsa-miR-145 | GUCCAGUUUUCCCAGGAAUCCCU | 84 |
| hsa-miR-145* | GGAUUCCUGGAAAUACUGUUCU | 85 |
| hsa-miR-146a | UGAGAACUGAAUUCCAUGGGUU | 86 |
| hsa-miR-146a* | CCUCUGAAAUUCAGUUCUUCAG | 87 |
| hsa-miR-146b-3p | UGCCCUGUGGACUCAGUUCUGG | 88 |
| hsa-miR-146b-5p | UGAGAACUGAAUUCCAUAGGCU | 89 |
| hsa-miR-147 | GUGUGUGGAAAUGCUUCUGC | 90 |
| hsa-miR-147b | GUGUGCGGAAAUGCUUCUGCUA | 91 |
| hsa-miR-148a | UCAGUGCACUACAGAACUUUGU | 92 |
| hsa-miR-148a* | AAAGUUCUGAGACACUCCGACU | 93 |
| hsa-miR-148b | UCAGUGCAUCACAGAACUUUGU | 94 |
| hsa-miR-148b* | AAGUUCUGUUAUACACUCAGGC | 95 |
| hsa-miR-149 | UCUGGCUCCGUGUCUUCACUCCC | 96 |
| hsa-miR-149* | AGGGAGGGACGGGGGCUGUGC | 97 |
| hsa-miR-150 | UCUCCCAACCCUUGUACCAGUG | 98 |
| hsa-miR-150* | CUGGUACAGGCCUGGGGGACAG | 99 |
| hsa-miR-151-3p | CUAGACUGAAGCUCCUUGAGG | 100 |
| hsa-miR-151-5p | UCGAGGAGCUCACAGUCUAGU | 101 |
| hsa-miR-152 | UCAGUGCAUGACAGAACUUGG | 102 |
| hsa-miR-153 | UUGCAUAGUCACAAAAGUGAUC | 103 |
| hsa-miR-154 | UAGGUUAUCCGUGUUGCCUUCG | 104 |
| hsa-miR-154* | AAUCAUACACGGUUGACCUAUU | 105 |
| hsa-miR-155 | UUAAUGCUAAUCGUGAUAGGGGU | 106 |
| hsa-miR-155* | CUCCUACAUAUUAGCAUUAACA | 107 |
| hsa-miR-15a | UAGCAGCACAUAAUGGUUUGUG | 108 |
| hsa-miR-15a* | CAGGCCAUAUUGUGCUGCCUCA | 109 |
| hsa-miR-15b | UAGCAGCACAUCAUGGUUUACA | 110 |
| hsa-miR-15b* | CGAAUCAUUAUUUGCUGCUCUA | 111 |
| hsa-miR-16 | UAGCAGCACGUAAAUAUUGGCG | 112 |
| hsa-miR-16-1* | CCAGUAUUAACUGUGCUGCUGA | 113 |
| hsa-miR-16-2* | CCAAUAUUACUGUGCUGCUUUA | 114 |
| hsa-miR-17 | CAAAGUGCUUACAGUGCAGGUAG | 115 |
| hsa-miR-17* | ACUGCAGUGAAGGCACUUGUAG | 116 |
| hsa-miR-181a | AACAUUCAACGCUGUCGGUGAGU | 117 |
| hsa-miR-181a* | ACCAUCGACCGUUGAUUGUACC | 118 |
| hsa-miR-181a-2* | ACCACUGACCGUUGACUGUACC | 119 |
| hsa-miR-181b | AACAUUCAUUGCUGUCGGUGGGU | 120 |
| hsa-miR-181c | AACAUUCAACCUGUCGGUGAGU | 121 |
| hsa-miR-181c* | AACCAUCGACCGUUGAGUGGAC | 122 |
| hsa-miR-181d | AACAUUCAUUGUUGUCGGUGGGU | 123 |
| hsa-miR-182 | UUUGGCAAUGGUAGAACUCACACU | 124 |
| hsa-miR-182* | UGGUUCUAGACUUGCCAACUA | 125 |
| hsa-miR-183 | UAUGGCACUGGUAGAAUUCACU | 126 |
| hsa-miR-183* | GUGAAUUACCGAAGGGCCAUAA | 127 |
| hsa-miR-184 | UGGACGGAGAACUGAUAAGGGU | 128 |
| hsa-miR-185 | UGGAGAGAAAGGCAGUUCCUGA | 129 |
| hsa-miR-185* | AGGGGCUGGCUUUCCUCUGGUC | 130 |
| hsa-miR-186 | CAAAGAAUUCUCCUUUUGGGCU | 131 |
| hsa-miR-186* | GCCCAAAGGUGAAUUUUUUGGG | 132 |
| hsa-miR-187 | UCGUGUCUUGUGUUGCAGCCGG | 133 |
| hsa-miR-187* | GGCUACAACACAGGACCCGGGC | 134 |
| hsa-miR-188-3p | CUCCCACAUGCAGGGUUUGCA | 135 |
| hsa-miR-188-5p | CAUCCCUUGCAUGGUGGAGGG | 136 |
| hsa-miR-18a | UAAGGUGCAUCUAGUGCAGAUAG | 137 |
| hsa-miR-18a* | ACUGCCCUAAGUGCUCCUUCUGG | 138 |
| hsa-miR-18b | UAAGGUGCAUCUAGUGCAGUUAG | 139 |
| hsa-miR-18b* | UGCCCUAAAUGCCCCUUCUGGC | 140 |
| hsa-miR-190 | UGAUAUGUUUGAUAUAUUAGGU | 141 |
| hsa-miR-190b | UGAUAUGUUUGAUAUUGGGUU | 142 |
| hsa-miR-191 | CAACGGAAUCCCAAAAGCAGCUG | 143 |
| hsa-miR-191* | GCUGCGCUUGGAUUUCGUCCCC | 144 |
| hsa-miR-192 | CUGACCUAUGAAUUGACAGCC | 145 |
| hsa-miR-192* | CUGCCAAUUCCAUAGGUCACAG | 146 |
| hsa-miR-193a-3p | AACUGGCCUACAAAGUCCCAGU | 147 |
| hsa-miR-193a-5p | UGGGUCUUUGCGGGCGAGAUGA | 148 |
| hsa-miR-193b | AACUGGCCCUCAAAGUCCCGCU | 149 |
| hsa-miR-193b* | CGGGGUUUUGAGGGCGAGAUGA | 150 |
| hsa-miR-194 | UGUAACAGCAACUCCAUGUGGA | 151 |
| hsa-miR-194* | CCAGUGGGGCUGCUGUUAUCUG | 152 |
| hsa-miR-195 | UAGCAGCACAGAAAUAUUGGC | 153 |
| hsa-miR-195* | CCAAUAUUGGCUGUGCUGCUCC | 154 |
| hsa-miR-196a | UAGGUAGUUUCAUGUUGUUGGG | 155 |
| hsa-miR-196a* | CGGCAACAAGAAACUGCCUGAG | 156 |
| hsa-miR-196b | UAGGUAGUUUCCUGUUGUUGGG | 157 |
| hsa-miR-197 | UUCACCACCUUCUCCACCCAGC | 158 |
| hsa-miR-198 | GGUCCAGAGGGGAGAUAGGUUC | 159 |
| hsa-miR-199a-3p | ACAGUAGUCUGCACAUUGGUUA | 160 |
| hsa-miR-199a-5p | CCCAGUGUUCAGACUACCUGUUC | 161 |
| hsa-miR-199b-3p | ACAGUAGUCUGCACAUUGGUUA | 162 |
| hsa-miR-199b-5p | CCCAGUGUUUAGACUAUCUGUUC | 163 |
| hsa-miR-19a | UGUGCAAAUCUAUGCAAAACUGA | 164 |
| hsa-miR-19a* | AGUUUUGCAUAGUUGCACUACA | 165 |
| hsa-miR-19b | UGUGCAAAUCCAUGCAAAACUGA | 166 |
| hsa-miR-19b-1* | AGUUUUGCAGGUUUGCAUCCAGC | 167 |
| hsa-miR-19b-2* | AGUUUUGCAGGUUUGCAUUUCA | 168 |
| hsa-miR-200a | UAACACUGUCUGGUAACGAUGU | 169 |
| hsa-miR-200a* | CAUCUUACCGGACAGUGCUGGA | 170 |
| hsa-miR-200b | UAAUACUGCCUGGUAAUGAUGA | 171 |
| hsa-miR-200b* | CAUCUUACUGGGCAGCAUUGGA | 172 |
| hsa-miR-200c | UAAUACUGCCGGGUAAUGAUGGA | 173 |
| hsa-miR-200c* | CGUCUUACCCAGCAGUGUUUGG | 174 |
| hsa-miR-202 | AGAGGUAUAGGGCAUGGGAA | 175 |
| hsa-miR-202* | UUCCUAUGCAUAUACUUCUUUG | 176 |
| hsa-miR-203 | GUGAAAUGUUUAGGACCACUAG | 177 |
| hsa-miR-204 | UUCCCUUUGUCAUCCUAUGCCU | 178 |
| hsa-miR-205 | UCCUUCAUUCCACCGGAGUCUG | 179 |
| hsa-miR-206 | UGGAAUGUAAGGAAGUGUGUGG | 180 |
| hsa-miR-208 | AUAAGACGAGCAAAAAGCUUGU | 181 |
| hsa-miR-208b | AUAAGACGAACAAAAGGUUUGU | 182 |
| hsa-miR-20a | UAAAGUGCUUAUAGUGCAGGUAG | 183 |
| hsa-miR-20a* | ACUGCAUUAUGAGCACUUAAAG | 184 |
| hsa-miR-20b | CAAAGUGCUCAUAGUGCAGGUAG | 185 |
| hsa-miR-20b* | ACUGUAGUAUGGGCACUUCCAG | 186 |
| hsa-miR-21 | UAGCUUAUCAGACUGAUGUUGA | 187 |
| hsa-miR-21* | CAACACCAGUCGAUGGGCUGU | 188 |
| hsa-miR-210 | CUGUGCGUGUGACAGCGGCUGA | 189 |
| hsa-miR-211 | UUCCCUUUGUCAUCCUUCGCCU | 190 |
| hsa-miR-212 | UAACAGUCUCCAGUCACGGCC | 191 |
| hsa-miR-214 | ACAGCAGGCACAGACAGGCAGU | 192 |
| hsa-miR-214* | UGCCUGUCUACACUUGCUGUGC | 193 |
| hsa-miR-215 | AUGACCUAUGAAUUGACAGAC | 194 |
| hsa-miR-216a | UAAUCUCAGCUGGCAACUGUGA | 195 |
| hsa-miR-216b | AAAUCUCUGCAGGCAAAUGUGA | 196 |
| hsa-miR-217 | UACUGCAUCAGGAACUGAUUGGA | 197 |
| hsa-miR-218 | UUGUGCUUGAUCUAACCAUGU | 198 |
| hsa-miR-218-1* | AUGGUUCCGUCAAGCACCAUGG | 199 |
| hsa-miR-218-2* | CAUGGUUCUGUCAAGCACCGCG | 200 |
| hsa-miR-219-1-3p | AGAGUUGAGUCUGGACGUCCCG | 201 |
| hsa-miR-219-2-3p | AGAAUUGUGGCUGGACAUCUGU | 202 |
| hsa-miR-219-5p | UGAUUGUCCAAACGCAAUUCU | 203 |
| hsa-miR-22 | AAGCUGCCAGUUGAAGAACUGU | 204 |
| hsa-miR-22* | AGUUCUUCAGUGGCAAGCUUUA | 205 |
| hsa-miR-220 | CCACACCGUAUCUGACACUUU | 206 |
| hsa-miR-220b | CCACCACCGUGUCUGACACUU | 207 |
| hsa-miR-220c | ACACAGGGCUGUUGUGAAGACU | 208 |
| hsa-miR-221 | AGCUACAUUGUCUGCUGGGUUUC | 209 |
| hsa-miR-221* | ACCUGGCAUACAAUGUAGAUUU | 210 |
| hsa-miR-222 | AGCUACAUCUGGCUACUGGGU | 211 |
| hsa-miR-222* | CUCAGUAGCCAGUGUAGAUCCU | 212 |
| hsa-miR-223 | UGUCAGUUUGUCAAAUACCCCA | 213 |
| hsa-miR-223* | CGUGUAUUUGACAAGCUGAGUU | 214 |
| hsa-miR-224 | CAAGUCACUAGUGGUUCCGUU | 215 |
| hsa-miR-23a | AUCACAUUGCCAGGGAUUUCC | 216 |
| hsa-miR-23a* | GGGGUUCCUGGGGAUGGGAUUU | 217 |
| hsa-miR-23b | AUCACAUUGCCAGGGAUUACC | 218 |
| hsa-miR-23b* | UGGGUUCCUGGCAUGCUGAUUU | 219 |
| hsa-miR-24 | UGGCUCAGUUCAGCAGGAACAG | 220 |
| hsa-miR-24-1* | UGCCUACUGAGCUGAUAUCAGU | 221 |
| hsa-miR-24-2* | UGCCUACUGAGCUGAAACACAG | 222 |
| hsa-miR-25 | CAUUGCACUUGUCUCGGUCUGA | 223 |
| hsa-miR-25* | AGGCGGAGACUUGGGCAAUUG | 224 |
| hsa-miR-26a | UUCAAGUAAUCCAGGAUAGGCU | 225 |
| hsa-miR-26a-1* | CCUAUUCUUGGUUACUUGCACG | 226 |
| hsa-miR-26a-2* | CCUAUUCUUGAUUACUUGUUUC | 227 |
| hsa-miR-26b | UUCAAGUAAUUCAGGAUAGGU | 228 |
| hsa-miR-26b* | CCUGUUCUCCAUUACUUGGCUC | 229 |
| hsa-miR-27a | UUCACAGUGGCUAAGUUCCGC | 230 |
| hsa-miR-27a* | AGGGCUUAGCUGCUUGUGAGCA | 231 |
| hsa-miR-27b | UUCACAGUGGCUAAGUUCUGC | 232 |
| hsa-miR-27b* | AGAGCUUAGCUGAUUGGUGAAC | 233 |
| hsa-miR-28-3p | CACUAGAUUGUGAGCUCCUGGA | 234 |
| hsa-miR-28-5p | AAGGAGCUCACAGUCUAUUGAG | 235 |
| hsa-miR-296-3p | GAGGGUUGGGUGGAGGCUCUCC | 236 |
| hsa-miR-296-5p | AGGGCCCCCCCUCAAUCCUGU | 237 |
| hsa-miR-297 | AUGUAUGUGUGCAUGUGCAUG | 238 |
| hsa-miR-298 | AGCAGAAGCAGGGAGGUUCUCCCA | 239 |
| hsa-miR-299-3p | UAUGUGGGAUGGUAAACCGCUU | 240 |
| hsa-miR-299-5p | UGGUUUACCGUCCCACAUACAU | 241 |
| hsa-miR-29a | UAGCACCAUCUGAAAUCGGUUA | 242 |
| hsa-miR-29a* | ACUGAUUUCUUUUGGUGUUCAG | 243 |
| hsa-miR-29b | UAGCACCAUUUGAAAUCAGUGUU | 244 |
| hsa-miR-29b-1* | GCUGGUUUCAUAUGGUGGUUUAGA | 245 |
| hsa-miR-29b-2* | CUGGUUUCACAUGGUGGCUUAG | 246 |
| hsa-miR-29c | UAGCACCAUUUGAAAUCGGUUA | 247 |
| hsa-miR-29c* | UGACCGAUUUCUCCUGGUGUUC | 248 |
| hsa-miR-300 | UAUACAAGGGCAGACUCUCUCU | 249 |
| hsa-miR-301a | CAGUGCAAUAGUAUUGUCAAAGC | 250 |
| hsa-miR-301b | CAGUGCAAUGAUAUUGUCAAAGC | 251 |
| hsa-miR-302a | UAAGUGCUUCCAUGUUUUGGUGA | 252 |
| hsa-miR-302a* | ACUUAAACGUGGAUGUACUUGCU | 253 |
| hsa-miR-302b | UAAGUGCUUCCAUGUUUUAGUAG | 254 |
| hsa-miR-302b* | ACUUUAACAUGGAAGUGCUUUC | 255 |
| hsa-miR-302c | UAAGUGCUUCCAUGUUUCAGUGG | 256 |
| hsa-miR-302c* | UUUAACAUGGGGGUACCUGCUG | 257 |
| hsa-miR-302d | UAAGUGCUUCCAUGUUUGAGUGU | 258 |
| hsa-miR-302d* | ACUUUAACAUGGAGGCACUUGC | 259 |
| hsa-miR-30a | UGUAAACAUCCUCGACUGGAAG | 260 |
| hsa-miR-30a* | CUUUCAGUCGGAUGUUUGCAGC | 261 |
| hsa-miR-30b | UGUAAACAUCCUACACUCAGCU | 262 |
| hsa-miR-30b* | CUGGGAGGUGGAUGUUUACUUC | 263 |
| hsa-miR-30c | UGUAAACAUCCUACACUCUCAGC | 264 |
| hsa-miR-30c-1* | CUGGGAGAGGGUUGUUUACUCC | 265 |
| hsa-miR-30c-2* | CUGGGAGAAGGCUGUUUACUCU | 266 |
| hsa-miR-30d | UGUAAACAUCCCCGACUGGAAG | 267 |
| hsa-miR-30d* | CUUUCAGUCAGAUGUUUGCUGC | 268 |
| hsa-miR-30e | UGUAAACAUCCUUGACUGGAAG | 269 |
| hsa-miR-30e* | CUUUCAGUCGGAUGUUUACAGC | 270 |
| hsa-miR-31 | AGGCAAGAUGCUGGCAUAGCU | 271 |
| hsa-miR-31* | UGCUAUGCCAACAUAUUGCCAU | 272 |
| hsa-miR-32 | UAUUGCACAUUACUAAGUUGCA | 273 |
| hsa-miR-32* | CAAUUUAGUGUGUGUGAUAUUU | 274 |
| hsa-miR-320 | AAAAGCUGGGUUGAGAGGGCGA | 275 |
| hsa-miR-323-3p | CACAUUACACGGUCGACCUCU | 276 |
| hsa-miR-323-5p | AGGUGGUCCGUGGCGCGUUCGC | 277 |
| hsa-miR-324-3p | ACUGCCCCAGGUGCUGCUGG | 278 |
| hsa-miR-324-5p | CGCAUCCCCUAGGGCAUUGGUGU | 279 |
| hsa-miR-325 | CCUAGUAGGUGUCCAGUAAGUGU | 280 |
| hsa-miR-326 | CCUCUGGGCCCUUCCUCCAG | 281 |
| hsa-miR-328 | CUGGCCCUCUCUGCCCUUCCGU | 282 |
| hsa-miR-329 | AACACACCUGGUUAACCUCUUU | 283 |
| hsa-miR-330-3p | GCAAAGCACACGGCCUGCAGAGA | 284 |
| hsa-miR-330-5p | UCUCUGGGCCUGUGUCUUAGGC | 285 |
| hsa-miR-331-3p | GCCCCUGGGCCUAUCCUAGAA | 286 |
| hsa-miR-331-5p | CUAGGUAUGGUCCCAGGGAUCC | 287 |
| hsa-miR-335 | UCAAGAGCAAUAACGAAAAAUGU | 288 |
| hsa-miR-335* | UUUUUCAUUAUUGCUCCUGACC | 289 |
| hsa-miR-337-3p | CUCCUAUAUGAUGCCUUUCUUC | 290 |
| hsa-miR-337-5p | GAACGGCUUCAUACAGGAGUU | 291 |
| hsa-miR-338-3p | UCCAGCAUCAGUGAUUUUGUUG | 292 |
| hsa-miR-338-5p | AACAAUAUCCUGGUGCUGAGUG | 293 |
| hsa-miR-339-3p | UGAGCGCCUCGACGACAGAGCCG | 294 |
| hsa-miR-339-5p | UCCCUGUCCUCCAGGAGCUCACG | 295 |
| hsa-miR-33a | GUGCAUUGUAGUUGCAUUGCA | 296 |
| hsa-miR-33a* | CAAUGUUUCCACAGUGCAUCAC | 297 |
| hsa-miR-33b | GUGCAUUGCUGUUGCAUUGC | 298 |
| hsa-miR-33b* | CAGUGCCUCGGCAGUGCAGCCC | 299 |
| hsa-miR-340 | UUAUAAAGCAAUGAGACUGAUU | 300 |
| hsa-miR-340* | UCCGUCUCAGUUACUUUAUAGC | 301 |
| hsa-miR-342-3p | UCUCACACAGAAAUCGCACCCGU | 302 |
| hsa-miR-342-5p | AGGGGUGCUAUCUGUGAUUGA | 303 |
| hsa-miR-345 | GCUGACUCCUAGUCCAGGGCUC | 304 |
| hsa-miR-346 | UGUCUGCCCGCAUGCCUGCCUCU | 305 |
| hsa-miR-34a | UGGCAGUGUCUUAGCUGGUUGU | 306 |
| hsa-miR-34a* | CAAUCAGCAAGUAUACUGCCCU | 307 |
| hsa-miR-34b | CAAUCACUAACUCCACUGCCAU | 308 |
| hsa-miR-34b* | UAGGCAGUGUCAUUAGCUGAUUG | 309 |
| hsa-miR-34c-3p | AAUCACUAACCACACGGCCAGG | 310 |
| hsa-miR-34c-5p | AGGCAGUGUAGUUAGCUGAUUGC | 311 |
| hsa-miR-361-3p | UCCCCCAGGUGUGAUUCUGAUUU | 312 |
| hsa-miR-361-5p | UUAUCAGAAUCUCCAGGGGUAC | 313 |
| hsa-miR-362-3p | AACACACCUAUUCAAGGAUUCA | 314 |
| hsa-miR-362-5p | AAUCCUUGGAACCUAGGUGUGAGU | 315 |
| hsa-miR-363 | AAUUGCACGGUAUCCAUCUGUA | 316 |
| hsa-miR-363* | CGGGUGGAUCACGAUGCAAUUU | 317 |
| hsa-miR-365 | UAAUGCCCCUAAAAAUCCUUAU | 318 |
| hsa-miR-367 | AAUUGCACUUUAGCAAUGGUGA | 319 |
| hsa-miR-367* | ACUGUUGCUAAUAUGCAACUCU | 320 |
| hsa-miR-369-3p | AAUAAUACAUGGUUGAUCUUU | 321 |
| hsa-miR-369-5p | AGAUCGACCGUGUUAUAUUCGC | 322 |
| hsa-miR-370 | GCCUGCUGGGGUGGAACCUGGU | 323 |
| hsa-miR-371-3p | AAGUGCCGCCAUCUUUUGAGUGU | 324 |
| hsa-miR-371-5p | ACUCAAACUGUGGGGGCACU | 325 |
| hsa-miR-372 | AAAGUGCUGCGACAUUUGAGCGU | 326 |
| hsa-miR-373 | GAAGUGCUUCGAUUUUGGGGUGU | 327 |
| hsa-miR-373* | ACUCAAAAUGGGGGCGCUUUCC | 328 |
| hsa-miR-374a | UUAUAAUACAACCUGAUAAGUG | 329 |
| hsa-miR-374a* | CUUAUCAGAUUGUAUUGUAAUU | 330 |
| hsa-miR-374b | AUAUAAUACAACCUGCUAAGUG | 331 |
| hsa-miR-374b* | CUUAGCAGGUUGUAUUAUCAUU | 332 |
| hsa-miR-375 | UUUGUUCGUUCGGCUCGCGUGA | 333 |
| hsa-miR-376a | AUCAUAGAGGAAAAUCCACGU | 334 |
| hsa-miR-376a* | GUAGAUUCUCCUUCUAUGAGUA | 335 |
| hsa-miR-376b | AUCAUAGAGGAAAAUCCAUGUU | 336 |
| hsa-miR-376c | AACAUAGAGGAAAUUCCACGU | 337 |
| hsa-miR-377 | AUCACACAAAGGCAACUUUUGU | 338 |
| hsa-miR-377* | AGAGGUUGCCCUUGGUGAAUUC | 339 |
| hsa-miR-378 | ACUGGACUUGGAGUCAGAAGG | 340 |
| hsa-miR-378* | CUCCUGACUCCAGGUCCUGUGU | 341 |
| hsa-miR-379 | UGGUAGACUAUGGAACGUAGG | 342 |
| hsa-miR-379* | UAUGUAACAUGGUCCACUAACU | 343 |
| hsa-miR-380 | UAUGUAAUAUGGUCCACAUCUU | 344 |
| hsa-miR-380* | UGGUUGACCAUAGAACAUGCGC | 345 |
| hsa-miR-381 | UAUACAAGGGCAAGCUCUCUGU | 346 |
| hsa-miR-382 | GAAGUUGUUCGUGGUGGAUUCG | 347 |
| hsa-miR-383 | AGAUCAGAAGGUGAUUGUGGCU | 348 |
| hsa-miR-384 | AUUCCUAGAAAUUGUUCAUA | 349 |
| hsa-miR-409-3p | GAAUGUUGCUCGGUGAACCCCU | 350 |
| hsa-miR-409-5p | AGGUUACCCGAGCAACUUUGCAU | 351 |
| hsa-miR-410 | AAUAUAACACAGAUGGCCUGU | 352 |
| hsa-miR-411 | UAGUAGACCGUAUAGCGUACG | 353 |
| hsa-miR-411* | UAUGUAACACGGUCCACUAACC | 354 |
| hsa-miR-412 | ACUUCACCUGGUCCACUAGCCGU | 355 |
| hsa-miR-421 | AUCAACAGACAUUAAUUGGGCGC | 356 |
| hsa-miR-422a | ACUGGACUUAGGGUCAGAAGGC | 357 |
| hsa-miR-423-3p | AGCUCGGUCUGAGGCCCCUCAGU | 358 |
| hsa-miR-423-5p | UGAGGGGCAGAGAGCGAGACUUU | 359 |
| hsa-miR-424 | CAGCAGCAAUUCAUGUUUUGAA | 360 |
| hsa-miR-424* | CAAAACGUGAGGCGCUGCUAU | 361 |
| hsa-miR-425 | AAUGACACGAUCACUCCCGUUGA | 362 |
| hsa-miR-425* | AUCGGGAAUGUCGUGUCCGCCC | 363 |
| hsa-miR-429 | UAAUACUGUCUGGUAAAACCGU | 364 |
| hsa-miR-431 | UGUCUUGCAGGCCGUCAUGCA | 365 |
| hsa-miR-431* | CAGGUCGUCUUGCAGGGCUUCU | 366 |
| hsa-miR-432 | UCUUGGAGUAGGUCAUUGGGUGG | 367 |
| hsa-miR-432* | CUGGAUGGCUCCUCCAUGUCU | 368 |
| hsa-miR-433 | AUCAUGAUGGGCUCCUCGGUGU | 369 |
| hsa-miR-448 | UUGCAUAUGUAGGAUGUCCCAU | 370 |
| hsa-miR-449a | UGGCAGUGUAUUGUUAGCUGGU | 371 |
| hsa-miR-449b | AGGCAGUGUAUUGUUAGCUGGC | 372 |
| hsa-miR-450a | UUUUGCGAUGUGUUCCUAAUAU | 373 |
| hsa-miR-450b-3p | UUGGGAUCAUUUUGCAUCCAUA | 374 |
| hsa-miR-450b-5p | UUUUGCAAUAUGUUCCUGAAUA | 375 |
| hsa-miR-451 | AAACCGUUACCAUUACUGAGUU | 376 |
| hsa-miR-452 | AACUGUUUGCAGAGGAAACUGA | 377 |
| hsa-miR-452* | CUCAUCUGCAAAGAAGUAAGUG | 378 |
| hsa-miR-453 | AGGUUGUCCGUGGUGAGUUCGCA | 379 |
| hsa-miR-454 | UAGUGCAAUAUUGCUUAUAGGGU | 380 |
| hsa-miR-454* | ACCCUAUCAAUAUUGUCUCUGC | 381 |
| hsa-miR-455-3p | GCAGUCCAUGGGCAUAUACAC | 382 |
| hsa-miR-455-5p | UAUGUGCCUUUGGACUACAUCG | 383 |
| hsa-miR-483-3p | UCACUCCUCUCCUCCCGUCUU | 384 |
| hsa-miR-483-5p | AAGACGGGAGGAAAGAAGGGAG | 385 |
| hsa-miR-484 | UCAGGCUCAGUCCCCUCCCGAU | 386 |
| hsa-miR-485-3p | GUCAUACACGGCUCUCCUCUCU | 387 |
| hsa-miR-485-5p | AGAGGCUGGCCGUGAUGAAUUC | 388 |
| hsa-miR-486-3p | CGGGGCAGCUCAGUACAGGAU | 389 |
| hsa-miR-486-5p | UCCUGUACUGAGCUGCCCCGAG | 390 |
| hsa-miR-487a | AAUCAUACAGGGACAUCCAGUU | 391 |
| hsa-miR-487b | AAUCGUACAGGGUCAUCCACUU | 392 |
| hsa-miR-488 | UUGAAAGGCUAUUUCUUGGUC | 393 |
| hsa-miR-488* | CCCAGAUAAUGGCACUCUCAA | 394 |
| hsa-miR-489 | GUGACAUCACAUAUACGGCAGC | 395 |
| hsa-miR-490-3p | CAACCUGGAGGACUCCAUGCUG | 396 |
| hsa-miR-490-5p | CCAUGGAUCUCCAGGUGGGU | 397 |
| hsa-miR-491-3p | CUUAUGCAAGAUUCCCUUCUAC | 398 |
| hsa-miR-491-5p | AGUGGGGAACCCUUCCAUGAGG | 399 |
| hsa-miR-492 | AGGACCUGCGGGACAAGAUUCUU | 400 |
| hsa-miR-493 | UGAAGGUCUACUGUGUGCCAGG | 401 |
| hsa-miR-493* | UUGUACAUGGUAGGCUUUCAUU | 402 |
| hsa-miR-494 | UGAAACAUACACGGGAAACCUC | 403 |
| hsa-miR-495 | AAACAAACAUGGUGCACUUCUU | 404 |
| hsa-miR-496 | UGAGUAUUACAUGGCCAAUCUC | 405 |
| hsa-miR-497 | CAGCAGCACACUGUGGUUUGU | 406 |
| hsa-miR-497* | CAAACCACACUGUGGUGUUAGA | 407 |
| hsa-miR-498 | UUUCAAGCCAGGGGGCGUUUUUC | 408 |
| hsa-miR-499-3p | AACAUCACAGCAAGUCUGUGCU | 409 |
| hsa-miR-499-5p | UUAAGACUUGCAGUGAUGUUU | 410 |
| hsa-miR-500 | UAAUCCUUGCUACCUGGGUGAGA | 411 |
| hsa-miR-500* | AUGCACCUGGGCAAGGAUUCUG | 412 |
| hsa-miR-501-3p | AAUGCACCCGGGCAAGGAUUCU | 413 |
| hsa-miR-501-5p | AAUCCUUUGUCCCUGGGUGAGA | 414 |
| hsa-miR-502-3p | AAUGCACCUGGGCAAGGAUUCA | 415 |
| hsa-miR-502-5p | AUCCUUGCUAUCUGGGUGCUA | 416 |
| hsa-miR-503 | UAGCAGCGGGAACAGUUCUGCAG | 417 |
| hsa-miR-504 | AGACCCUGGUCUGCACUCUAUC | 418 |
| hsa-miR-505 | CGUCAACACUUGCUGGUUUCCU | 419 |
| hsa-miR-505* | GGGAGCCAGGAAGUAUUGAUGU | 420 |
| hsa-miR-506 | UAAGGCACCCUUCUGAGUAGA | 421 |
| hsa-miR-507 | UUUUGCACCUUUUGGAGUGAA | 422 |
| hsa-miR-508-3p | UGAUUGUAGCCUUUUGGAGUAGA | 423 |
| hsa-miR-508-5p | UACUCCAGAGGGCGUCACUCAUG | 424 |
| hsa-miR-509-3-5p | UACUGCAGACGUGGCAAUCAUG | 425 |
| hsa-miR-509-3p | UGAUUGGUACGUCUGUGGGUAG | 426 |
| hsa-miR-509-5p | UACUGCAGACAGUGGCAAUCA | 427 |
| hsa-miR-510 | UACUCAGGAGAGUGGCAAUCAC | 428 |
| hsa-miR-511 | GUGUCUUUUGCUCUGCAGUCA | 429 |
| hsa-miR-512-3p | AAGUGCUGUCAUAGCUGAGGUC | 430 |
| hsa-miR-512-5p | CACUCAGCCUUGAGGGCACUUUC | 431 |
| hsa-miR-513-3p | UAAAUUUCACCUUUCUGAGAAGG | 432 |
| hsa-miR-513-5p | UUCACAGGGAGGUGUCAU | 433 |
| hsa-miR-514 | AUUGACACUUCUGUGAGUAGA | 434 |
| hsa-miR-515-3p | GAGUGCCUUCUUUUGGAGCGUU | 435 |
| hsa-miR-515-5p | UUCUCCAAAAGAAAGCACUUUCUG | 436 |
| hsa-miR-516a-3p | UGCUUCCUUUCAGAGGGU | 437 |
| hsa-miR-516a-5p | UUCUCGAGGAAAGAAGCACUUUC | 438 |
| hsa-miR-516b | AUCUGGAGGUAAGAAGCACUUU | 439 |
| hsa-miR-516b* | UGCUUCCUUUCAGAGGGU | 440 |
| hsa-miR-517* | CCUCUAGAUGGAAGCACUGUCU | 441 |
| hsa-miR-517a | AUCGUGCAUCCCUUUAGAGUGU | 442 |
| hsa-miR-517b | UCGUGCAUCCCUUUAGAGUGUU | 443 |
| hsa-miR-517c | AUCGUGCAUCCUUUUAGAGUGU | 444 |
| hsa-miR-518a-3p | GAAAGCGCUUCCCUUUGCUGGA | 445 |
| hsa-miR-518a-5p | CUGCAAAGGGAAGCCCUUUC | 446 |
| hsa-miR-518b | CAAAGCGCUCCCCUUUAGAGGU | 447 |
| hsa-miR-518c | CAAAGCGCUUCUCUUUAGAGUGU | 448 |
| hsa-miR-518c* | UCUCUGGAGGGAAGCACUUUCUG | 449 |
| hsa-miR-518d-3p | CAAAGCGCUUCCCUUUGGAGC | 450 |
| hsa-miR-518d-5p | CUCUAGAGGGAAGCACUUUCUG | 451 |
| hsa-miR-518e | AAAGCGCUUCCCUUCAGAGUG | 452 |
| hsa-miR-518e* | CUCUAGAGGGAAGCGCUUUCUG | 453 |
| hsa-miR-518f | GAAAGCGCUUCUCUUUAGAGG | 454 |
| hsa-miR-518f* | CUCUAGAGGGAAGCACUUUCUC | 455 |
| hsa-miR-519a | AAAGUGCAUCCUUUUAGAGUGU | 456 |
| hsa-miR-519a* | CUCUAGAGGGAAGCGCUUUCUG | 457 |
| hsa-miR-519b-3p | AAAGUGCAUCCUUUUAGAGGUU | 458 |
| hsa-miR-519b-5p | CUCUAGAGGGAAGCGCUUUCUG | 459 |
| hsa-miR-519c-3p | AAAGUGCAUCUUUUUAGAGGAU | 460 |
| hsa-miR-519c-5p | CUCUAGAGGGAAGCGCUUUCUG | 461 |
| hsa-miR-519d | CAAAGUGCCUCCCUUUAGAGUG | 462 |
| hsa-miR-519e | AAGUGCCUCCUUUUAGAGUGUU | 463 |
| hsa-miR-519e* | UUCUCCAAAAGGGAGCACUUUC | 464 |
| hsa-miR-520a-3p | AAAGUGCUUCCCUUUGGACUGU | 465 |
| hsa-miR-520a-5p | CUCCAGAGGGAAGUACUUUCU | 466 |
| hsa-miR-520b | AAAGUGCUUCCUUUUAGAGGG | 467 |
| hsa-miR-520c-3p | AAAGUGCUUCCUUUUAGAGGGU | 468 |
| hsa-miR-520c-5p | CUCUAGAGGGAAGCACUUUCUG | 469 |
| hsa-miR-520d-3p | AAAGUGCUUCUCUUUGGUGGGU | 470 |
| hsa-miR-520d-5p | CUACAAAGGGAAGCCCUUUC | 471 |
| hsa-miR-520e | AAAGUGCUUCCUUUUUGAGGG | 472 |
| hsa-miR-520f | AAGUGCUUCCUUUUAGAGGGUU | 473 |
| hsa-miR-520g | ACAAAGUGCUUCCCUUUAGAGUGU | 474 |
| hsa-miR-520h | ACAAAGUGCUUCCCUUUAGAGU | 475 |
| hsa-miR-521 | AACGCACUUCCCUUUAGAGUGU | 476 |
| hsa-miR-522 | AAAAUGGUUCCCUUUAGAGUGU | 477 |
| hsa-miR-522* | CUCUAGAGGGAAGCGCUUUCUG | 478 |
| hsa-miR-523 | GAACGCGCUUCCCUAUAGAGGGU | 479 |
| hsa-miR-523* | CUCUAGAGGGAAGCGCUUUCUG | 480 |
| hsa-miR-524-3p | GAAGGCGCUUCCCUUUGGAGU | 481 |
| hsa-miR-524-5p | CUACAAAGGGAAGCACUUUCUC | 482 |
| hsa-miR-525-3p | GAAGGCGCUUCCCUUUAGAGCG | 483 |
| hsa-miR-525-5p | CUCCAGAGGGAUGCACUUUCU | 484 |
| hsa-miR-526a | CUCUAGAGGGAAGCACUUUCUG | 485 |
| hsa-miR-526b | CUCUUGAGGGAAGCACUUUCUGU | 486 |
| hsa-miR-526b* | GAAAGUGCUUCCUUUUAGAGGC | 487 |
| hsa-miR-527 | CUGCAAAGGGAAGCCCUUUC | 488 |
| hsa-miR-532-3p | CCUCCCACACCCAAGGCUUGCA | 489 |
| hsa-miR-532-5p | CAUGCCUUGAGUGUAGGACCGU | 490 |
| hsa-miR-539 | GGAGAAAUUAUCCUUGGUGUGU | 491 |
| hsa-miR-541 | UGGUGGGCACAGAAUCUGGACU | 492 |
| hsa-miR-541* | AAAGGAUUCUGCUGUCGGUCCCACU | 493 |
| hsa-miR-542-3p | UGUGACAGAUUGAUAACUGAAA | 494 |
| hsa-miR-542-5p | UCGGGGAUCAUCAUGUCACGAGA | 495 |
| hsa-miR-543 | AAACAUUCGCGGUGCACUUCUU | 496 |
| hsa-miR-544 | AUUCUGCAUUUUUAGCAAGUUC | 497 |
| hsa-miR-545 | UCAGCAAACAUUUAUUGUGUGC | 498 |
| hsa-miR-545* | UCAGUAAAUGUUUAUUAGAUGA | 499 |
| hsa-miR-548a-3p | CAAAACUGGCAAUUACUUUUGC | 500 |
| hsa-miR-548a-5p | AAAAGUAAUUGCGAGUUUUACC | 501 |
| hsa-miR-548b-3p | CAAGAACCUCAGUUGCUUUUGU | 502 |
| hsa-miR-548b-5p | AAAAGUAAUUGUGGUUUUGGCC | 503 |
| hsa-miR-548c-3p | CAAAAAUCUCAAUUACUUUUGC | 504 |
| hsa-miR-548c-5p | AAAAGUAAUUGCGGUUUUUGCC | 505 |
| hsa-miR-548d-3p | CAAAAACCACAGUUUCUUUUGC | 506 |
| hsa-miR-548d-5p | AAAAGUAAUUGUGGUUUUUGCC | 507 |
| hsa-miR-549 | UGACAACUAUGGAUGAGCUCU | 508 |
| hsa-miR-550 | AGUGCCUGAGGGAGUAAGAGCCC | 509 |
| hsa-miR-550* | UGUCUUACUCCCUCAGGCACAU | 510 |
| hsa-miR-551a | GCGACCCACUCUUGGUUUCCA | 511 |
| hsa-miR-551b | GCGACCCAUACUUGGUUUCAG | 512 |
| hsa-miR-551b* | GAAAUCAAGCGUGGGUGAGACC | 513 |
| hsa-miR-552 | AACAGGUGACUGGUUAGACAA | 514 |
| hsa-miR-553 | AAAACGGUGAGAUUUUGUUUU | 515 |
| hsa-miR-554 | GCUAGUCCUGACUCAGCCAGU | 516 |
| hsa-miR-555 | AGGGUAAGCUGAACCUCUGAU | 517 |
| hsa-miR-556-3p | AUAUUACCAUUAGCUCAUCUUU | 518 |
| hsa-miR-556-5p | GAUGAGCUCAUUGUAAUAUGAG | 519 |
| hsa-miR-557 | GUUUGCACGGGUGGGCCUUGUCU | 520 |
| hsa-miR-558 | UGAGCUGCUGUACCAAAAU | 521 |
| hsa-miR-559 | UAAAGUAAAUAUGCACCAAAA | 522 |
| hsa-miR-560 | GCGUGCGCCGGCCGGCCGCC | 523 |
| hsa-miR-561 | CAAAGUUUAAGAUCCUUGAAGU | 524 |
| hsa-miR-562 | AAAGUAGCUGUACCAUUUGC | 525 |
| hsa-miR-563 | AGGUUGACAUACGUUUCCC | 526 |
| hsa-miR-564 | AGGCACGGUGUCAGCAGGC | 527 |
| hsa-miR-565 | GGCUGGCUCGCGAUGUCUGUUU | 528 |
| hsa-miR-566 | GGGCGCCUGUGAUCCCAAC | 529 |
| hsa-miR-567 | AGUAUGUUCUUCCAGGACAGAAC | 530 |
| hsa-miR-568 | AUGUAUAAAUGUAUACACAC | 531 |
| hsa-miR-569 | AGUUAAUGAAUCCUGGAAAGU | 532 |
| hsa-miR-570 | CGAAAACAGCAAUUACCUUUGC | 533 |
| hsa-miR-571 | UGAGUUGGCCAUCUGAGUGAG | 534 |
| hsa-miR-572 | GUCCGCUCGGCGGUGGCCCA | 535 |
| hsa-miR-573 | CUGAAGUGAUGUGUAACUGAUCAG | 536 |
| hsa-miR-574-3p | CACGCUCAUGCACACACCCACA | 537 |
| hsa-miR-574-5p | UGAGUGUGUGUGUGUGAGUGUGU | 538 |
| hsa-miR-575 | GAGCCAGUUGGACAGGAGC | 539 |
| hsa-miR-576-3p | AAGAUGUGGAAAAAUUGGAAUC | 540 |
| hsa-miR-576-5p | AUUCUAAUUUCUCCACGUCUUU | 541 |
| hsa-miR-577 | UAGAUAAAAUAUUGGUACCUG | 542 |
| hsa-miR-578 | CUUCUUGUGCUCUAGGAUUGU | 543 |
| hsa-miR-579 | UUCAUUUGGUAUAAACCGCGAUU | 544 |
| hsa-miR-580 | UUGAGAAUGAUGAAUCAUUAGG | 545 |
| hsa-miR-581 | UCUUGUGUUCUCUAGAUCAGU | 546 |
| hsa-miR-582-3p | UAACUGGUUGAACAACUGAACC | 547 |
| hsa-miR-582-5p | UUACAGUUGUUCAACCAGUUACU | 548 |
| hsa-miR-583 | CAAAGAGGAAGGUCCCAUUAC | 549 |
| hsa-miR-584 | UUAUGGUUUGCCUGGGACUGAG | 550 |
| hsa-miR-585 | UGGGCGUAUCUGUAUGCUA | 551 |
| hsa-miR-586 | UAUGCAUUGUAUUUUUAGGUCC | 552 |
| hsa-miR-587 | UUUCCAUAGGUGAUGAGUCAC | 553 |
| hsa-miR-588 | UUGGCCACAAUGGGUUAGAAC | 554 |
| hsa-miR-589 | UGAGAACCACGUCUGCUCUGAG | 555 |
| hsa-miR-589* | UCAGAACAAAUGCCGGUUCCCAGA | 556 |
| hsa-miR-590-3p | UAAUUUUAUGUAUAAGCUAGU | 557 |
| hsa-miR-590-5p | GAGCUUAUUCAUAAAAGUGCAG | 558 |
| hsa-miR-591 | AGACCAUGGGUUCUCAUUGU | 559 |
| hsa-miR-592 | UUGUGUCAAUAUGCGAUGAUGU | 560 |
| hsa-miR-593 | UGUCUCUGCUGGGGUUUCU | 561 |
| hsa-miR-593* | AGGCACCAGCCAGGCAUUGCUCAGC | 562 |
| hsa-miR-595 | GAAGUGUGCCGUGGUGUGUCU | 563 |
| hsa-miR-596 | AAGCCUGCCCGGCUCCUCGGG | 564 |
| hsa-miR-597 | UGUGUCACUCGAUGACCACUGU | 565 |
| hsa-miR-598 | UACGUCAUCGUUGUCAUCGUCA | 566 |
| hsa-miR-599 | GUUGUGUCAGUUUAUCAAAC | 567 |
| hsa-miR-600 | ACUUACAGACAAGAGCCUUGCUC | 568 |
| hsa-miR-601 | UGGUCUAGGAUUGUUGGAGGAG | 569 |
| hsa-miR-602 | GACACGGGCGACAGCUGCGGCCC | 570 |
| hsa-miR-603 | CACACACUGCAAUUACUUUUGC | 571 |
| hsa-miR-604 | AGGCUGCGGAAUUCAGGAC | 572 |
| hsa-miR-605 | UAAAUCCCAUGGUGCCUUCUCCU | 573 |
| hsa-miR-606 | AAACUACUGAAAAUCAAAGAU | 574 |
| hsa-miR-607 | GUUCAAAUCCAGAUCUAUAAC | 575 |
| hsa-miR-608 | AGGGGUGGUGUUGGGACAGCUCCGU | 576 |
| hsa-miR-609 | AGGGUGUUUCUCUCAUCUCU | 577 |
| hsa-miR-610 | UGAGCUAAAUGUGUGCUGGGA | 578 |
| hsa-miR-611 | GCGAGGACCCCUCGGGGUCUGAC | 579 |
| hsa-miR-612 | GCUGGGCAGGGCUUCUGAGCUCCUU | 580 |
| hsa-miR-613 | AGGAAUGUUCCUUCUUUGCC | 581 |
| hsa-miR-614 | GAACGCCUGUUCUUGCCAGGUGG | 582 |
| hsa-miR-615-3p | UCCGAGCCUGGGUCUCCCUCUU | 583 |
| hsa-miR-615-5p | GGGGGUCCCCGGUGCUCGGAUC | 584 |
| hsa-miR-616 | AGUCAUUGGAGGGUUUGAGCAG | 585 |
| hsa-miR-616* | ACUCAAAACCCUUCAGUGACUU | 586 |
| hsa-miR-617 | AGACUUCCCAUUUGAAGGUGGC | 587 |
| hsa-miR-618 | AAACUCUACUUGUCCUUCUGAGU | 588 |
| hsa-miR-619 | GACCUGGACAUGUUUGUGCCCAGU | 589 |
| hsa-miR-620 | AUGGAGAUAGAUAUAGAAAU | 590 |
| hsa-miR-621 | GGCUAGCAACAGCGCUUACCU | 591 |
| hsa-miR-622 | ACAGUCUGCUGAGGUUGGAGC | 592 |
| hsa-miR-623 | AUCCCUUGCAGGGGCUGUUGGGU | 593 |
| hsa-miR-624 | CACAAGGUAUUGGUAUUACCU | 594 |
| hsa-miR-624* | UAGUACCAGUACCUUGUGUUCA | 595 |
| hsa-miR-625 | AGGGGGAAAGUUCUAUAGUCC | 596 |
| hsa-miR-625* | GACUAUAGAACUUUCCCCCUCA | 597 |
| hsa-miR-626 | AGCUGUCUGAAAAUGUCUU | 598 |
| hsa-miR-627 | GUGAGUCUCUAAGAAAAGAGGA | 599 |
| hsa-miR-628-3p | UCUAGUAAGAGUGGCAGUCGA | 600 |
| hsa-miR-628-5p | AUGCUGACAUAUUUACUAGAGG | 601 |
| hsa-miR-629 | UGGGUUUACGUUGGGAGAACU | 602 |
| hsa-miR-629* | GUUCUCCCAACGUAAGCCCAGC | 603 |
| hsa-miR-630 | AGUAUUCUGUACCAGGGAAGGU | 604 |
| hsa-miR-631 | AGACCUGGCCCAGACCUCAGC | 605 |
| hsa-miR-632 | GUGUCUGCUUCCUGUGGGA | 606 |
| hsa-miR-633 | CUAAUAGUAUCUACCACAAUAAA | 607 |
| hsa-miR-634 | AACCAGCACCCCAACUUUGGAC | 608 |
| hsa-miR-635 | ACUUGGGCACUGAAACAAUGUCC | 609 |
| hsa-miR-636 | UGUGCUUGCUCGUCCCGCCCGCA | 610 |
| hsa-miR-637 | ACUGGGGGCUUUCGGGCUCUGCGU | 611 |
| hsa-miR-638 | AGGGAUCGCGGGCGGGUGGCGGCCU | 612 |
| hsa-miR-639 | AUCGCUGCGGUUGCGAGCGCUGU | 613 |
| hsa-miR-640 | AUGAUCCAGGAACCUGCCUCU | 614 |
| hsa-miR-641 | AAAGACAUAGGAUAGAGUCACCUC | 615 |
| hsa-miR-642 | GUCCCUCUCCAAAUGUGUCUUG | 616 |
| hsa-miR-643 | ACUUGUAUGCUAGCUCAGGUAG | 617 |
| hsa-miR-644 | AGUGUGGCUUUCUUAGAGC | 618 |
| hsa-miR-645 | UCUAGGCUGGUACUGCUGA | 619 |
| hsa-miR-646 | AAGCAGCUGCCUCUGAGGC | 620 |
| hsa-miR-647 | GUGGCUGCACUCACUUCCUUC | 621 |
| hsa-miR-648 | AAGUGUGCAGGGCACUGGU | 622 |
| hsa-miR-649 | AAACCUGUGUUGUUCAAGAGUC | 623 |
| hsa-miR-650 | AGGAGGCAGCGCUCUCAGGAC | 624 |
| hsa-miR-651 | UUUAGGAUAAGCUUGACUUUUG | 625 |
| hsa-miR-652 | AAUGGCGCCACUAGGGUUGUG | 626 |
| hsa-miR-653 | GUGUUGAAACAAUCUCUACUG | 627 |
| hsa-miR-654-3p | UAUGUCUGCUGACCAUCACCUU | 628 |
| hsa-miR-654-5p | UGGUGGGCCGCAGAACAUGUGC | 629 |
| hsa-miR-655 | AUAAUACAUGGUUAACCUCUUU | 630 |
| hsa-miR-656 | AAUAUUAUACAGUCAACCUCU | 631 |
| hsa-miR-657 | GGCAGGUUCUCACCCUCUCUAGG | 632 |
| hsa-miR-658 | GGCGGAGGGAAGUAGGUCCGUUGGU | 633 |
| hsa-miR-659 | CUUGGUUCAGGGAGGGUCCCCA | 634 |
| hsa-miR-660 | UACCCAUUGCAUAUCGGAGUUG | 635 |
| hsa-miR-661 | UGCCUGGGUCUCUGGCCUGCGCGU | 636 |
| hsa-miR-662 | UCCCACGUUGUGGCCCAGCAG | 637 |
| hsa-miR-663 | AGGCGGGGCGCCGCGGGACCGC | 638 |
| hsa-miR-665 | ACCAGGAGGCUGAGGCCCCU | 639 |
| hsa-miR-668 | UGUCACUCGGCUCGGCCCACUAC | 640 |
| hsa-miR-671-3p | UCCGGUUCUCAGGGCUCCACC | 641 |
| hsa-miR-671-5p | AGGAAGCCCUGGAGGGGCUGGAG | 642 |
| hsa-miR-672 | UGAGGUUGGUGUACUGUGUGUGA | 643 |
| hsa-miR-674 | GCACUGAGAUGGGAGUGGUGUA | 644 |
| hsa-miR-675 | UGGUGCGGAGAGGGCCCACAGUG | 645 |
| hsa-miR-7 | UGGAAGACUAGUGAUUUUGUUGU | 646 |
| hsa-miR-708 | AAGGAGCUUACAAUCUAGCUGGG | 647 |
| hsa-miR-708* | CAACUAGACUGUGAGCUUCUAG | 648 |
| hsa-miR-7-1* | CAACAAAUCACAGUCUGCCAUA | 649 |
| hsa-miR-7-2* | CAACAAAUCCCAGUCUACCUAA | 650 |
| hsa-miR-744 | UGCGGGGCUAGGGCUAACAGCA | 651 |
| hsa-miR-744* | CUGUUGCCACUAACCUCAACCU | 652 |
| hsa-miR-758 | UUUGUGACCUGGUCCACUAACC | 653 |
| hsa-miR-760 | CGGCUCUGGGUCUGUGGGGA | 654 |
| hsa-miR-765 | UGGAGGAGAAGGAAGGUGAUG | 655 |
| hsa-miR-766 | ACUCCAGCCCCACAGCCUCAGC | 656 |
| hsa-miR-767-3p | UCUGCUCAUACCCCAUGGUUUCU | 657 |
| hsa-miR-767-5p | UGCACCAUGGUUGUCUGAGCAUG | 658 |
| hsa-miR-768-3p | UCACAAUGCUGACACUCAAACUGCUGAC | 659 |
| hsa-miR-768-5p | GUUGGAGGAUGAAAGUACGGAGUGAU | 660 |
| hsa-miR-769-3p | CUGGGAUCUCCGGGGUCUUGGUU | 661 |
| hsa-miR-769-5p | UGAGACCUCUGGGUUCUGAGCU | 662 |
| hsa-miR-770-5p | UCCAGUACCACGUGUCAGGGCCA | 663 |
| hsa-miR-801 | GAUUGCUCUGCGUGCGGAAUCGAC | 664 |
| hsa-miR-802 | CAGUAACAAAGAUUCAUCCUUGU | 665 |
| hsa-miR-871 | UAUUCAGAUUAGUGCCAGUCAUG | 666 |
| hsa-miR-872 | AAGGUUACUUGUUAGUUCAGG | 667 |
| hsa-miR-873 | GCAGGAACUUGUGAGUCUCCU | 668 |
| hsa-miR-874 | CUGCCCUGGCCCGAGGGACCGA | 669 |
| hsa-miR-875-3p | CCUGGAAACACUGAGGUUGUG | 670 |
| hsa-miR-875-5p | UAUACCUCAGUUUUAUCAGGUG | 671 |
| hsa-miR-876-3p | UGGUGGUUUACAAAGUAAUUCA | 672 |
| hsa-miR-876-5p | UGGAUUUCUUUGUGAAUCACCA | 673 |
| hsa-miR-877 | GUAGAGGAGAUGGCGCAGGG | 674 |
| hsa-miR-877* | UCCUCUUCUCCCUCCUCCCAGG | 675 |
| hsa-miR-885-3p | AGGCAGCGGGGUGUAGUGGAUA | 676 |
| hsa-miR-885-5p | UCCAUUACACUACCCUGCCUCU | 677 |
| hsa-miR-886-3p | CGCGGGUGCUUACUGACCCUU | 678 |
| hsa-miR-886-5p | CGGGUCGGAGUUAGCUCAAGCGG | 679 |
| hsa-miR-887 | GUGAACGGGCGCCAUCCCGAGG | 680 |
| hsa-miR-888 | UACUCAAAAAGCUGUCAGUCA | 681 |
| hsa-miR-888* | GACUGACACCUCUUUGGGUGAA | 682 |
| hsa-miR-889 | UUAAUAUCGGACAACCAUUGU | 683 |
| hsa-miR-890 | UACUUGGAAAGGCAUCAGUUG | 684 |
| hsa-miR-891a | UGCAACGAACCUGAGCCACUGA | 685 |
| hsa-miR-891b | UGCAACUUACCUGAGUCAUUGA | 686 |
| hsa-miR-892a | CACUGUGUCCUUUCUGCGUAG | 687 |
| hsa-miR-892b | CACUGGCUCCUUUCUGGGUAGA | 688 |
| hsa-miR-9 | UCUUUGGUUAUCUAGCUGUAUGA | 689 |
| hsa-miR-9* | AUAAAGCUAGAUAACCGAAAGU | 690 |
| hsa-miR-920 | GGGGAGCUGUGGAAGCAGUA | 691 |
| hsa-miR-921 | CUAGUGAGGGACAGAACCAGGAUUC | 692 |
| hsa-miR-922 | GCAGCAGAGAAUAGGACUACGUC | 693 |
| hsa-miR-923 | GUCAGCGGAGGAAAAGAAACU | 694 |
| hsa-miR-924 | AGAGUCUUGUGAUGUCUUGC | 695 |
| hsa-miR-92a | UAUUGCACUUGUCCCGGCCUGU | 696 |
| hsa-miR-92a-1* | AGGUUGGGAUCGGUUGCAAUGCU | 697 |
| hsa-miR-92a-2* | GGGUGGGGAUUUGUUGCAUUAC | 698 |
| hsa-miR-92b | UAUUGCACUCGUCCCGGCCUCC | 699 |
| hsa-miR-92b* | AGGGACGGGACGCGGUGCAGUG | 700 |
| hsa-miR-93 | CAAAGUGCUGUUCGUGCAGGUAG | 701 |
| hsa-miR-93* | ACUGCUGAGCUAGCACUUCCCG | 702 |
| hsa-miR-933 | UGUGCGCAGGGAGACCUCUCCC | 703 |
| hsa-miR-934 | UGUCUACUACUGGAGACACUGG | 704 |
| hsa-miR-935 | CCAGUUACCGCUUCCGCUACCGC | 705 |
| hsa-miR-936 | ACAGUAGAGGGAGGAAUCGCAG | 706 |
| hsa-miR-937 | AUCCGCGCUCUGACUCUCUGCC | 707 |
| hsa-miR-938 | UGCCCUUAAAGGUGAACCCAGU | 708 |
| hsa-miR-939 | UGGGGAGCUGAGGCUCUGGGGGUG | 709 |
| hsa-miR-940 | AAGGCAGGGCCCCCGCUCCCC | 710 |
| hsa-miR-941 | CACCCGGCUGUGUGCACAUGUGC | 711 |
| hsa-miR-942 | UCUUCUCUGUUUUGGCCAUGUG | 712 |
| hsa-miR-943 | CUGACUGUUGCCGUCCUCCAG | 713 |
| hsa-miR-944 | AAAUUAUUGUACAUCGGAUGAG | 714 |
| hsa-miR-95 | UUCAACGGGUAUUUAUUGAGCA | 715 |
| hsa-miR-96 | UUUGGCACUAGCACAUUUUUGCU | 716 |
| hsa-miR-96* | AAUCAUGUGCAGUGCCAAUAUG | 717 |
| hsa-miR-98 | UGAGGUAGUAAGUUGUAUUGUU | 718 |
| hsa-miR-99a | AACCCGUAGAUCCGAUCUUGUG | 719 |
| hsa-miR-99a* | CAAGCUCGCUUCUAUGGGUCUG | 720 |
| hsa-miR-99b | CACCCGUAGAACCGACCUUGCG | 721 |
| hsa-miR-99b* | CAAGCUCGUGUCUGUGGGUCCG | 722 |
| hsv-1miR-LAT | UGGCGGCCCGGCCCGGGGCC | 723 |

Sequences are shown from the 5′ to 3′ direction.

In still another embodiment, the seed sequence of the micro RNA is selected from the group consisting of position 1-20, position 1-19, position 1-18, position 1-17, position 1-16, position 1-15, position 1-14, position 1-13, position 1-12, position 1-11, position 1-10, position 1-9, position 1-8, position 1-7, position 1-6, position 2-20, position 2-19, position 2-18, position 2-17, position 2-16, position 2-15, position 2-14, position 2-13, position 2-12, position 2-11, position 2-10, position 2-9, position 2-8, position 2-7, position 2-6, position 3-20, position 3-19, position 3-18, position 3-17, position 3-16, position 3-15, position 3-14, position 3-13, position 3-12, position 3-11, position 3-10 and position 3-9 of any SEQ ID NOs:1-723.

In a more preferred embodiment, the seed sequence of the micro RNA is selected from the group consisting of: position 1-10, position 1-9, position 1-8, position 1-7, position 1-6, position 2-10, position 2-9, position 2-8, position 2-7, position 2-6, position 3-10 and position 3-9 of any SEQ ID NOs:1-723.

In a most preferred embodiment, the seed sequence of the micro RNA is selected from the group consisting of: position 1-8, position 1-7, position 2-8 and position 2-7 of any SEQ ID NOs: 1-723.

### Activity of the Oligonucleotide of the Invention

As will be clear, the oligonucleotides of the invention have a variety of utilities and advantages.

### RNase H Cleavage

In one embodiment, the oligonucleotide draws use of the accessibility of a target region of a target RNA. In this embodiment, the oligonucleotide may activate RNase H cleavage of the target. Because of the improved target accessibility, the oligonucleotide will preferentially affect the activity of the target RNA, even if the oligonucleotide is short, e.g. about 10 bases or just the guide sequence. I.e. complementary regions elsewhere may not be targeted because they are less accessible. They may e.g. be buried in RNA secondary structure or may be inaccessible because they are engaged in protein binding.

RNase H will cleave the RNA part of a RNA-DNA duplex. The structural requirements for RNase H activation are well-known to the skilled man. This mechanism is very often used to achieve traditional antisense regulation e.g. by employing so-called gapmers. Gapmers are antisense oligonucleotides that comprise a central region with deoxy sugars (the gap) and modified flanks. Gapmers very often comprises phosphorothioate internucleotide linkages to improve biostability and the flanks comprise e.g. 2-O-modifications that also improve biostability, i.e. resistance against nucleolytic attack. The flanks may also comprise modifications that increase the melting temperature of the gapmer base paired to a complementary nucleic acid. Also headmer and endmer structures have been described in the literature.

In another preferred embodiment, the oligonucleotide is not capable of inducing RNase H cleavage of the target RNA. The skilled man is well aware of the requirements for RNase H cleavage and will be able to design oligonucleotides that do or do not activate RNase H.

Thus, in a preferred embodiment, the oligonucleotide does not comprise a stretch of unmodified DNA that exceeds a length selected from the group consisting of: 3 bases, 4 bases, 5 bases, 6 bases, 7 bases, 8 bases, 9 bases, 10 bases and 11 bases. Most preferably, the stretch of unmodified DNA does not exceed 3 bases.

In another preferred embodiment, the oligonucleotide does not comprise any DNA monomers.

### Recruiting the RNAi Machinery

The RNAi machinery is a sophisticated gene regulatory system that is guided by RNA. Thus, microRNAs guide the RNAi machinery to target mRNAs to affect the activity of the target mRNA. The RNAi machinery may affect translation of the mRNA directly or it may affect the stability of the target mRNA, i.e. mediate direct degradation of the target mRNA. Not intended to be bound by theory, it is believed that the degree of complementarity between microRNA and target mRNA is a key element as to whether the target mRNA is subjected to translational regulation or degradation.

Endogenous microRNAs are processed from precursor stem-loops and incorporated into a so called RNA induced silencing complex (RISC complex). The details of this process are still poorly understood.

The cellular RNAi machinery has been extensively used to affect the activity of cellular mRNAs by introducing synthetic double stranded RNA complexes termed siRNAs into the cell. As mentioned above, siRNAs are short double stranded RNA complexes comprising a passenger strand and a complementary guide strand. The guide strand of siRNA is incorporated into the RISC complex, where after the RISC complex can affect the activity of mRNA harbouring complementary sequences to the guide strand. Thus, siRNAs are a new class of compounds that is thought to be capable of efficiently and specifically targeting any mRNA and consequently, siRNAs are regarded potentially as a new class of therapeutics.

A common feature of siRNAs and microRNAs is that they recruit the cellular RNAi complex to affect the activity of target RNAs.

In one embodiment, the oligonucleotides of the invention are capable of recruiting the RNAi machinery and hence direct the RNAi machinery to the target RNA. This may result in cleavage of the target RNA or translational repression of the target RNA. In this embodiment, the oligonucleotide may be a siRNA. I.e. the oligonucleotide is hybridised to a complementary oligonucleotide, typically over a length of 20-22 bases and very often with 3′overhangs of 1-3 bases. As the name implies, a siRNA essentially consists of RNA monomers, although modifications, such as e.g. 2′-O-modifications are acceptable at certain positions.

The oligonucleotide may also act as a microRNA, without being identical to a naturally occurring microRNA. When the oligonucleotide acts as a microRNA, it consists essentially of RNA monomers, although modifications may be acceptable at certain positions. The oligonucleotide may have a structure analogously to a mature endogenous microRNA or to a pre-microRNA (stem-loop with bulges in stem) that has to be processed by dicer to a mature microRNA.

Where naturally occurring microRNAs typically regulate many target RNAs, a oligonucleotide of the invention acting as a microRNA may be designed to only regulate a few target RNAs or only one target RNA. Promiscuity of the oligonucleotide can be adjusted by designing the oligonucleotide to target only one or a few targets. By using universal bases, a large degree of promiscuity can also be designed into the oligonucleotide. Universal bases will be discussed more below. Promiscouity can also be introduced by reducing the length of the oligonucleotide.

Importantly, when the oligonucleotides of the invention are capable of recruiting the RNAi machinery, they may still draw use of the accessibility of the target region of the target RNA.

### Blockmir

In another embodiment, the oligonucleotides cannot recruit the RNAi machinery. In this embodiment, it is preferred that the oligonucleotides of the invention are capable of blocking the activity of the RNAi machinery at a particular target RNA. As mentioned above, the oligonucleotides may do so by sequestering the target sequence of the target RNA, such that the RNAi machinery will not recognize the target sequence, as it is base paired to the oligonucleotides. Oligonucleotides of the invention with this activity may also be referred to as blockmirs.

In a preferred embodiment, the oligonucleotide is capable of blocking the regulatory activity of a microRNA at a particular target RNA. Preferably, the microRNA is an endogenous microRNA.

After the priority date of this patent application, examples of oligonucleotides capable of blocking the regulatory activity of a microRNA at a given mRNA has been published by two groups.

In the first publication (Xiao J, 2007), oligonucleotides termed microRNA masking antisense ODN (oligodeoxynucleotides) was used to interfere with the regulatory activity of mir-1 on HCN2 and HCN4 and the regulatory activity of mir-133 on HCN2. It was observed that microRNA masking antisense increased the protein level of HCN2 and HCN4 in a gene specific manner, as determined by immunoblotting using cultured neonatal rat ventricular cells and luciferase assays using HEK293 human embryonic kidney cell line. I.e. the mechanism of action of blockmirs was validated. In other words, it was demonstrated that an oligonucleotide that binds to the target site of a microRNA in the 3′UTR of a mRNA, can prevent microRNA regulation of the mRNA in mammalian cells (rat and human).

However, the design of the blockmirs in the work of Xiao et al., 2007 left some questions open. The microRNA masking antisense ODN consisted of deoxynucleotides with 5 LNA monomers at both ends. Thus, the central part of the oligonucleotide apparently consisted of a stretch of 12 unmodified deoxynucleotides. Such structure is typically expected to activate RNase H and hence mediate degradation of target RNAs.

In the second publication (Choi W Y, 2007) blockmirs (termed target protectors) was used to prevent microRNA regulation of specific mRNAs in zebrafish. More specifically, the authors used morpholino oligonucleotides of 25 units with perfect complementarity to zebrafish mir-430 target sites in squint and lefty mRNA to prevent mir-430 regulation of the target mRNAs (squint and lefty). Thus, the authors validate the blockmir approach in a different organism than did Xiao et al., and they also validate that a different chemistry can be used.

We suggest that the essence of blockmir activity is binding to a microRNA target site, and that this can achieved using a variety of chemistries and also in a variety of organisms.

Another report published after the priority date of this patent application studied the molecular basis for target RNA recognition and cleavage by human RISC (Ameres S L, 2007). These authors found that target accessibility determines RISC mediated cleavage in vitro and in vivo. Among others, they blocked target accessibility using oligonucleotides complementary to a siRNA target site, i.e. the oligonucleotides may be seen as functional analogues of the blockmirs of the present invention, except that they target a siRNA target site that is regulated by a siRNA with perfect complementary. Interestingly, the authors found that blocking 3 or 6 nt of the 21 nt target sequence in the region annealing to the 3′part of the siRNA had no effect on regulation (as seen by cleavage rates using affinity purified human RISC). In contrast, blocking 5 nt of the target site in the region annealing to the 5′part of the siRNA severely impaired regulation (as seen by cleavage) and even blocking only 2 nt impaired regulation.

Returning to blockmirs of the invention, if the microRNA is a positive regulator of the target RNA, the oligonucleotide will be a negative regulator of the target RNA.

Most often, the microRNA is a negative regulator of the target RNA. Thus, in another embodiment, the oligonucleotide is a positive regulator of the target RNA. This is contrary to traditional antisense oligonucleotides, microRNAs and siRNAs that typically act as negative regulators.

In a preferred embodiment, the blockmirs of the invention are DNAs, as these will not be recognized by the RNAi machinery and consequently function as neither microRNA nor siRNA. Preferably, the DNA units are modified such as to prevent RNase H activation. Alternatively, less than 5 consecutive DNA units are present, such as less than 4 consecutive DNA and less than 3 consecutive DNA units.

In still another embodiment, the blockmir does not comprise any DNA units.

In yet another embodiment, the blockmir does not comprise any RNA units.

In another embodiment, the blockmir does not comprise a stretch of RNA units that exceeds a length selected from the group of consisting of: a length of 5 units, 6 units, 7 units, 8 units, 9 units, 10 units, 11 units, 12 units, 13 units, 14 units, 15 units, 16 units, 17 units, 18 units, 19 units, 20 units, 21 units and 22 units.

In one embodiment, the oligonucleotides have been chemically modified such as to not being capable of recruiting the RNAi machinery. Preferred modifications include 2′-O-modications such as 2′-O-methyl and 2′O-F. Also conjugated RNAs are preferred. E.g. RNAs conjugated to a cholesterol moiety, in which case the cholesterol may both prevent the oligonucleotide from recruiting the RNAi machinery and improve the bioavailability of the oligonucleotide. The cholesterol moiety may be conjugated to a monomer within the guide sequence of the oligonucleotide or at the 3′end or the 5′end of the oligonucleotide. More modifications are described below.

In yet another embodiment, the blockmir may comprise a mix of DNA units and RNA units such as to prevent the oligonucleotide from activating RNase H and to at the same time prevent the oligonucleotide from recruiting the RNAi machinery. E.g. a DNA unit may be followed by a RNA unit that is again followed by a DNA unit and so on. Further, in a preferred embodiment, phosphorothioate internucleotide linkages may connect the units to improve the biostability of the oligonucleotide. Both DNA units and RNA units may be modified. Preferably, RNA units are modified in the 2′-O-position (2′-O-methyl, LNA etc.).

In yet another embodiment, the oligonucleotide (blockmir) comprise a mix of DNA units and RNA units such as to prevent the oligonucleotide from activating RNase H and to at the same time prevent the oligonucleotide from recruiting the RNAi machinery, wherein the DNA units and RNA units come in blocks. The blocks may have a length of 2 units, 3 units, 4 units, 5 units or 6 units and units of different length may be comprised with the same oligonucleotide. Both DNA units and RNA units may be modified. Preferably, RNA units are modified in the 2′-O-position (2′-O-methyl, LNA etc).

In a preferred embodiment, also units selected from the group of LNA units, INA units and morpholino units are comprised within the oligonucleotide. In another preferred embodiment, the oligonucleotide comprises a mix of LNA units and RNA units with a 2′-O-methyl. Such mixmers have been used as steric block inhibitors of Human Immunodeficiency Virus Type 1 Tat-Dependent Trans-Activation and HIV-1 Infectivity.

In still another embodiment, the blockmir are entirely composed of units selected from the group of 2′-O-methyl modified units, LNA units, PNA units, INA units and morpholino units. In one embodiment, the units are mixed, while in another embodiment, the blockmir is composed of only one of the units.

In still another embodiment, the blockmir has been designed such as to able to bind to more than one target RNA. Promiscuity may be designed into blockmirs using universal bases. Also reducing the length of the blockmir will increase promiscuity. Thus, in one embodiment, the blockmir may only consist of the guide sequence corresponding to a seed sequence of a microRNA. In this embodiment, it is preferred that affinity increasing modifications are used and the oligonucleotide may be fully modified in the 2′O-position with e.g. 2′-O-methyl, 2′-O-′flouro, 2′-0-(2-methoxyethyl) or the nucleotides may be locked (LNA).

### Off-Target Effects

In most embodiments, off-target binding of the blockmir will have very few or no effects. This is contrary to antimirs, RNAi mediated by siRNAs and microRNAs, and RNase H mediated antisense regulation, which may all give rise to off-targets effects. The blockmir only has an effect if it binds to a microRNA target region and thereby prevents microRNA regulation of the target RNA.

Thus, in a preferred embodiment, the blockmir will have reduced off-target effects, as compared to regulating the activity of the target mRNA using an antimir.

An antimir, as used in the present context, is an oligonucleotide that can base pair with a microRNA and thereby inhibit the activity of the microRNA. Since most microRNAs are promiscuous, i.e. they regulate more than one target, regulation of a particular microRNA will affect the activity of more than one target mRNA. Thus, when it is desired to only regulate the activity of one particular target mRNA, regulation of other target mRNAs may be referred to as off-target effects of the antimir.

Using a (exogenous) promiscuous microRNA to affect or regulate the activity of a target mRNA, instead of an antimir may obviously also have off-target effects.

Moreover, the target repertoire of a given microRNA may vary in different cells, wherefore an antimir may have different off target effects in different cells. Likewise for regulation using a promiscuous microRNA. A blockmir will only have an effect in the particular cells wherein the target RNA is regulated by a microRNA. Thus, a blockmir enables targeting of cell specific microRNA:mRNA interactions. If the blockmir enter a cell that does not have the particular microRNA:mRNA interaction, the blockmir will have little or no effect.

siRNAs are double stranded RNA complexes comprising a passenger strand and a guide strand that mediate degradation of target mRNAs that are complementary to the guide strand of the RNA complex. It has now been recognized that siRNAs often have off-target effects, because the strand acting as guide strand can also function as microRNA, i.e. siRNAs may mediate regulation of target mRNAs that are not fully complementary to the guide strand of the siRNA.

Thus, in one embodiment, an blockmir of the present invention will have reduced off-target effects as compared to a siRNA directed to the same target mRNA.

In another preferred embodiment, the blockmir will also have reduced off-target effects as compared to using a traditional antisense oligonucleotide for regulation of the target mRNA.

Traditional antisense oligonucleotides are often designed such as to mediate RNase H cleavage of their target RNA. RNase H cleaves a duplex of RNA and DNA.

Thus, if such an antisense oligonucleotide base pairs to a non-intended mRNA, this mRNA will be inactivated by RNase H cleavage, and hence giving rise to off-target effects.

In conclusion, blockmirs of the present invention are characteristic in that they affect the activity of an RNA by preventing microRNA regulation of the target RNA. Thus, blockmirs of the present invention will have reduced off target effects as compared to both traditional antisense oligonucleotides, antimirs, and RNAi mediated regulation using microRNAs and siRNAs.

A consideration when designing short blockmirs is obviously that the transcriptome may comprise more than one site with perfect complementary to the blockmir. However, as outlined above, the blockmirs will only affect the target RNA if the target sequence is also a target sequence for microRNA regulation. Therefore, even very short blockmirs may have very little of no off-target effects. Thus, the blockmirs may deliberately be designed to target many sites. The blockmirs will then preferentially bind to microRNA target sites since these are more accessible, and the blockmirs will only have effects if they prevent microRNA binding to a target site.

### Chemistry

In a preferred embodiment of the oligonucleotides of the invention, the oligonucleotide comprises nucleotide monomers that increase its affinity for complementary sequences or affinity increasing modifications. This is particular relevant for short oligonucleotides and may allow for generation of very short active oligonucleotides, e.g. of a length between 10 and 15 bases or even less than 10 bases, such as e.g. only the guide sequence corresponding to the seed sequence of a microRNA.

Nucleotide units that increase the affinity for complementary sequences may e.g. be LNA (locked nucleic acid) units, PNA (peptide nucleic acid) units or INA (intercalating nucleic acid) units. Also RNA units modified in the 2-O-position (e.g. 2′-0-(2-methoxyethyl)-RNA, 2′O-methyl-RNA, 2′O-flouro-RNA) increase the affinity for complementary sequences. At the same time, such modifications often also improve the biostability of the oligonucleotides, as they become a poorer substrate for cellular nucleases.

The oligonucleotide may also comprise modifications that increase its biostability and/or bioavailability, such as phosphorothioate linkages. The oligonucleotide may be fully phosphorothiolated or only partly phosphorothiolated.

In a preferred embodiment, the oligonucleotide comprises a repeating pattern of one or more LNA units and one or more units that are substituted in the 2′-position. OMe/LNA mixmers have been shown to be powerful reagents for use as steric block inhibitors of gene expression regulated by protein-RNA interactions. Thus, when the oligonucleotides of the invention are used to block the activity of a microRNA at a target RNA, a OMe/LNA mixmer architecture may be used. A gapmer structure may also be used, however preferably without being capable of inducing RNase H if the oligonucleotide is intended to act as a blockmir.

In one embodiment, the oligonucleotide of the invention does not comprise any RNA units. Few or no RNA units may be used to prevent the oligonucleotide from being capable of recruiting the RNAi machinery. Chemical modifications can do the same.

In another embodiment, the oligonucleotide of the invention does not comprise any DNA units.

In still another embodiment, the oligonucleotide of the invention does not comprise any morpholino units and/or LNA units.

In yet another embodiment, the oligonucleotide comprises modifications that increase its biostability. The modifications may be the nucleotide units mentioned above for increasing the affinity toward complementary sequences.

In a preferred embodiment, the oligonucleotides comprise a number of nucleotide units that increase the affinity for complementary sequences selected from the group of: 1 units, 2 units, 3 units, 4 units, 5 units, 6 units, 7 units, 8 units, 9 units, 10 units, 11 units, 12 units, 13 units, 14 units, 15 units, 16 units, 17 units, 18 units, 19 units, 20 units, 21 units, and 22 units.

In a preferred embodiment, nucleotide units that increase the affinity for complementary sequences are located at the flanks of the oligonucleotide. E.g. if the oligonucleotide comprise e.g. 10 LNA units, 5 may be located at the 5′end and the other 5 units may be located at the 3′end.

In still another embodiment, the oligonucleotides comprise modifications that increase its bioavailability. Modifications that improve cellular delivery are particular preferred.

### Promiscuity and Specificity

In yet another embodiment, the oligonucleotide of the present invention may comprise nucleotides that do not hybridise specifically. Such nucleotides comprise so called universal bases. These are characterised in that they fit into a Watson-crick helix opposite to any base. Thus, they may be used to impose a certain degree of promiscuity on the oligonucleotides of the invention. That may e.g. be employed if the oligonucleotide is intended to target two particular mRNAs.

In a preferred embodiment, it may be desired to target most or all targets of a particular microRNA. In such case, the oligonucleotide may comprise a guide sequence corresponding to the seed sequence of the microRNA and one or two blocks of natural bases. The size of the blocks of natural bases can be adjusted such as to achieve a reasonable affinity to target sequences.

In still another embodiment, the oligonucleotide of the invention comprises a universal base selected from the group consisting of 3-nitropyrrole, 5-nitroindole, 3-methyl isocarbostyril or 5-methyl isocarbostyril.

In one embodiment, the oligonucleotides of the invention may comprise a guide sequence which is flanked by universal bases on the 3′side, the 5′side or both. Such an oligonucleotide may be used to mimic the promiscuous specificity of a microRNA and hence, block the activity of the microRNA at multiple target RNAs or even all target RNAs of the microRNAs. A combination of universal bases and e.g. inosine may also be used to design an oligonucleotide that only targets a subset of the target RNAs of a microRNA.

In one embodiment, the bases between the guide sequence and the second sequence are universal bases.

In another embodiment, any bases not part of the guide sequence and the second sequence are universal bases.

Universal bases tend to decrease the melting temperature of the oligonucleotide, wherefore it is preferred to counteract this decrease by incorporation of affinity increasing modifications or units, e.g. LNA units or 2′-O-methyl groups.

Single-Stranded Vs. Double Stranded

In some embodiments, the oligonucleotide of the invention is preferably not base paired with a complementary oligonucleotide or intended for use with a base paired with a complementary oligonucleotide. I.e. it should be single stranded to facilitate interaction with a target RNA and in certain embodiments, also to prevent recruitment of the RNAi machinery.

In another embodiment, the oligonucleotide is base paired to a complementary oligonucleotide. In some situations, it may be desirable that the oligonucleotide is base paired to a complementary oligonucleotide to facilitate transport into a cell and/or intracellular transport. Also transport within an organism may be facilitated. Further, biostability may be positively affected.

Base pairing to a complementary oligonucleotide will also be used when the oligonucleotide is acting as a siRNA. When the oligonucleotide is acting as a exogenous miRNA, it may be formed as a stem-loop structure.

In another embodiment, the oligonucleotide is base paired to a RNA molecule that is degraded by RNase H, when the oligonucleotide enters its target cell. In this way, the oligonucleotide is liberated on site. In a preferred embodiment, the complementary oligonucleotide is not of the same type as the oligonucleotide of the invention. E.g. if the oligonucleotide is RNA, the complementary oligonucleotide will not be RNA.

### Delivery

Various methods for delivery of oligonucleotides are known to the skilled man. Thus, oligonucleotides may be formulated in microparticles and nanoparticles. Liposomes are frequently used as delivery vehicle and a variety of liposome delivery systems exist. They may e.g. comprise cationic lipids or neutral lipids. Their size may be varied for various purposes and other components may be included in the liposomes or on the surface of the liposomes. Chitosan nanoparticles have been used for delivery of plasmids and siRNAs to various cells, among them primary cells. Thus, chitosan nanoparticles may also be used for delivery of the oligonucleotides of the invention. Others polymers for delivery are polyethyleneimine (PEI), cyclodextrin, atelocollagen, polyamidoamine (PAMAM) and poly(lactic-co-glycolic acid) (PLGA). Further, oligonucleotides of the invention may be conjugated to cationic peptides that have been shown to facilitate transport into cells.

### Second Aspect—Method of Modulating the Activity of a Target RNA

A second aspect of the invention is a method of modulating the activity of a target RNA comprising the steps
a. Providing a system comprising a target RNA
b. Providing an oligonucleotide that comprises an antisense sequence complementary to a target region of the target RNA
c. Introducing the oligonucleotide of step b to the system of step a
d. Thereby modulating the activity of the target RNA

Preferably, the oligonucleotide is an oligonucleotide of the invention, as described in the first aspect of the invention in various embodiments.

And preferably, the target RNA comprises an anti-seed sequence which is complementary to the guide sequence of the oligonucleotide.

In a preferred embodiment, the oligonucleotide prevents the activity of a microRNA at the target RNA and thereby modulates the activity of the target RNA. I.e. the oligonucleotide is a blockmir as described in the first aspect.

In another embodiment, the oligonucleotide induces RNase H cleavage of the target RNA and thereby regulates the activity of the target RNA.

In yet another embodiment, the oligonucleotide recruits the RNAi machinery to the target RNA. Recruitment of the RNAi machinery may lead to translational repression of the target RNA or degradation of the target RNA.

Preferably, the system is either a cell extract or a cell. The method may be performed in vivo, ex vivo or in vitro.

In one embodiment, the method is a method for validating the activity of the oligonucleotide, i.e. verifying whether the oligonucleotide can indeed modulate the activity of the target RNA and to what extent. Such method may be used when aiming to identify oligonucleotides with optimal activity e.g. for therapeutic development. In such testing, typically different lengths and chemistries of the oligonucleotide will be tested.

In another embodiment, the method is a method of identifying or validating a micro RNA target of a target RNA. Very often, it is hypothesized that a microRNA regulates a given target RNA and in this case, the method of the second aspect is a method of verifying whether the target RNA is indeed regulated by a microRNA. Thus, the method may further comprise identifying the microRNA that regulates the target RNA. This is possible because the target RNA should comprise an anti-seed sequence which is complementary the seed sequence of the microRNA.

### Third Aspect—Providing a Bioactive Oligonucleotide

A third aspect of the invention is a method comprising the steps of:
a. Providing a (predetermined) target sequence of a target RNA regulated by a microRNA, said target sequence being the sequence of the target RNA involved in microRNA regulation.
b. Designing an oligonucleotide sequence that comprises a continuous stretch of bases (antisense sequence) of at least 6 bases that is complementary to the target sequence
c. Synthesizing the oligonucleotide sequence of step b, said oligonucleotide being a candidate regulator of the activity of a target RNA.

In a preferred embodiment, the method is a method of providing a bioactive oligonucleotide.

Preferably, the continuous stretch of bases comprises the guide sequence corresponding to the seed sequence of the micro RNA regulating the target RNA.

Preferably, the method further comprises the steps
a. Providing a reporter system for activity of the target RNA
b. Determining the activity of the target RNA in the presence of the candidate regulator
c. Determining the activity of the target RNA in the absence of the candidate regulator
d. Comparing the activity levels in b and c and thereby verifying whether the oligonucleotide is indeed a capable of regulating the activity of the RNA and/or whether the potential target sequence of the RNA is indeed a target sequence.

In yet another preferred embodiment, the method further comprises a step of determining the activity of the target RNA in the presence of a negative control, said negative control being an oligonucleotide that does not have complementarity to any region in the target RNA. In another related embodiment, the negative control is an oligonucleotide which is complementary to the oligonucleotide it serves as a control for. In still another embodiment, the negative control is complementary to a region which is not part of the target region of the target RNA. Preferably, the oligonucleotide and its negative control are of the same type, i.e., RNA, mixed RNA and DNA, and comprise the same modifications and nucleotide analogs such as LNA or INA.

Preferably, the activity of the target RNA is expression and the target RNA is a mRNA.

Hence, oligonucleotides (candidate regulators) potentially capable of regulating the activity of a target RNA are first identified, where after the activity of these oligonucleotides are tested using a reporter system such as to verify whether the oligonucleotides do indeed have the desired activity, i.e. are capable of regulating the activity of the target RNA.

Preferably, the oligonucleotides provided in the third aspect of the invention are oligonucleotides of the invention.

The activity of the target RNA is preferably gene expression and the target RNA is preferably a mRNA. The target RNA may also be a viral genomic RNA and the activity e.g. replication.

The predetermined target sequence may be retrieved from a scientific publication or a database of validated microRNA targets.

### Reporter System

The reporter system for expression may be any system that enables a read-out indicative of the activity of the target RNA. It may be e.g. be cells harbouring a genetic construct, wherein the target RNA has been fused to another reporter gene.

In a preferred embodiment, the target sequence of the target RNA resides within the 3′-untranslated region of an mRNA. In such cases, the 3′UTR may be fused to a reporter gene without necessarily including the rest of the target mRNA.

The reporter gene may be e.g. the luciferase gene or GFP gene. Such reporter systems are well-known to the skilled man.

The reporter system could also be cells harbouring the endogenous target mRNA. In such an embodiment, the activity (expression) of the target mRNA may be determined by immunoblotting using antibodies targeting the polypeptide or protein encoded by the mRNA. 2D-gel analysis or protein chips may also be used to determine the activity of the target mRNA.

Microarrays, Northern blots and real time PCR (also known as quantitative PCR) may be used to determine any effects on mRNA levels. Also such reporter systems are well known to the skilled man.

### Using the Seed Sequence and Anti-Seed Sequence

In a preferred embodiment, the method of the third aspect further comprises providing the sequence of the microRNA regulating the target RNA and using the seed sequence of the microRNA to determine the anti-seed sequence of the target sequence.

The sequence of the microRNA regulating the target mRNA may e.g. be retrieved from a scientific paper or from a database. One such database collecting microRNA sequences is the so called miRBase (http://microrna.sanger.ac.uk/sequences/). In a preferred embodiment, the sequence of the microRNA is retrieved from a scientific paper describing regulation of the target mRNA by the microRNA. In another embodiment, the identity of the microRNA regulating the target mRNA is retrieved from a scientific publication, where after the sequence of the microRNAs is retrieved from a database. Such information is often the starting point for the method of the third aspect.

The seed sequence of the microRNA typically resides in the 5′end of the microRNA. Seed sequences are interesting, because it is believed that these are important predictors of the target mRNAs that are regulated by a particular microRNA. I.e. it is believed that they base-pair to complementary regions on target mRNAs. Such complementary regions of target mRNAs are herein also referred to as anti-seed regions or anti-seed sequences. Unfortunately, the seed sequences are often too short to allow prediction of target mRNAs, i.e. there are too many anti-seed sequences in the transcriptome of a cell. Thus, identification of target mRNAs regulated by a given microRNA still poses a significant challenge and so far hinge on experimental proof rather than theoretical prediction.

Nonetheless, progress is continually made with regards to determine which mRNAs are regulated by which microRNAs and it is an object of the present invention to use such knowledge to carry out the method of the third aspect and to design and provide oligonucleotides of the invention.

In a preferred embodiment, the target region of the target mRNA is comprised within the 3′UTR and comprise a sequence that is complementary to a sequence selected from the group consisting of: position 1-20, position 1-19, position 1-18, position 1-17, position 1-16, position 1-15, position 1-14, position 1-13, position 1-12, position 1-11, position 1-10, position 1-9, position 1-8, position 1-7, position 1-6, position 2-20, position 2-19, position 2-18, position 2-17, position 2-16, position 2-15, position 2-14, position 2-13, position 2-12, position 2-11, position 2-10, position 2-9, position 2-8, position 2-7, position 2-6, position 3-20, position 3-19, position 3-18, position 3-17, position 3-16, position 3-15, position 3-14, position 3-13, position 3-12, position 3-11, position 3-10 and position 3-9 of any SEQ ID NOs 1-723.

In a more preferred embodiment, the target region of the target mRNA is comprised within the 3′UTR and comprise a sequence that is complementary to a sequence selected from the group consisting of: position 1-10, position 1-9, position 1-8, position 1-7, position 1-6, position 2-10, position 2-9, position 2-8, position 2-7, position 2-6, position 3-10 and position 3-9 of any SEQ ID NOs:1-723.

In a most preferred embodiment, the target region of the target mRNA is comprised within the 3′UTR and comprise a sequence that is complementary to a sequence selected from the group consisting of: position 1-8, position 1-7, position 2-8 and position 2-7 of any SEQ ID NOs: 1-723.

Fourth Aspect—Identifying Target Regions, microRNA Regulators Thereof and Oligonucleotides of the Invention

In a fourth aspect, the invention provides a method comprising the steps
a. Providing a reporter system for activity of a target RNA
b. Providing a oligonucleotide that is complementary to a part of the target RNA
c. Determining the activity of the target RNA in the presence of the oligonucleotide of step b
d. Determining the activity of the target RNA in the absence of the oligonucleotide of step b
e. Comparing the activity levels in c and d and thereby verifying whether the oligonucleotide affect the activity of the RNA
f. Thereby identifying active oligonucleotides capable of regulating the activity of the target RNA and/or identifying microRNA target sequences of a RNA

Reporter systems have been described in the previous aspect.

One object of the oligonucleotides of the present invention is that they should prevent access of a microRNA to at least one of the target mRNAs of the particular microRNA. Thus, depending on the strength of oligonucleotide interaction with the target mRNA, the oligonucleotide will prevent the microRNA in base pairing with the target sequence. In other words, the microRNA is no longer able to guide the RNAi machinery to the target mRNA and exert its effects on the target mRNA.

In a preferred embodiment, the target region of the target RNA is the 3′UTR (3′untranslated region) of an mRNA.

In another preferred embodiment, the target region of the target mRNA is comprised within the 3′UTR.

In another embodiment, the method is a method of identifying a micro RNA target sequence of the RNA. I.e. microRNA targets of a given mRNA may e.g. be identified using the method of the fourth aspect.

In still another embodiment, the method is a method of identifying an oligonucleotide capable of regulating the activity of the RNA.

The method may further comprise providing a series of oligonucleotides that each are complementary to a part of the target RNA and where the series of oligonucleotides has an overall coverage of more than 50% for a particular target region of the target RNA and wherein each oligonucleotide is tested for activity (with respect to regulating the activity of the target RNA).

Preferably, the sequence of active oligonucleotides is used to define oligonucleotide sensitive regions of the target region. Moreover, the sequences of oligonucleotide sensitive regions are preferably used to design one or more oligonucleotide with optimized sequences, i.e. optimized activity.

In another embodiment, the sequences of the active oligonucleotides are truncated and tested for activity again such as to define minimal lengths of the oligonucleotides that will function as regulators of the mRNA.

As referred to herein, an oligonucleotide sensitive region is a region of the RNA, which when base paired to an oligonucleotide, affects the activity of the RNA. Typically, an oligonucleotide base paired to the oligonucleotide sensitive region will prevent a microRNA from regulating the activity of the RNA.

In a preferred embodiment, the sequences of oligonucleotide sensitive regions are used to identify candidate microRNAs that potentially regulate the target RNA. Thus, the method is a method of verifying which microRNAs regulate a given target RNA.

Identification of microRNAs that regulate a particular mRNA is of interest for various reasons. First, it will provide insight into how the RNAi machinery is recruited to particular mRNA targets and this information may be used to direct the RNAi machinery one or more therapeutic targets, e.g. mRNAs that encode proteins involved in disease. Second, a particular mRNA may be targeted for regulation by an antimir oligonucleotide that inhibits the activity of the microRNA regulating the activity of the mRNA. Determining which mRNAs are regulated by a particular microRNA or which microRNAs regulate a particular mRNA is currently one of, if not, the most important questions relating to RNAi, microRNAs and siRNAs.

It is an object of the present invention to provide such regulatory relationships between microRNAs and mRNAs.

Identification of candidate microRNAs preferably comprises the steps of:
a. Providing a sequence of an oligonucleotide sensitive region
b. Searching to sequence of the oligonucleotide sensitive region for complementarity to microRNAs to identify candidate microRNAs that potentially regulate the target RNA

When searching for complementarity, the seed sequence is particular important and the oligonucleotide sensitive region is preferably first searched for anti-seed sequences.

In a preferred embodiment, the activity of the identified candidate microRNAs that potentially regulate the target RNA is verified in a secondary test such as to identify microRNAs that do indeed regulate the activity of the target RNA

Preferably, the secondary test comprises the steps of:
a. Providing a reporter system for activity of the target RNA
b. Providing an antimir-oligonucleotide that comprises complementarity to the microRNA and is capable of inhibiting the activity of the candidate microRNA
c. Determining the activity of the target RNA in the presence of the antimir-oligonucleotide of step b
d. Determining the activity of the target RNA in the absence of the antimir-oligonucleotide of step b
e. Comparing the activity levels of step c and d and thereby verifying whether the identified candidate microRNA regulators are indeed active microRNA regulators of the target RNA

In a preferred embodiment, the secondary test further comprise a step of determining the expression of the target mRNA in the presence of the negative control, wherein said negative control is a oligonucleotide that do not comprise complementarity to the microRNAs.

Preferably, the method further comprises the steps of:
a. Determining the activity of the target RNA in the presence of an oligonucleotide directed to the target RNA
b. Determining the activity of the target RNA in the simultaneous presence of the oligonucleotide of step a in the presence of the antimir-oligonucleotide
c. Thereby verifying whether the oligonucleotide functions by blocking the activity of the micro RNA at the oligonucleotide sensitive region.

Thus, if the oligonucleotide has reduced or even no effect on the activity of the target RNA when the antimir is present, this indicates that the oligonucleotide functions by blocking the activity of the microRNA at the oligonucleotide sensitive region.

In a preferred embodiment, the coverage is selected from the group consisting of: more than 55%, more than 60%, more than 65%, more than 70%, more than 75%, more than 80%, more than 85%, more than 90%, more than 95%, more than 99% and 100%.

When referring to coverage, what is meant is the fraction of the target region that can be covered by the series of potential oligonucleotides. In other words, the fraction of target region that would be engaged in base pairing if the series of potential oligonucleotides where added to the target region under conditions of hybridisation.

In another preferred embodiment, the coverage is 100% and the oligonucleotides have an overlap in sequence.

In yet another preferred embodiment, a particular oligonucleotide has 50% overlap with the oligonucleotide to its 5′end and 50% overlap with the oligonucleotide to its 3′end. Thus, any give sequence of the target region will be covered by at least two oligonucleotides. Such a setup will be beneficial in defining oligonucleotide sensitive regions.

Preferably, the target RNA is a mRNA or a viral RNA. When the target RNA is a mRNA, the activity of the target mRNA is preferably gene expression.

If the target RNA is a target mRNA, the target region preferably is in the 3′UTR of the target mRNA.

In a preferred embodiment of the fourth aspect, the oligonucleotide is an oligonucleotide as described in the first aspect of the invention.

### Pharmaceutical Composition and Treatment

A fifth aspect of the present invention is a pharmaceutical composition comprising the oligonucleotide of the invention. As the skilled man will understand from the above description, the oligonucleotide may be used for therapy in the same manner as siRNAs, microRNAs and antisense oligonucleotides, because they can be used to specifically affect the expression of a particular gene.

A sixth aspect of the present invention is a method of treatment comprising administering an effective amount of the oligonucleotide of the invention or the pharmaceutical composition comprising the oligonucleotide of the invention to a person in need thereof.

A seventh aspect of the present invention is the oligonucleotide of the invention for use as medicine.

An eight aspect of the present invention is use of the oligonucleotide of the invention for the preparation of a medicament for treatment of cancer, viral infection, cardiovascular disease or immunogical disease.

The cancer may be glioblastoma, breast cancer, colorectal cancer and liver cancer.

The viral infection may be HIV infection, Hepatitis C infection, Hepatitis B infection, CMV infection and HSV infection.

The immunological disease may be psoriasis or eczema.

The cardiovascular disease may be treated by lowering high blood cholesterol.

A ninth aspect of the invention is use of the oligonucleotide of the invention for modulating the activity of a target RNA.

### Method of Transmission

A tenth aspect of the present invention is a method comprising transmission of information describing the oligonucleotide of the invention, oligonucleotide sensitive regions provided by the invention or information describing microRNA target regions of target RNAs provided by the invention. The information may describe either the oligonucleotide potentially capable of regulating the activity of a target mRNA or the oligonucleotide capable of regulating the activity of a target mRNA.

In a preferred embodiment of the tenth aspect, the transmission is electronic transmission.

### REFERENCES

Ameres S L, M. J. (2007). Molecular basis for target RNA recognition and cleavage by human RISC. Cell, July 13; 130(1):101-12.
Choi W Y, G. A. (2007). Target protectors reveal dampening and balancing of Nodal agonist and antagonist by miR-430. Science, October 12; 318(5848):271-4. Epub 2007 Aug. 30.
Gupta A, G. J. (2006). Anti-apoptotic function of a microRNA encoded by the HSV-1 latency-associated transcript. Nature, July 6; 442(7098):82-5.
Kawahara Y, Z. B. (2007). Redirection of silencing targets by adenosine-to-inosine editing of miRNAs. Science., February 23; 315(5815):1137-40.
Kertesz M, I. N. (2007). The role of site accessibility in microRNA target recognition. Nat Genet., October; 39(10):1278-84. Epub 2007 Sep. 23.
Long D, L. R. (2007). Potent effect of target structure on microRNA function. Nat Struct Mol Biol., April; 14(4):287-94. Epub 2007 April 1.
Poy M N, E. L. (2004). A pancreatic islet-specific microRNA regulates insulin secretion. Nature, November 11; 432 (7014), 226-30.
Xiao J, Y. B. (2007). Novel approaches for gene-specific interference via manipulating actions of microRNAs: examination on the pacemaker channel genes HCN2 and HCN4. J Cell Physiol., August; 212(2):285-92.

### EXAMPLES

### Example 1

### A Blockmir for Treatment of Diabetes

It has been demonstrated that mir-375 is a regulator of pancreatic island insulin secretion, and that Myotrophin (Mtpn) is a target of mir-375 regulation (Poy M N, 2004). Further, it has been shown that siRNA inhibition of Mtpn mimics the effects of miR-375 on glucose stimulated insulin secretion and exocytosis. Thus, it was concluded that by the authors that miR-375 is a regulator of insulin secretion and may thereby constitute a novel pharmacological target for the treatment of diabetes.

Here we provide blockmirs that can regulate Mtpn expression by inhibiting mir-375 regulation of Mtpn. activity on the 3′UTR of the Mtpn mRNA.

The relevant portion of the target mRNA is:

5′GUGUUUUAAGUUUUGUGUUGCAAGAACAAAUGGAAUAAACUUGAAU

The anti-seed sequence is shown in bold. This target region of the target RNA can be identified e.g. by searching the target RNA for anti-seed sequences. Or the target region can be found using suitable databases available on the internet (www.pictar.com, target-scan). Obviously, the information may also be available from experiments or from a scientific publication (as e.g. Poy et al.)

The sequence of mir-375 is:

5′UUUGUUCGUUCGGCUCGCGUGA

Pairing the seed sequence to the anti-seed sequence gives e.g. the following interactions.

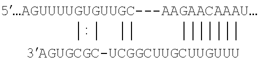

It is seen that overall complementarity is scarce.

### The Blockmir:

A blockmir capable of regulating Mtpn expression by inhibition of mir-375 regulation will have to be able to sequester the anti-seed sequence of the target region, i.e. hide the anti-seed sequence in base pairing.

Thus, blockmirs (lower strand in 3′-5′ direction) of Mtpn are exemplified here, base paired to the target sequence (upper strand in 5′-3′ direction):

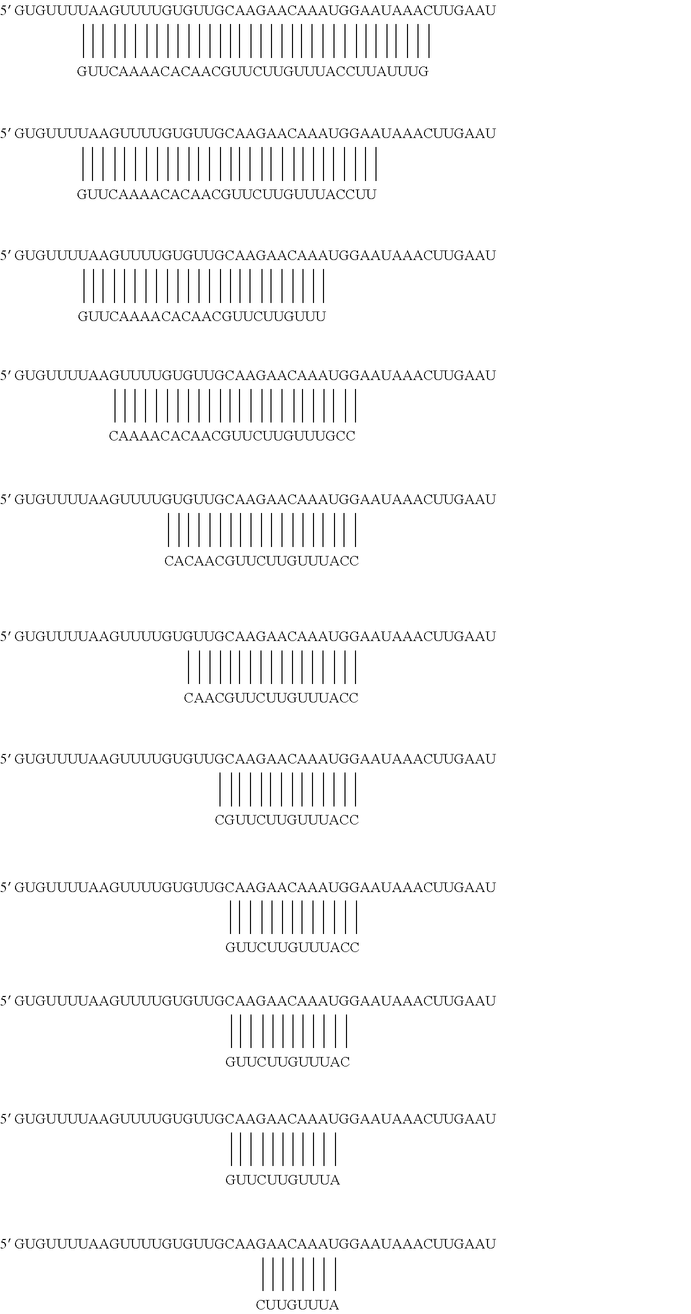

The blockmirs designed above may be synthesized using methods known in the art. As described in the specification, they may be synthesised as DNA, RNA, LNA, INA or with mixed monomers.

Obviously, U monomers may be exchanged with T monomers, while still allowing base pairing. Also G-C base pairs may be substituted with G-U wobble base pairs.

Methods for synthesizing various embodiments of the above designed blockmirs targeting the Mtpn mRNA are well known to the skilled man within the field of oligonucleotide synthesis. Particular preferred embodiments are described in the detailed description of the invention.

Conjugation of the blockmirs to e.g. cholesterol is also within the common knowledge of the skilled man.

### Example 2

### A Blockmir for Treatment of Herpes-Simplex Virus Infection

Recently it was demonstrated that Herpes simplex virus-1 encoded a microRNA that enables the virus to establish a latent infection (Gupta A, 2006). The microRNA that was termed mir-LAT was found to regulate TGF-beta and SMAD3, and thereby affect the ability of the cell to undergo apoptosis, the usual process by which an infected cell self-destructs in order to prevent production of viral progeny. Thus, it is of interest to be able to block the regulatory activity of mir-LAT on the expression of TGF-beta and SMAD3.

The sequence of the target region of the TGF-beta mRNA is:

5′AGGTCCCGCCCCGCCCCGCCCCGCCCCGGCAGGCCCGGCCCCACC

The sequence of mir-LAT is:

5′UGGCGGCCCGGCCCGGGGCC

Thus, the following complex may be formed:

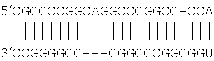

A series of blockmirs can be designed as was also done in the previous example. The lower strand is the blockmir shown in the 3′-5′ direction and the upper strand is the target region of TGF-beta mRNA:

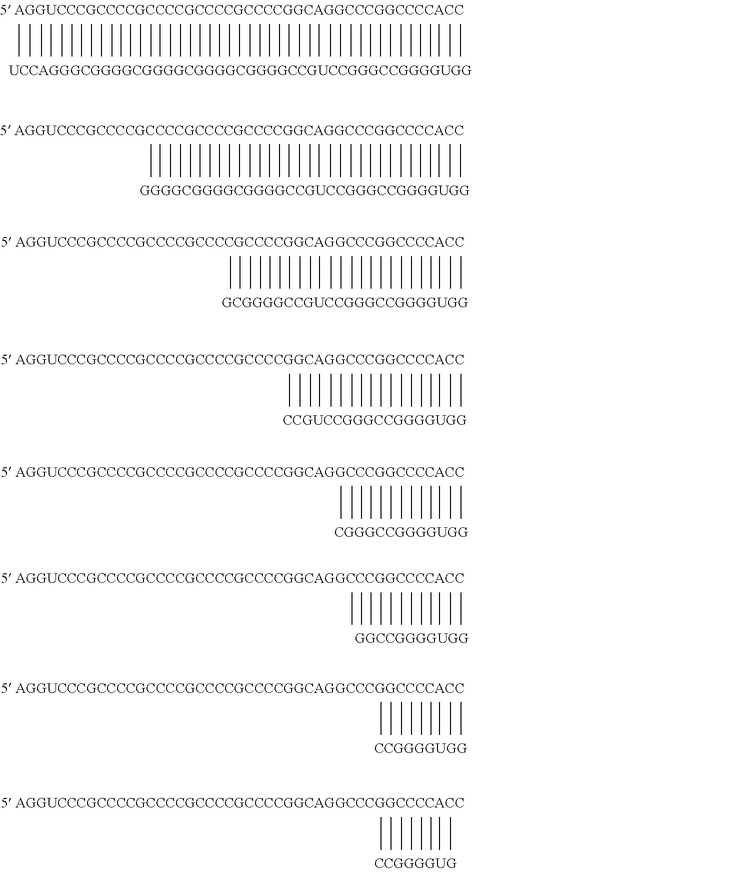

Synthesis of various embodiments of such sequences is well within the ability of the skilled man. Particular preferred embodiments are described in the detailed description of the invention.

### Example 3

### Identification of an Oligonucleotide that Regulate Expression of Mtpn

The following is a non-limiting example of how the method may be carried out. No wet experiments have actually been carried out.

The following sequence is a portion of the estimated target region of the Mtpn mRNA:

5′UUUGACGCAGUUGGGUUUCUCAUAAGUAUCCUAGUUCAUGUACAUCCG
AAUGCUAAAUAAUACUGUGUUUUAAGUUUUGUGUUGCAAGAACAAAGGAA
UAAACUUGAAUUGUGCUAC

A series of potential blockmirs for this region with a 50% overlap is designed. Potential blockmirs are shown in italic and a reference sequence of the Mtpn mRNA and a complementary strand are shown for comparison:

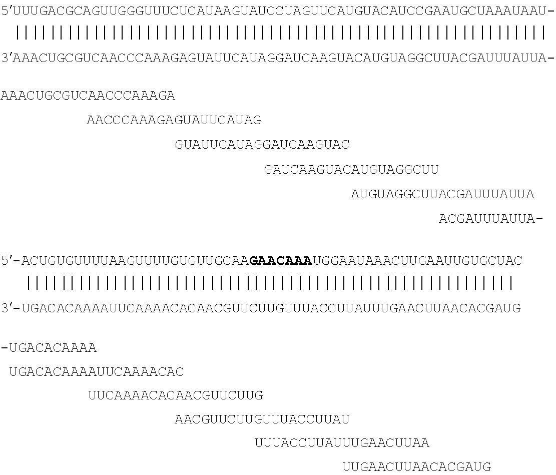

Thus, 11 blockmirs have been designed. These are then synthesised, e.g. as 2-O-modified oligonucleotides with phosphorothioate linkages. Methods for synthesizing various embodiments of the above designed blockmirs targeting the Mtpn mRNA are well known to the skilled man within the field of oligonucleotide synthesis. Particular preferred embodiments are described in the detailed description of the invention.

Conjugation of the blockmirs to e.g. cholesterol is also within the common knowledge of the skilled man

The target sequence is then fused to a reporter gene, which expression can be detected. In this example, the reporter gene is eGFP. The reporter gene is then transfected to Hela cells, where after expression of eGFP is monitored after transfection of each of the 11 designed blockmirs.

### Result:

Only blockmirs 7-9, counting from blockmirs complementary to the 5′end of the target region, affects the expression of eGFP. Blockmir 7 seven gives a slight increase in eGFP expression, whereas blockmir 8 and 9 has a more dramatic effect.

Thus, oligonucleotides that can affect the expression of the Mtpn mRNA have been identified.

The result indicates that the region covered by blockmirs 7-9 is a target for microRNA regulation. Furthermore, the result indicates that the region covered by blockmirs 8 and 9 is most important for microRNA regulation. The region covered by both oligonucleotide 8 and 9 may comprise the region that interact with the seed sequence of the microRNA or partly comprise the region that interact with the seed sequence of the microRNA.

The region covered by blockmirs 8 and 9 (AAGTTTTGTGTTGCAAGAACAAATGGAATA) is then searched for complementarity to microRNAs.

More specifically, the region is searched for complementarity to seed sequences of microRNA. This search identifies human mir-375.

Whether this microRNA does indeed regulate the activity of the Mtpn mRNA may be verified by inhibiting the activity of mir-375 with an antimir.

The sequence of mir-375 is:

5′UUUGUUCGUUCGGCUCGCGUGA

Thus, an inhibitory antimir with the complementary sequence is synthesized.

### Mir-375-Antimir:

5′TCACGCGAGCCGAACGAACAAA

Truncated versions of the antimir is also produced:

5′ACGCGAGCCGAACGAACAAA
5′GCGAGCCGAACGAACAAA
5′GAGCCGAACGAACAAA
5′GCCGAACGAACAAA
5′CGAACGAACAAA

The antimirs are synthesised e.g. as 2-modified oligonucleotides with phosphorothioate linkages. Then it is tested whether these antimirs can prevent mir-375 from regulating the Mtpn mRNA using the reporter system. The result shows that mir-375 does indeed regulate expression of the Mtpn mRNA.

1. An oligonucleotide comprising a antisense sequence that comprises a guide sequence corresponding to the seed sequence of a microRNA, with the proviso that the oligonucleotide is not a microRNA or does not comprise a sequence corresponding to the complete sequence of a microRNA.

2. The oligonucleotide of claim 1, wherein the microRNA is a human microRNA

3. The oligonucleotide of claim 1 comprising a sequence selected from the group consisting of sequences that are capable of base pairing to the complementary sequence of a sequence selected from the group consisting of position 1-20, position 1-19, position 1-18, position 1-17, position 1-16, position 1-15, position 1-14, position 1-13, position 1-12, position 1-11, position 1-10, position 1-9, position 1-8, position 1-7, position 1-6, position 2-20, position 2-19, position 2-18, position 2-17, position 2-16, position 2-15, position 2-14, position 2-13, position 2-12, position 2-11, position 2-10, position 2-9, position 2-8, position 2-7, position 2-6, position 3-20, position 3-19, position 3-18, position 3-17, position 3-16, position 3-15, position 3-14, position 3-13, position 3-12, position 3-11, position 3-10 and position 3-9 of any SEQ ID NOs:1-723

4. The oligonucleotide of claim 1, wherein the antisense sequence comprises an sequence selected from the group consisting of position 1-20, position 1-19, position 1-18, position 1-17, position 1-16, position 1-15, position 1-14, position 1-13, position 1-12, position 1-11, position 1-10, position 1-9, position 1-8, position 1-7, position 1-6, position 2-20, position 2-19, position 2-18, position 2-17, position 2-16, position 2-15, position 2-14, position 2-13, position 2-12, position 2-11, position 2-10, position 2-9, position 2-8, position 2-7, position 2-6, position 3-20, position 3-19, position 3-18, position 3-17, position 3-16, position 3-15, position 3-14, position 3-13, position 3-12, position 3-11, position 3-10 and position 3-9 of any SEQ ID NOs:1-723, wherein
a. A may be exchanged with only G, C, U, T or I
b. G may be exchanged with only A or I
c. C may be exchanged with only A, U or T
d. U may be exchanged with only C, A, T or I
and wherein 3 additional positions may be exchanged with any base.

5. The oligonucleotide of claim 3, wherein
a. A may be exchanged with only G, C, U, T or I
b. G may be exchanged with only A or I
c. C may be exchanged with only A or U
d. U may be exchanged with only C, A, T or I
and wherein 3 additional positions may be exchanged with any base.

6. The oligonucleotide of claim 3, wherein
a. A may be exchanged with only C, U, T or I
b. G may be exchanged with only I
c. C may be exchanged with only A, U or T
d. U may be exchanged with only C, A, T or I
and wherein 3 additional positions may be exchanged with any base.

7. The oligonucleotide of claim 3, wherein
a. A may be exchanged with only C, U, or I
b. G may be exchanged with only I
c. C may be exchanged with only A or U
d. U may be exchanged with only C, A, T or I
and wherein 3 additional positions may be exchanged with any base.

8. The oligonucleotide of claim 3, wherein
a. A may be exchanged with only G or I
b. G may be exchanged with only I or A
c. C may be exchanged with only A, U or T
d. U may be exchanged with only C or T
and wherein 3 additional positions may be exchanged with any base.

9. The oligonucleotide of claim 3, wherein
a. A may be exchanged with only G
b. G may be exchanged with only A or G
c. C may be exchanged with only T or U
d. U may be exchanged with only C or T
and wherein 3 additional positions may be exchanged with any base.

10. The oligonucleotide of claim 3, wherein U may be exchanged with only T
and wherein 3 additional positions may be exchanged with any base.

11. The oligonucleotide of claim 1, wherein 2 additional positions may be exchanged with any base.

12. The oligonucleotide of claim 1, wherein 1 additional position may be exchanged with any base.

13. The oligonucleotide of claim 1, wherein no additional positions may be exchanged with any base.

14. (canceled)

15. (canceled)

16. (canceled)

17. (canceled)

18. (canceled)

19. (canceled)

20. (canceled)

21. (canceled)

22. (canceled)

23. (canceled)

24. (canceled)

25. (canceled)

26. (canceled)

27. (canceled)

28. (canceled)

29. (canceled)

30. (canceled)

31. (canceled)

32. (canceled)

33. (canceled)

34. (canceled)

35. (canceled)

36. (canceled)

37. The oligonucleotide comprising a repeating pattern of one or more LNA units and one or more units that are substituted in the 2′-position.

38. (canceled)

39. The oligonucleotide of claim 1, wherein the oligonucleotide do not comprise any DNA units.

40. (canceled)

41. (canceled)

42. (canceled)

43. (canceled)

44. (canceled)

45. (canceled)

46. (canceled)

47. (canceled)

48. (canceled)

49. (canceled)

50. (canceled)

51. (canceled)

52. (canceled)

53. (canceled)

54. (canceled)

55. (canceled)

56. (canceled)

57. A method comprising the steps of:
a. Providing a target sequence of a target RNA regulated by a microRNA, said target sequence being the sequence of the target RNA involved in microRNA regulation.
b. Designing an oligonucleotide sequence that comprises a stretch of bases of at least 6 bases that is complementary to the target sequence
c. Synthesizing the oligonucleotide sequence of step b, thereby providing the oligonucleotide of step b, said oligonucleotide being a candidate regulator of the activity of a target RNA.

58. The method of claim 57 further comprising testing the steps of:
a. Providing a reporter system for activity of the target RNA
b. Determining the activity of the target RNA in the presence of the oligonucleotide of claim 57 step c
c. Determining the activity of the target RNA in the absence of the oligonucleotide of claim 57 step c
d. Comparing the activity levels in b and c and thereby verifying whether the oligonucleotide is indeed a capable of regulating the activity of the RNA and/or whether the potential target sequence of the RNA is indeed a target sequence.

59. (canceled)

60. The method of claim 57, wherein the target sequence of the target RNA comprises a sequence complementary to the seed sequence of a microRNAs.

61. (canceled)

62. (canceled)

63. (canceled)

64. (canceled)

65. (canceled)

66. (canceled)

67. (canceled)

68. (canceled)

69. (canceled)

70. (canceled)

71. (canceled)

72. A method comprising the steps of
a. Providing a reporter system for expression of a target mRNA
b. Providing a oligonucleotide that is complementary to a part of the target mRNA
c. Determining the expression of the target mRNA in the presence of the oligonucleotide of step b
d. Determining the expression of the target mRNA in the absence of the oligonucleotide of step b
e. Comparing the expression levels in c and d and thereby verifying whether the oligonucleotide affect the expression of the mRNA.

73. (canceled)

74. (canceled)

75. The method of claim 72, wherein a series of oligonucleotides are provided that each are complementary to a part of the target mRNA and where the series of oligonucleotides has an overall coverage of more than 50% for a particular target region of the target mRNA and wherein each oligonucleotide in the series are tested for activity.

76. (canceled)

77. (canceled)

78. (canceled)

79. (canceled)

80. (canceled)

81. (canceled)

82. (canceled)

83. (canceled)

84. (canceled)

85. (canceled)

86. (canceled)

87. (canceled)

88. (canceled)

89. (canceled)

90. (canceled)

91. (canceled)

92. (canceled)

93. (canceled)
